(19) United States
(12) Patent Application Publication
(10) Pub. No.: US 2014/0316248 A1
(43) Pub. Date: Oct. 23, 2014

(54) METHODS FOR MEASUREMENT AND ANALYSIS OF BRAIN ACTIVITY

(71) Applicant: R. Christopher deCharms, Montara, CA (US)

(72) Inventor: R. Christopher deCharms, Montara, CA (US)

(21) Appl. No.: 14/257,931

(22) Filed: Apr. 21, 2014

Related U.S. Application Data
(63) Continuation of application No. 13/889,304, filed on May 7, 2013, now abandoned.
Application No. 13/889,304 is a continuation of application No. 12/490,144, filed on Jun. 23, 2009, now abandoned.
Application No. 12/490,144 is a continuation of application No. 11/738,967, filed on Apr. 23, 2007, now abandoned.
Application No. 11/738,967 is a continuation of application No. 10/628,875, filed on Jul. 28, 2003, now abandoned.
(60) Provisional application No. 60/466,885, filed on May 1, 2003.
(60) Provisional application No. 60/399,055, filed on Jul. 26, 2002.

(51) Int. Cl. A61B 5/00; G01R 33/48; A61B 5/055

(52) U.S. Cl. CPC A61B 5/0048; A61B 5/055; G01R 33/4806
USPC 600/411

(57) ABSTRACT

A computer assisted method is provided for diagnosing the in condition of a subject associated with particular activation in one or more regions of interest, the method comprising: having the subject perform a behavior or have a perception adapted to selectively activate one or more regions of interest associated with the condition; measuring activity of the one or more regions of interest as the behavior is performed or the subject has the perception; diagnosing the condition associated with the one or more regions of interest based on the activity in response to the behavior or perception; performing an intervention; and repeating this process one or more times including repeating said behavior, said measuring of activity and said diagnosis at a later time; and observing changes between measurements that are associated with said intervention.

### CROSS-REFERENCE

This application is a continuation application of Ser. No. 13/889,304, entitled “Methods for Measurement And Analysis of Brain Activity,” filed on May 7, 2013, which is a continuation of Ser. No. 12/490,144, entitled “Methods for Measurement And Analysis of Brain Activity,” filed Jun. 23, 2009, which is a continuation of Ser. No. 11/738,967, entitled “Methods for Measurement And Analysis of Brain Activity,” filed Apr. 23, 2007, which is a continuation of Ser. No. 10/628,875, entitled “Methods for Measurement and Analysis of Brain Activity,” filed Jul. 28, 2003, which claims the benefit of U.S. Provisional Application No. 60/399,055, entitled “Methods For Measurement And Analysis Of Brain Activity,” filed Jul. 26, 2002, and which also claims benefit of U.S. Provisional Application No. 60/466,885, entitled “Methods for Physiological Diagnosis,” filed on May 1, 2003, all of which are incorporated herein by reference in their entirety.

### BACKGROUND OF THE INVENTION

The present invention relates to methods, software and systems for monitoring physiological activity, particularly in the human brain and nervous system and therapeutic and diagnostic applications relating thereto.

A large number of psychiatric (i.e. schizophrenia), neurological (i.e. Parkinson's disease), and neurodegenerative (i.e. Huntington's chorea) pathologies involve changes of mental states or conditions based upon changes in neurotransmitter and receptor balances. Detection of such changes may allow for diagnosis well ahead of manifestation of severe clinical symptoms, and knowledge of the nature and the extent of such changes is of paramount importance for the determination of therapy. For instance, in Parkinson's disease the chronic use of L-DOPA therapy leads to a progressive diminution in its efficacy. Thus, one would like to be able to monitor the progression of the disease more closely to effect possible changes in dosing. Similar problems present for many of the currently used dopaminergic ligands in schizophrenia. Determination of the effects of these therapies upon the brain is very difficult at the present time.

Two methodologies have been widely used for the determination of changes in neurotransmitter and receptor dynamics in vivo. These two techniques (Positron Emission Tomography and Single Photon Emission Computed Tomography, PET and SPECT) involve the use of radioactivity. Positron Emission Tomography is a very versatile technique which has been used successfully for the mapping of Cerebral Blood Flow (CBF), cerebral glucose metabolism (using .sup.18 F-fluorodeoxyglucose, FDG) or receptor activity (using radioactive pharmacological ligands), while SPECT is more limited to the detection of nonspecific processes. Unfortunately, both techniques suffer from severe limitations in spatial and temporal resolution, and cannot be proposed for repeated applications. Moreover, PET is characterized by limited availability and high costs, which are partly due to the short half-life of many of the radiopharmaceuticals which have to be administered.

A third alternative has recently been developed and is called pharmacological Magnetic Resonance Imaging (phMRI) and is based upon changes in Blood Oxygen Level Dependent (BOLD) contrast. The method rests on the spatially and temporally resolved visualization of the hemodynamic response evoked by neuronal activation following application of a specific pharmacological stimulus. Briefly: neuronal activation results in an increased local metabolic activity, increased oxygen consumption and increased local concentration of paramagnetic deoxyhemoglobin. Since the latter is compartmentalized in the vasculature, its higher magnetic susceptibility leads to a decreased Signal Intensity (SI) of brain tissue in T.sub.2*-weighted MR images. This effect is however quickly overcompensated by increased relative Cerebral Blood Flow (rCBF), with consequent inflow of fresh blood with lower content in deoxyhemoglobin, leading finally to increased SI on T.sub.2*-weighted images in the area of neuronal activation.

While phMRI offers the needed high spatial and temporal resolution as well as the non-invasiveness of MRI, it suffers from the lack of sensitivity of the BOLD effect, which amounts to an increase in SI of only 2-3% at clinical field strengths. This is by far not enough for the establishment of a robust clinical procedure. This problem has been dealt with, with better results, for the analogous technique called functional MRI (fMRI), which differs from phMRI by the nature of the stimulus which is sensorial or motor rather than pharmacological. In fMRI, the low intensity of the BOLD effect is compensated by repeated acquisition of alternating data blocks at rest and under stimulation and using statistical approaches like Multivariate Analysis of Covariance (ManCova) to generate Statistical Parameter Maps (SPM) which represent the statistical significance—on a pixel-by-pixel basis—of any differences in SI between scans taken at rest and during stimulation. However, this solution is not applicable to phMRI due to the long duration (typically 1 hour) of the response to pharmacological stimulation, as opposed to the short duration (seconds) to sensorial or motor stimulation.

A variety of different brain scanning methodologies have been developed that may be used to identify changes of mental states or conditions including Positron Emission Tomography (PET) and Single Photon Emission Computed Tomography (SPECT), electroencephalogram (EEG) based imaging, magnetoencephalogram (MEG) based imaging, and functional magnetic resonance imaging (fMRI).

For example, magnetic resonance imaging (MRI) has been used successfully to study blood flow in vivo. U.S. Pat. Nos. 4,983,917, 4,993,414, 5,195,524, 5,243,283, 5,281,916, and 5,227,725 provide examples of the techniques that have been employed. These patents are generally related to measuring blood flow with or without the use of a contrast bolus, some of these techniques referred to in the art as MRI angiography. Many such techniques are directed to measuring the signal from moving moieties (e.g., the signal from arterial blood water) in the vascular compartment, not from stationary tissue. Thus, images are based directly on water flowing in the arteries, for example. U.S. Pat. No. 5,184,074, describes a method for the presentation of MRI images to the physician during a scan, or to the subject undergoing MRI scanning.

In the brain, several researchers have studied perfusion by dynamic MR imaging using an intravenous bolus administration of a contrast agent in both humans and animal models (See, A. Villringer et al, Magn. Reson, Med., Vol. 6 (1988), pp 164-174; B. R. Rosen et al, Magn. Reson. Med., Vol. 14 (1999), pp. 249-265; J. W. Belliveau et al, Science, Vol. 254 (1990), page 716). These methods are based on the susceptibility induced signal losses upon the passage of the contrast agent through the microvasculature. Although these methods do not measure perfusion (or cerebral blood flow, CBF) in classical units, they allow for evaluation of the related variable rCBV (relative cerebral blood volume). For example, in U.S. Pat. No. 5,190,744 to Rocklage, quantitative detection of blood flow abnormalities is based on the rate, degree, duration, and magnitude of signal intensity loss which takes place for a region following MR contrast agent administration as measured in a rapid sequence of magnetic resonance images. Other methods of monitoring brain activity are disclosed in U.S. application Ser. Nos. 10/066,004 and 10/062,627, both entitled “Method For Physiological Monitoring, Training, Exercise And Regulation,” and both filed Jan. 30, 2002, incorporated herein by reference for all purposes.

With the advent of these brain scanning methodologies, the absolute level of blood flow in various brain areas has been effectively correlated with various brain disorders such as Attention Deficit Disorder (ADD), Schizophrenia, Parkinson's Disease, Dementia, Alzheimers Disease, Endogenous Depression, Oppositional Defiant Disorder, Bipolar Disorder, memory loss, brain trauma, Epilepsy and others.

### SUMMARY OF THE INVENTION

The present invention is directed to various methods relating to the measurement in real time of fluctuations of physiological activity due to instructions or other stimulation, comparison of these measurements between people or groups, and use of this process in diagnosis.

### BRIEF DESCRIPTION OF THE DRAWINGS

FIG. 1 is an overview diagram of methods, components and processes of this invention.

FIG. 2 is a diagram of methods and apparatus for displaying information to a subject in a measurement apparatus.

FIG. 3 shows a table of brain regions that may be used as regions of interest.

FIG. 4 shows example display screens that may be used by the apparatus.

FIG. 5 shows further example display screens that may be used by the apparatus.

### DETAILED DESCRIPTION OF THE INVENTION

The term “activity,” as used herein, refers to physiological activity associated with one or more voxels of the brain whose physiological activity may be monitored. Examples of types of physiological activity include, but are not limited to, neuronal activity, blood flow, blood oxygenation, electrical activity, chemical activity, tissue perfusion, the level of a nutrient or trophic factor, the production or distribution of a trophic factor, the production, release, or reuptake of a neurotransmitter or neuromodulator, the growth of tissue such as neurons or parts of neurons, neural plasticity, and other physiological processes. Other examples are provided herein.

The term “activation,” as used herein, refers to a change in activity in one or more voxels of the brain whose physiological activity may be monitored. This change may include an increase or decrease. It is noted that this change may also include a change where some voxels increase in activation at the same time that other voxels decrease in activation.

The term “activity metric,” as used herein, refers to any computed measure of activity of one or more regions of interest of the brain.

The term “behavior,” as used herein, refers to a physical or mental task or exercise engaged in by a subject, which may be in order to activate one or more regions of interest of the brain. Examples of different types of behaviors include, but are not limited to sensory perception, detection or discrimination, motor activities, cognitive processes such as mental imagery or mental manipulation of an imagined object, reading, emotional tasks such as attempting to create a particular affect or mood, verbal tasks such as listening to, comprehending, or producing speech. A behavior may also include a state or set of acts undertaken by a subject caused by or in response to an intervention. As an example, a subject may engage, or cease engaging in, hallucinatory behaviors that are brought about by a pharmacological agent or prevented by a pharmacological agent. Other examples of behaviors are provided herein.

The term “BOLD,” as used herein refers to Blood Oxygen Level Dependent signal. This signal is typically measured using a functional magnetic resonance imaging device.

The term “condition,” as used herein, refers to any physiological, psychological or health condition that may be treated according to the present invention by changing a level of activity in one or more regions of interest associated with that condition. Numerous examples of conditions that may be treated according to the present invention are provided herein. It is noted that a condition may additionally refer to a normal state of a subject that one may desire to alter, such as the condition of a subject's mood, or something that the subject has learned, or a disease condition.

The term “diagnosis,” as used herein, refers to the determination of a condition of a subject, such as determining whether the subject has a particularly disease condition, susceptibility, or other trait.

The term “device operator,” as used herein, refers to an individual who controls the functioning of apparatus or software associated with this invention. It is to be noted that the device operator may be a person other than the subject, may be the subject, or may be a remotely located party using appropriate communication technology such as an internet connection.

The term “event related,” as used herein, refers to an event that is related to a physiological activity which is caused by a known event, or takes place immediately preceding or subsequent to that event. In a typical example, a stimulus or behavior event is repeated many times, and the average event related activity is the average activity level at a set of defined times relative to the onset time of the event. This may be computed using a PETH.

The term “existing MRI/fMRI/PET data processing packages,” as used herein refers to the following packages, their documentation, websites, and cited literature references contained in their documentation and websites: SPM99 (and the SPM99 manual written by Dick Veltman and Chloe Hutton, May 2001), Brain Voyager from Brian Innovation, AIR by Roger Woods, MRICro by Chris Rorden, AFNI by R W Cox, and other packages that may be developed to perform related functions.

The term “information,” as used herein, refers to anything communicated to the subject, whether by sight, sound, smell, contact with the subject, etc., relating to the performance of the various methods of the present invention. Examples of various types of information that may be communicated to the subject include, but are not limited to, instructions, physiological measurement related information, subject performance related information, and stimulus information that causes the subject to have a perception. Examples of ways of communicating information include, but are not limited to displaying information to the subject, playing audio for the subject, providing an agent for the subject to smell, applying a physical force to the subject (e.g., a pressure or vibration or proprioceptive stimulus), and causing a physical sensation for the subject (e.g., cold, hot, pain, electrical charge, etc.). Specific examples of information include, but are not limited to images of the subject's brain activity pattern, charts of the timecourse of physiological activity in a region of interest, or an activity metric from a region of interest, instructions to perform a task or how to perform a task, movies, or stereoscopic virtual reality stimuli viewed through stereo viewers and designed to simulate certain circumstances or experiences. Further examples include games played by the subject, such as computer games.

The term “instructions,” as used herein, refers to any instruction to perform a physical or mental action that is communicated to a subject or an operator assisting a subject. Examples of instructions include, but are not limited to instructions to a subject to perform a behavior; instructions to a subject to rest; instructions to a subject to move; instructions to a subject to make a computer input; instructions to a subject to activate a brain region, such as to a designated level. Further examples of instructions are provided herein.

The term “intervention,” as used herein refers to any manipulation of a subject. This includes pharmacological interventions, such as the administration of a pharmacological agent, stimulatory manipulations, such as the application of current to the nervous system using a stimulation device (e.g. deep brain stimulation), non-invasive stimulatory manipulations, such as the application of a stimulus to the nervous system using trans-cutaneous magnetic stimulation or another non-invasive stimulation modality, and behavioral manipulations such as rehabilitative therapy or behavioral therapy.

The term “localized region,” as used herein refers to any region of the brain with a defined spatial extent. In one variation, a localized region measured by this invention may be internal relative to a surface of the brain.

The term “measurement information,” as used herein, refers to any information that communicates a measurement to a subject. Examples of types of measurements include, but are not limited to anatomical measurements, physiological measurements, activity measurements, activity metrics computed from activity measurements, and activation images.

The term “measurement of activity,” as used herein, refers to the detection of activity in one or more voxels of the brain. Once measured, activity metrics may be computed from these measurements. Activity measurements may be performed by any measurement technology that is capable of measuring activity in one or more voxels of the brain, or by combinations of such technologies with other forms of measurement. Various suitable measurement technologies are described herein.

The term “neuromoanatomical texts,” as used herein refers to any of a variety of texts describing the structures of the brain, including but not limited to Fundamental Neuroanatomy by Nauta and Feirtag, and in the Co-Planar Steriotaxic Atlas of the Human Brain by Jean Talairach and Pierre Tournoux, Magnetic Resonance Imaging of the Brain and Spine (2 Volume Set) by Scott W., Md. Atlas.

The term “neuromodulator or neuromodulatory substance,” as used herein, refers to compounds which can alter activity or responsiveness in one or more localized regions of the brain. Examples of neuromodulators include, but are not limited to: opioids, neuropeptides, acetylcholine, dopamine, norepinephrine, serotonin and other biologic amines, and others. Many pharmacological agents such as morphine, caffeine and prozac are exogenous mimics of these neuromodulatory substances.

The term “PETH,” as used herein, refers to a pen-event time histogram. This is a measure of the average value of an activity pattern metric based upon multiple trials, for each of a set of fixed time intervals after a conditioning event such as a stimulus or the onset of a behavior.

The term “pharmacological treatment,” as used herein, refers to the administration of any type of drug, remedy, or medication.

The term “rest,” as used herein, refers to a period during which a subject is not engaged in a particular overt behavior. This may mean that the subject has received no instructions, that they have just received the instruction to remain still during measurement, that they have received the instruction to perform a ‘background task’ that leads to little brain activation in a measured region (such as pressing left and right buttons when corresponding arrows are displayed), or that they are drowsy or in a state of sleep.

The term “region of interest or ROI or volume of interest,” as used herein, refers to a particular one or more voxels of the brain of a subject. An ROI may occasionally be referred to as an area or volume of interest since the region of interest may be two dimensional (area) or three dimensional (volume). Frequently, it is an object of the methods of the present invention to monitor, control and/or alter brain activity in the region of interest. For example, the one or regions of interest of the brain associated with a given condition may be identified as the region of interest for that condition. In one variation, the regions of interest targeted by this invention are internal relative to a surface of the brain.

The term “scan volume,” as used herein, refers to a three dimensional volume within which brain activity is measured. This volume may be divided into an array of voxels. For example, in the case of fMRI, a scanning volume may correspond to a 3-D cube (e.g., 22×22×12 cm) that comprises the volume of the head of a subject. This volume may be divided into a 64×64×17 array of subvolumes (voxels).

The term “single point,” as used herein, refers to an individual geometric locus or small area of volume, such as a single small geometric volume from which a physiological measurement will be made, with the volume being 0.1, 0.5, 1, 2, 3, 4, 5, 10, 15, 20, 30, 50, 100 mm in diameter. A device making a measurement from a single point is contrasted with a device making scanned measurements from an entire volume comprised of many single points.

The term “spatial array,” as used herein, refers to a contiguous or non-contiguous set of location points, areas or volumes in space. The spatial array may be two dimensional in which case elements of the array are areas or three dimensional in which case elements of the array are volumes.

The term “spatial pattern, or spatial activity pattern, or vectorized spatial pattern,” as used herein, refers to the measured activities of the set of voxels forming a two dimensional or three dimensional spatial array such as a scan volume or portion of a scan volume. A vector comprising a rational or real value for each voxel in a three dimensional spatial array is one example of a spatial pattern. Since activity associated with each voxel is represented, a spatial pattern contains much more information than a single activity metric for the entire localized region. It is noted that a spatial pattern may be defined either in geometric space as physically measured, or may be defined in a transformed space or standard coordinate space intended to allow the geometric points in the brain of one subject to be aligned with anatomically or physiologically corresponding points in another subject or group of subjects.

The term “SSFP,” as used herein, refers to measurements of MR steady state free precession, which may be used as an indicator for anatomy or physiological function. Example implementation is described in Miller, K. L., Hargreaves, B. A., Lee, J., Ress, D., deCharms, R. C., and Pauly, J. M., Functional Brain Imaging Using a Blood Oxygenation Sensitive Steady-state. Magn Res Med in press.

The term “subject,” as used herein, refers to a person or animal whose brain activity is to be measured in conjunction with performing the methods of the present invention. It is noted that the subject is the person or animal who has the condition being treated or tested by the methods of the present invention.

The term “substantially real time,” as used herein, refers to a short period of time between process steps. Preferably, something occurs in substantially real time if it occurs within a time period of less than 10 seconds, more preferably less than 5, 4, 2, 1, 0.5, 0.2, 0.1, 0.01 seconds or less. In one particular embodiment, computing an activity metric is performed in substantially real time relative to when the brain activity measurement used to compute the activity metric was taken. In another particular embodiment, communicating information based on measured activity is performed in substantially real time relative to when the brain activity measurement was taken. Because activity metrics and information communication may be performed in substantially real time relative to when brain activity measurements are taken, it is thus possible for these actions to be taken while the subject is still in position to have his or her brain activity measured.

The term “task,” as used herein, refers to a perceptual, cognitive, behavioral, emotional, or other activity undertaken by a subject, typically repetitively as part of a trial.

The term “vectorized brain states,” as used herein, refers to a measured state of the brain where the activity in each voxel of the brain may be separately measured, as in a spatial activity pattern.

The term “voxel,” as used herein, refers to a point or three dimensional volume from which one or more measurements are made. A voxel may be a single measurement point, or may be part of a larger three dimensional grid array that covers a volume.

### I. In General

The present invention is directed to various methods relating to the use of behaviors performed by a subject and/or perceptions made by a subject or other manipulations or agents that alter the activity of one or more brain regions of interest. It should be recognized that this alteration in activation may be a decrease or increase in activity at the different regions of interest.

One particular aspect of the invention relates to the communication to a subject of information in combination with measuring the activation of the one or more regions of interest of the subject where the what, when, and/or how the information is communicated is determined, at least partially, based on the measured activity. Preferably, activity measurements are made continuously so that what, when, and/or how information is communicated to a subject in view of the activity measurements can be continuously determined Examples of types of information that may be controlled in this manner include, but are not limited to instructions, stimuli, physiological measurement related information, and subject performance related information.

Another particular aspect of the invention relates to the use of behaviors performed by a subject and/or perceptions made by a subject that alter the activity of one or more regions of interest in combination with measuring the activation of the one or more regions of interest. The measurement may be performed in substantially real time relative to the behavior or perception. Activation metrics may be calculated based on the measured activity and used to monitor changes in activation. These activation metrics may be used in diagnosis of a condition of the subject. These activation metrics may also be used in the testing of the effects of an exogenous agent or treatment.

The present invention also relates to systems that may be used in combination with performing the various methods according to the present invention. These systems may include a brain activity measurement apparatus, such as a magnetic resonance imaging scanner, one or more processors and software according to the present invention. These systems may also include means to present information to a device operator during testing, or upon completion of testing, or at a later time. These systems may also include software for automated diagnosis of the subject, or testing of brain activation metrics. These systems may also include mechanisms for communicating information such as instructions, stimulus information, physiological measurement related information, and/or subject performance related information to the subject or an operator. Such communication mechanisms may include a display, preferably a display adapted to be viewable by the subject while brain activity measurements are being taken. The communication mechanisms may also include mechanisms for delivering audio, tactile, temperature, or proprioceptive information to the subject. In some instances, the systems further include a mechanism by which the subject may input information to the system, preferably while brain activity measurements are being taken.

The present invention also relates to software that is designed to perform one or more operations employed in combination with the methods of the present invention. The various operations that are or may be performed by software will be understood by one of ordinary skill, in view of the teaching provided herein.

In one embodiment, a method is provided for testing activation of one or more regions of interest of a subject, the method comprising: evaluating a set of behaviors that a subject separately performs regarding how well each of the behaviors in the set activate the one or more regions of interest; and selecting a subset of the behaviors from the set found to be effective in activating the one or more regions of interest. In one variation, evaluating the set of behaviors comprises calculating and comparing activation metrics computed for each behavior based on measured activities for the different behaviors. In one variation, the behaviors evaluated are overt behaviors involving a physical motion of the body of the subject. In another variation, the behaviors are covert behaviors only cognitive processes which do not lead to a physical motion of the body of the subject.

In another embodiment, computer executable logic is provided for selecting how to achieve activation and testing of one or more regions of interest of a subject, the software comprising: logic for calculating activation metrics for activity measured for one or more regions of interest during for a plurality of different behaviors; and logic for comparing the calculated activation metrics for the plurality of behaviors and testing the effects of an intervention from the plurality based on the comparison of activation metrics.

In another embodiment, computer executable software is provided for guiding brain activity training comprising: logic which takes data corresponding to activity measurements of one or more internal voxels of a brain and determines one or more members of the group consisting of: a) what next stimulus to communicate to the subject, b) what next behavior to instruct the subject to perform, c) when a subject is to be exposed to a next stimulus, d) when the subject is to perform a next behavior, e) one or more activity metrics computed from the measured activity, f) a spatial pattern computed from the measured activity, g) a location of a region of interest computed from the measured activity, h) performance targets that a subject is to achieve computed from the measured activity, i) a performance measure of a subject's success computed from the measured activity, j) a subject's position relative to an activity measurement instrument; and logic for communicating information based on the determinations to the subject in substantially real time relative to when the activity is measured.

In another embodiment, a method is provided for directing and testing behavior, the method comprising: employing computer executable logic to select in substantially real time a next behavior for a subject to perform during training based, at least in part, on activity measurements made at or before the time the selection is made, and employing said activity measurements in diagnosing a condition of the subject.

In another embodiment, a method is provided for selecting a behavior for causing activation of one or more regions of interest of a subject, the method comprising: employing computer executable logic to select a next behavior for a subject to perform during training based, at least in part, on one or more behaviors previously used during training. In a variation, the selection is based on a combination of the one or more behaviors previously used during training and the activity measurements associated with the behaviors.

In another embodiment, a method is provided, the method comprising: evaluating a set of behaviors that a subject may undertake regarding how well each of the behaviors activate the one or more regions of interest; selecting a subset of the behaviors from the set found to be effective causing activation of the one or more regions of interest; applying stimuli leading to these behaviors; measuring resulting brain activation at one or more time points; and using these resultant brain activation measures in diagnosis of the subject. In one variation, evaluating the resultant brain activation is used in testing the efficacy of an intervention that is performed between two or more measurement time points. These two or more measurement time points may take place on different days, or they may take place while the subject remains within the measurement apparatus.

In another embodiment, computer executable logic is provided for testing activation of one or more regions of interest of a subject, the software comprising: logic for calculating activation metrics for activity measured for one or more regions of interest; and logic for comparing a set of calculated activation metrics with expected values corresponding to one or more diagnostic level or category.

In another embodiment, computer assisted method is provided comprising: measuring activity of one or more internal voxels of a brain; employing computer executable logic that takes the measured brain activity and determines one or more members of the group consisting of: a) what next stimulus to communicate to the subject, b) what next behavior to instruct the subject to perform, c) when a subject is to be exposed to a next stimulus, d) when the subject is to perform a next behavior, e) one or more activity metrics computed from the measured activity, 1) a spatial pattern computed from the measured activity, g) a location of a region of interest computed from the measured activity, h) performance targets that a subject is to achieve computed from the measured activity, i) a performance measure of a subject's success computed from the measured activity, j) a subject's position relative to an activity measurement instrument; k) the effect on the measured activity of an intervention; l) an estimate of a condition of the subject computed from the measured activity; and communicating information based on the determinations to the subject or device operator.

In another embodiment, computer executable logic is provided for selecting how to achieve activation and testing of one or more regions of interest of a subject, the software comprising: logic for calculating activation metrics for activity measured for one or more regions of interest during for a plurality of different behaviors; and logic for comparing the calculated activation metrics for the plurality of behaviors and selecting behaviors from the plurality based on the comparison of activation metrics.

In another embodiment, a method is provided for selecting how to test activation of one or more regions of interest of a subject, the method comprising: evaluating a set of stimuli that a subject is separately exposed to regarding how well each of the different stimuli cause the subject to have a perception that activates the one or more regions of interest; and selecting a subset of the stimuli from the set found to be effective in causing activation of the one or more regions of interest. In one variation, evaluating the set of stimuli comprises calculating and comparing activation metrics computed for each stimuli based on measured activities for the different stimuli. In another variation, the activation in the regions of interest is used as an indicator in diagnosis of a condition of the subject. In another variation, the activation in the regions of interest is used as an indicator in testing the efficacy of an intervention.

In another embodiment, computer executable logic is provided for selecting how to achieve activation and testing of one or more regions of interest of a subject, the software comprising: logic for calculating activation metrics for activity measured for one or more regions of interest during for a plurality of different behaviors; and logic for comparing the calculated activation metrics for the plurality of behaviors and diagnosing a condition of the subject from the plurality based on the comparison of activation metrics.

In another embodiment, a method is provided for selecting a behavior for testing activation of one or more regions of interest of a subject, the method comprising: employing computer executable logic to select in substantially real time a next behavior for a subject to perform during training based, at least in part, on activity measurements made at or before the time the selection is made. In one variation, the activity measurements are used to diagnose a condition of the subject. In another variation, the activity measurements are used to diagnose a condition of the subject and two or more time points. In another variation, the activity measurements are used to diagnose a condition of the subject and two or more time points with an intervention to be tested taking place during the intervening time.

In another embodiment, a method is provided for selecting a behavior for testing activation of one or more regions of interest of a subject, the method comprising: employing computer executable logic to selecting a next behavior for a subject to perform during training based, at least in part, on measured activities of one or more regions of interest in response to the performance of one or more earlier behaviors. In a variation, the selection is based on a combination of the measured activity and the identity of the one or more earlier behaviors. It is noted that the computer executable logic may optionally compute activity metrics from the measured activity for the one or more earlier behaviors and base the selection on the activity metrics. Optionally, the computed activity metrics are based on a comparison with a rest state. Optionally, the computed activity metrics are used in the diagnosis of a condition of a subject. Optionally, the computed activity metrics are used in the testing of the efficacy of an intervention.

In another embodiment, a method is provided for selecting a stimulus for causing and testing activation of one or more regions of interest of a subject, the method comprising: employing computer executable logic to select in substantially real time a next stimulus to communicate to a subject during training based, at least in part, on activity measurements made at the time the selection is made.

In another embodiment, a method is provided for selecting a stimulus for causing and testing activation of one or more regions of interest of a subject, the method comprising: employing computer executable logic to select a next stimulus to communicate to a subject during training based, at least in part, on one or more stimuli previously communicated during training. In a variation, the selection is based on a combination of the one or more stimuli previously communicated and the activity measurements associated with the stimuli.

In another embodiment, a method is provided for selecting a stimulus for causing and testing activation of one or more regions of interest of a subject, the method comprising: employing computer executable logic to select a next stimulus to communicate to a subject during training based, at least in part, on measured activities of one or more regions of interest in response to the communication of one or more earlier stimuli. In a variation, the selection is based on a combination of the measured activity and the identity of the one or more earlier stimuli. It is also noted that the computer executable logic may optionally compute activity metrics from the measured activity for the one or more earlier stimuli and base the selection on the activity metrics. Optionally, the computed activity metrics are based on a comparison with a rest state.

In regard to the above embodiments, it is noted that the next behavior or stimulus that is selected may be the same or different than the one or more earlier behaviors or stimuli.

In another embodiment, a computer assisted method is provided for guiding and testing brain activity training comprising: measuring activity of one or more regions of interest of a subject; employing computer executable logic to select a behavior or stimulus for activating the one or more regions of interest based, at least in part, on the measured brain activity; and employing computer executable logic to communicate the selected behavior or stimulus to the subject or device operator. In one variation, the method further comprises communicating information to the subject regarding the measured brain activity.

In another embodiment, software is provided for guiding brain activity training, the software comprising: computer executable logic for selecting a behavior or stimulus for activating one or more regions of interest of a subject based, at least in part, on a measured brain activity; and logic for communicating the selected behavior or stimulus to the subject. In one variation, the software further comprises logic that communicates information to the subject regarding the measured brain activity.

In another embodiment, a computer assisted method is provided for guiding brain activity training comprising: having a subject perform a first behavior or be exposed to a first stimulus; measuring activity of one or more regions of interest of the subject in response to the first behavior or first stimulus; and employing computer executable logic to select a second behavior or a second stimulus for activating the one or more regions of interest based, at least in part, on the measured brain activity; and having the subject perform the second behavior or be exposed to the second stimulus. Optionally, the method further comprises employing computer executable logic to communicate to the subject the selected second behavior or second stimulus.

In another embodiment, a computer assisted method is provided for guiding brain activity training comprising: instructing a subject to perform a first behavior or communicating a first stimulus to the subject; measuring activity of one or more regions of interest of the subject in response to the first behavior or first stimulus; and employing computer executable logic to select a second behavior or a second stimulus for activating the one or more regions of interest based, at least in part, on the measured brain activity; and instructing the subject to perform the second behavior or communicating the second stimulus to the subject.

Computer executable software is provided for guiding brain activity testing, the software comprising: logic for communicating instructions to a subject to perform a first behavior and/or a first stimulus to the subject; logic for taking activity measurements of one or more regions of interest of the subject in response to the first behavior or first stimulus and selecting a second behavior or a second stimulus for activating the one or more regions of interest based, at least in part, on the measured brain activity; and logic for communicating instructions to the subject to perform the second behavior and/or the second stimulus to the subject.

In another embodiment, computer executable software is provided for guiding and testing brain activity training, the software comprising: logic for measuring activity of one or more regions of interest of the subject in response to a first behavior or first stimulus; logic for selecting a second behavior or a second stimulus for activating the one or more regions of interest based, at least in part, on a measured brain activity; logic for communicating to the subject the selected second behavior or second stimulus.

In another embodiment, a method is provided for directing training and testing of one or more regions of interest of a subject, the method comprising: continuously measuring activity in the one or more regions of interest of the subject; and employing computer executable logic to determine when to communicate information to the subject based, at least in part, on the measured activities. It is noted that the computer executable logic may optionally compute activity metrics from the measured activity and base the selection on the activity metrics. The computer executable logic may determine when to communicate information based on when the computed activity metric satisfies a predetermined condition, such as a target activity metric. It is noted that the information may be instructions, stimuli, physiological measurement related information, and/or subject performance related information. In one variation, the instructions are instructions to perform a behavior.

In another embodiment, a method is provided for directing testing of one or more regions of interest of a subject, the method comprising: measuring activity in the one or more regions of interest of the subject; determining one or more activity metrics for the measured activity; determining when the one or more activity metrics satisfy a predetermined condition; and communicating information to the subject; wherein these steps are repeatedly performed in substantially real time.

In another embodiment, software is provided for directing testing of one or more regions of interest of a subject, the software comprising: logic for taking measurements of activity of the one or more regions of interest of the subject and determining one or more activity metrics for the measured activity; logic for determining when the one or more activity metrics satisfy a predetermined condition; and logic for causing information to be communicated to the subject; wherein the software is able to determine the activity metrics from the activity measurements and cause information to be communicated in substantially real time.

In another embodiment, a method is provided for directing testing, the method comprising: measuring activities of one or more regions of interest; determining when the measured activities have reached a desired state; and communicating information to a subject regarding when to perform a next behavior when the measured activities have reached the desired state.

In another embodiment, a method is provided for directing testing, the method comprising: measuring activities of one or more regions of interest; determining when the measured activities have reached a desired state; and communicating a stimulus to a subject when the measured activities have reached the desired state.

In another embodiment, computer executable software is provided, the software comprising: logic for taking activities of one or more regions of interest and determining when the measured activities have reached a desired state; and logic for causing information to be communicated to a subject regarding when to perform a next behavior when the measured activities have reached the desired state.

In another embodiment, computer executable software is provided, the software comprising: logic for taking measuring activities of one or more regions of interest and determining when the measured activities have reached a desired state; and logic for causing a stimulus to be communicated to a subject when the measured activities have reached the desired state.

In another embodiment, a method is provided for directing testing of one or more regions of interest of a subject, the method comprising: measuring activity in the one or more regions of interest of the subject; determining one or more activity metrics for the measured activity; determining when the one or more activity metrics satisfy a predetermined condition; and communicating a performance reward to the subject; wherein these steps are repeatedly performed in substantially real time. In one variation, the activity metrics measure a similarity between the spatial pattern of activity within the region of interest and a target spatial pattern of activity.

In another embodiment, software is provided for directing testing of one or more regions of interest of a subject, the software comprising: logic for taking measurements of activity of the one or more regions of interest of the subject and determining one or more activity metrics for the measured activity; logic for determining when the one or more activity metrics satisfy a predetermined condition; and logic for causing a performance reward to be communicated to the subject; wherein the software is able to determine the activity metrics from the activity measurements and cause information to be communicated in substantially real time.

In another embodiment, a method is provided for directing testing of one or more regions of interest of a subject, the method comprising: measuring activity in the one or more regions of interest of the subject; determining what information is to be communicated to the subject based, at least in part, on the measured activity; wherein these steps are repeatedly performed in substantially real time. In one variation, the communicated information is a representation of the measured activity. In another variation, the communicated information is an instruction to the subject.

In another embodiment, a method is provided for directing testing of one or more regions of interest of a subject, the method comprising: measuring activity in the one or more regions of interest of the subject; determining one or more activity metrics for the measured activity; determining when the one or more activity metrics satisfy a predetermined condition; and selecting information to be communicated to the subject based on the satisfaction of the predetermined condition. In a preferred embodiment, these steps are continuously performed. In one variation, the communicated information is a representation of the measured activity. In another variation, the communicated information is an instruction to the subject.

In another embodiment, software is provided for directing testing of one or more regions of interest of a subject, the software comprising: logic taking measurements of activity of the one or more regions of interest of the subject and determining what information is to be communicated to the subject based, at least in part, on the measured activity; wherein the software is capable of taking the measurements of activity and determining what information is to be communicated in substantially real time. In one variation, the communicated information is a representation of the measured activity. In another variation, the communicated information is an instruction to the subject.

In another embodiment, software is provided for directing testing of one or more regions of interest of a subject, the software comprising: logic taking measurements of activity of the one or more regions of interest of the subject and determining one or more activity metrics for the measured activity; logic for determining when the one or more activity metrics satisfy a predetermined condition; and logic for selecting information to be communicated to the subject based on the satisfaction of the predetermined condition. In a preferred embodiment, the software is capable of taking the measurements of activity and selecting the information to be communicated in substantially real time.

In another embodiment, a computer assisted method is provided for guiding brain activity testing comprising: measuring activity of one or more regions of interest of a subject; employing computer executable software to determine information to communicate to the subject based, at least in part, on the measured brain activity; and employing computer executable software to communicate the information to the subject.

In another embodiment, a computer assisted method is provided for guiding brain activity testing, the method comprising: measuring activity of one or more regions of interest of a subject; employing computer executable software to determine instructions based, at least in part, on the measured brain activity; and employing computer executable software to communicate the instructions to the subject. In one variation, measuring activity comprises recording activity data from a scanner, converting the recorded activity data to image data, and preprocessing the image data; and communicating the information comprises displaying images derived from the preprocessing image data.

In another embodiment, a method is provided for directing testing of one or more regions of interest of a subject, the method comprising: measuring activity in the one or more regions of interest of the subject; determining how to communicate information to the subject based, at least in part, on the measured activity; wherein these steps are repeatedly performed in substantially real time.

In another embodiment, software is provided for directing testing of one or more regions of interest of a subject, the software comprising: logic taking measurements of activity of the one or more regions of interest of the subject and determining how information is to be communicated to the subject based, at least in part, on the measured activity; wherein the software is capable of taking the measurements of activity and determining how information is to be communicated in substantially real time.

In another embodiment, a method is provided for selectively activating one or more regions of interest, the method comprising: (a) communicating one or more stimuli to a subject and/or having the subject perform one or more behaviors that are directed toward activating the one or more regions of interest without measuring activation of the one or more regions of interest; and (b) communicating the same one or more stimuli to the subject and/or having the subject perform the same behaviors as in step (a) in combination with measuring brain activity in the one or more regions of interest as the subject is exposed to stimuli and/or performs the behaviors. In one variation, information is displayed to the subject in step (a) that simulates the information that is displayed to the subject during step (b).

In another embodiment, software is provided for use in testing, the software comprising logic for communicating information to guide a subject in the performance of a testing exercise during which activation is not measured; and logic for communicating information to guide a subject in the performance of a testing exercise during which activation of one or more regions of interest is measured; wherein information is displayed to the subject when activity is not measured that simulates activity measurements that are displayed when activity is measured.

In another embodiment, a method is provided for selectively activating one or more regions of interest, the method comprising: communicating information to a subject that instructs a subject to perform a sequence of behaviors or have a series of perceptions that are adapted to cause the selective activation of one or more regions of interest.

In another embodiment, a method is provided for selectively activating one or more regions of interest, the method comprising: identifying information that instructs a subject to perform a sequence of behaviors or have a series of perceptions that selectively causes activation of one or more brain regions in a subject; communicating the identified information to a same or different subject; and measuring activation of one or more regions of interest in response to the communicated information.

In another embodiment, software is provided for use in testing, the software comprising logic for communicating information to guide a subject in the performance of a testing exercise during which activation of one or more regions of interest is not measured, the logic displaying information that simulates activity measurements of the one or more regions of interest.

In another embodiment, software and information is provided for use in testing, the software comprising logic for communicating information to guide a subject in the performance of a testing exercise during which activation is not measured, and the information comprising stimuli, instructions, and/or measured information having been determined based in part upon activity in a region of interest during a testing period when activity was measured and communicated to the same or a different subject in substantially real time.

In another embodiment, a method is provided for selecting how to achieve activation of one or more regions of interest, the method comprising: (a) having a subject perform a set of behaviors; (b) measuring how well each of the behaviors in the set activate the one or more regions of interest; (c) selecting a subset of the behaviors from the set found to be effective in activating the one or more regions of interest; and (d) after step (c) and in the absence of measuring activation, determining what information to communicate to the same or a different subject based, at least in part, on the activity measurements of step (b). In one variation, evaluating the set of behaviors comprises calculating and comparing activation metrics computed for each behavior based on measured activities for the different behaviors. In another variation, the behaviors evaluated are overt behaviors involving a physical motion of the body of the subject. In another variation, the behaviors are covert behaviors only cognitive processes which do not lead to a physical motion of the body of the subject. In the case when the subject in step (a) is different than the subject in step (d), the subject in step (d) may have a commonality with the subject of step (a) in relation to the one or more regions of interest upon which the behaviors were selected.

In another embodiment, computer executable logic is provided for selecting how to achieve activation during testing of one or more regions of interest of a subject, the software comprising: logic for calculating activation metrics for activity measured for one or more regions of interest in a first subject; logic for comparing a set of calculated activation metrics and selecting a subset of the activation metrics having a superior activation of the one or more regions of interest in that first subject; logic that takes the measured brain from the first subject and determines for a second subject one or more members of the group consisting of: a) what next stimulus to communicate to the second subject, b) what next behavior to instruct the second subject to perform, c) when the second subject is to be exposed to a next stimulus, d) when the second subject is to perform a next behavior, e) one or more activity metrics computed from the measured activity in the first subject, f) a spatial pattern computed from the measured activity in the first subject, g) a location of a region of interest computed from the measured activity of the first subject, h) performance targets that the second subject is to achieve computed from the measured activity in the first subject, i) a performance measure the second subject's success computed from the measured activity in the first subject; and logic for communicating information based on the determinations to the second subject. In one variation, the information communicated to the second subject is communicated during a process of testing. In another variation, the information communicated to the second subject is a set of instructions and/or stimuli to be used by the second subject in performing testing trials. In another variation, the information communicated to the second subject is a set of instructions and/or stimuli to be used by the second subject in performing testing trials for the activation of a brain region of interest in the second subject.

In another embodiment, computer executable logic is provided for selecting how to achieve activation during testing of one or more regions of interest of a subject, the software comprising: logic for calculating activation metrics for activity measured for one or more regions of interest during each of several behaviors in a first subject; logic for comparing a set of calculated activation metrics corresponding to the set of behaviors and selecting a subset of the activation metrics and their corresponding behaviors having a superior activation of the one or more regions of interest in that first subject; logic that takes the measured brain activity from the first subject and determines information to communicate to a second subject; and logic for communicating the determined information to the second subject. In one variation, the logic communicates the determined information to the first subject in substantially real time relative to when the activity is measured.

In another embodiment, a method is provided for selecting how to achieve activation during testing of one or more regions of interest of a subject, the method comprising: calculating activation metrics for activity measured for one or more regions of interest during each of several behaviors in a first subject; and comparing a set of calculated activation metrics corresponding to the set of behaviors and selecting a first subset of the activation metrics and their corresponding behaviors having a superior activation of the one or more regions of interest in that first subject; at a later time: (a) having a second subject perform a behavior adapted to selectively activate one or more regions of interest in the first subject; and (b) optionally communicating information to the second subject based on the measured brain activity in the first subject; wherein steps (a)-(b) are repeated multiple times, the second subject using the communicated information to guide the second subject in the subsequent performance of the behavior. In one variation, computer executable logic is employed to select the information communicated to the subject. In another variation, computer executable logic is employed to cause the information to be communicated to the second subject. In one variation, the first subject and the second subject are the same subject. In another variation, the first subject and the second subject are different subjects. In the case when the first and the second subject are different subjects, the second subject may additionally have been selected based upon having a condition likely to benefit from similar testing as that received by first subject.

In another embodiment, a computer assisted method is provided for guiding brain activity testing comprising: measuring activity of one or more internal voxels of a brain; employing computer executable logic that takes the measured brain activity and determines one or more members of the group consisting of: a) what next stimulus to communicate to the subject, b) what next behavior to instruct the subject to perform, c) when a subject is to be exposed to a next stimulus, d) when the subject is to perform a next behavior, e) one or more activity metrics computed from the measured activity, f) a spatial pattern computed from the measured activity, g) a location of a region of interest computed from the measured activity, h) performance targets that a subject is to achieve computed from the measured activity, i) a performance measure of a subject's success computed from the measured activity, j) a subject's position relative to an activity measurement instrument; and communicating information based on the determinations to the subject in substantially real time relative to when the activity is measured.

Computer executable software for guiding brain activity testing is also provided that comprises: logic which takes data corresponding to activity measurements of one or more internal voxels of a brain and determines one or more members of the group consisting of: a) what next stimulus to communicate to the subject, b) what next behavior to instruct the subject to perform, c) when a subject is to be exposed to a next stimulus, d) when the subject is to perform a next behavior, e) one or more activity metrics computed from the measured activity, f) a spatial pattern computed from the measured activity, g) a location of a region of interest computed from the measured activity, h) performance targets that a subject is to achieve computed from the measured activity, i) a performance measure of a subject's success computed from the measured activity, j) a subject's position relative to an activity measurement instrument; and logic for communicating information based on the determinations to the subject in substantially real time relative to when the activity is measured.

Computer executable software is also provided for guiding brain activity testing that comprises logic which takes a measurement of brain activity in one or more regions of interest of a subject while the subject has one or more perceptions and/or performs one or more behaviors that are directed toward activating the one or more regions of interest and determines one or more members of the group consisting of a) what next stimulus to expose the subject to, b) what next behavior to have the subject perform, c) what information to communicate to the subject, d) when a subject is exposed to the next stimulus, e) when the subject is to perform the next behavior, f) when new information is to be communicated to the subject, g) how a subject is exposed to the next stimulus, h) how the subject is to perform the next behavior, and i) how new information is to be communicated to the subject. In one variation, the software performs the determinations in substantially real time relative to when the brain activity measurement is taken. In another variation, the determined information is communicated to the subject.

In another embodiment, a method for guiding brain activity testing is provided that comprises: having a subject perform a behavior or be exposed to a stimulus; measuring activity of the one or more regions of interest as the behavior is performed or the subject is exposed to the stimulus; and communicating information to the subject based on the measured brain activity in substantially real time relative to when the behavior is performed or the subject is exposed to the stimulus.

In another embodiment, computer executable software is provided for guiding brain activity testing, the software comprising: logic for instructing a subject to perform a behavior; logic for taking activity measurements of one or more regions of interest as the behavior is performed and communicating information to the subject based on the measured brain activity in substantially real time relative to when the behavior is performed.

In another embodiment, a method is provided for guiding brain activity testing, the method comprising: (a) having a subject perform a behavior adapted to selectively activate one or more regions of interest; (b) measuring activity of the one or more regions of interest as the behavior is performed; and (c) communicating information to the subject based on the measured brain activity in substantially real time relative to when the behavior is performed; wherein steps (a)-(c) are repeated multiple times, the subject using the communicated information to guide the subject in the subsequent performance of the behavior. In one variation, computer executable logic is employed to select the information communicated to the subject. In another variation, computer executable logic is employed to cause the information to be communicated to the subject.

In another embodiment, computer executable software is provided for guiding brain activity testing, the software comprising: logic for taking activity measurements of one or more regions of interest as a behavior is performed; and logic for communicating information to the subject based on the measured brain activity in substantially real time relative to when the behavior is performed; wherein the logic takes new activity measurements as they are received and communicates new information based on the new activity measurements. In one variation, the software is able to take the activity measurements and cause the information to be communicated in substantially real time.

In another variation, the software further includes logic for selecting what information is to be communicated.

In another embodiment, a method is provided for diagnosing a condition of a subject associated with particular activation in one or more regions of interest, the method comprising: having the subject perform a behavior or have a perception adapted to selectively activate one or more regions of interest associated with the condition; measuring activity of the one or more regions of interest as the behavior is performed or the subject has the perception; and diagnosing a condition associated with the one or more regions of interest based on the activity in response to the behavior or perception.

In another embodiment, a computer assisted method is provided for diagnosing a condition of a subject associated with particular activation in one or more regions of interest, the method comprising: having computer executable logic cause instructions to perform a behavior and/or a stimulus be communicated to the subject, the behavior and/or stimulus being adapted to selectively activate one or more regions of interest associated with the condition; having computer executable logic take activity measurements of the one or more regions of interest in response to the behavior and/or stimulus and diagnose whether the condition is present based on the activity response to the behavior and/or stimulus.

In another embodiment, a method is provided for designing a treatment for a condition of a subject, the method comprising: identifying a behavior or stimulus adapted to selectively activate one or more regions of interest associated with a condition to be treated; having the subject perform the selected behavior or exposing the subject to the selected stimulus; measuring activity of the one or more regions of interest as the behavior is performed or the subject is exposed to the stimulus in order to evaluate the effectiveness of the treatment. In one variation, the method further comprises identifying the one or more regions of interest of a subject associated with the condition to be treated.

In another embodiment, computer executable software is provided for designing a treatment for a condition of a subject, the software comprising: logic for identifying a behavior or stimulus adapted to selectively activate one or more regions of interest associated with a condition to be treated; logic for instructing the subject to perform the selected behavior and/or communicating the selected stimulus to the subject; and logic for taking activity measurements of the one or more regions of interest as the behavior is performed or the subject is exposed to the stimulus and evaluating the effectiveness of the treatment. In one variation, the software further comprises logic for identifying the one or more regions of interest of a subject associated with the condition to be treated.

In another embodiment, a method is provided for treating one or more regions of interest of a brain of a subject, the method comprising: having a subject perform a behavior or have a perception adapted to activate one or more regions of interest where the resulting activity of the one or more regions of interest is measured as the behavior is performed or the subject is exposed to the stimulus. In one variation, information selected from the group consisting of instructions, stimuli, physiological measurement related information, and subject performance related information is communicated to the subject as the behavior is performed or the perceptions are being made. In another variation, information selected from the group consisting of instructions, stimuli, physiological measurement related information, and subject performance related information is communicated to the subject as the behavior is performed or the perceptions are being made, the information communicated to the subject is selected based, at least in part, on the measured activity. In one variation, the one or more regions of interest selected are implicated in the etiology of a condition that the subject has. In another variation, the one or more regions of interest selected are related to a disease state. In another variation, the one or more regions of interest selected have an abnormality related to a disease state. In another variation, the one or more regions of interest are adjacent to a region of the brain that has been injured.

In another variation, a method is provided for selecting a brain region of interest, the method comprising: having a subject perform a behavior or have a perception adapted to activate one or more localized regions of the brain; measuring activity of the localized regions of the brain of the subject as the behavior is performed or the perception is made; and identifying one or more localized regions of the brain of the subject whose activation changes in response to the behavior or perception. In one variation, the method further comprises storing a location of the identified one or more regions of interest to memory. In one variation, identifying the one or more localized regions of the brain is performed less than 10, 5, 1, 0.1 minutes after the behavior is performed or the perception is had.

In another variation, computer executable software is provided for selecting a brain region of interest, the software comprising: logic for instructing a subject perform a behavior adapted to activate one or more localized regions of the brain; logic for taking activity measurements of the regions of interest of the subject as the behavior is performed and identifying one or more regions of interest of the subject whose activation changes in response to the behavior or perception. In one variation, the software further comprises logic for selecting coordinates corresponding to the identified one or more regions of interest. In another variation, the software further comprises logic for selecting coordinates corresponding to the identified one or more regions of interest and storing the selected coordinates to memory.

In another embodiment, a method is provided for selecting a brain region of interest, the method comprising: having a subject perform a behavior or have a perception; measuring activity of the regions of interest of the subject as the behavior is performed or the perception is made; and identifying one or more regions of interest of the subject whose activation changes in response to the behavior or perception.

In another embodiment, a computer assisted method is provided for evaluating an effectiveness of brain activity testing comprising: selecting a target level of activation for one or more regions of interest of a subject; having the subject perform a behavior or have a perception; measuring activity of one or more regions of interest of a subject; employing computer executable software to compare the measured activity to the target level of activity. In one variation, the target level of activity is communicated to the subject. In another variation, the target level of activity is displayed to the subject as the subject performs the behavior or has the perception. In yet another variation, the comparison between the measured activity and the target level of activity is communicated to the subject. In yet another variation, the comparison between the measured activity and the target level of activity is displayed to the subject. In yet another variation, the computer executable software selects information to be communicated to the subject based on the comparison between the measured and target levels of activity. In yet another variation, the software selects instructions to be communicated to the subject based on the comparison between the measured and target levels of activity. In yet another variation, the software selects a behavior to be performed or a stimulus to expose the subject to based on the comparison between the measured and target levels of activity. In yet another variation, comparing comprises computing one or more members of the group consisting of a vector difference, a vector distance, and a dot product between two vectorized spatial patterns of physiological activity.

In another embodiment, computer executable software is provided for evaluating an effectiveness of brain activity testing, the software comprising: logic for selecting a target level of activation for one or more regions of interest of a subject; logic for communicating instructions to the subject to perform a behavior and/or communicate a stimulus to the subject; logic for taking activity measurements of one or more regions of interest of a subject and comparing the measured activity to the target level of activity. In one variation, the software comprises logic for communicating the target level of activity to the subject. In another variation, the software comprises logic for causing the target level of activity to be displayed to the subject as the subject performs the behavior or as the stimulus is communicated. In yet another variation, the software comprises logic that communicates the comparison between the measured activity and the target level of activity to the subject. In yet another variation, the software comprises logic for displaying the comparison between the measured activity and the target level of activity to the subject. In yet another variation, the software comprises logic for selecting information to be communicated to the subject based on the comparison between the measured and target levels of activity. In yet another variation, the software comprises logic for selecting instructions to be communicated to the subject based on the comparison between the measured and target levels of activity. In yet another variation, the software comprises logic for selecting a behavior to be performed or a stimulus to communicate to the subject based on the comparison between the measured and target levels of activity. In yet another variation, the logic for comparing comprises logic for computing one or more members of the group consisting of a vector difference, a vector distance, and a dot product between two vectorized spatial patterns of physiological activity.

In another embodiment, a testing method is provided that comprises: having a subject perform a behavior or be exposed to a stimulus; measuring activity of the one or more regions of interest as the behavior is performed or the subject is exposed to the stimulus; and having the subject estimate the measured activity. In one variation, no behavior or stimulus may be used. In another variation, the behavior used is the cognitive process of forming an estimate of measured activity. In one variation, the method further comprises communicating information to the subject regarding how well the subject estimated the measured activity. In another variation, the subject inputs his or her estimate into a system. In another variation, the method further comprises recording to memory how well the subject estimated the measured activity. In another variation, an activity metric is calculated based on the measured activity and the subject estimates the activity metric. It is noted that the subject's estimate of the measured activity can be a qualitative estimate (e.g., higher than a value, lower than a value) or quantitative (e.g., a numerical estimate).

In another embodiment, computer executable software is provided that comprises: logic for taking activity measurements for one or more regions of interest; and logic for receiving a subject's estimate of activation of one or more regions of interest in response to a behavior or perception and comparing that estimate to the measured activation for one or more regions of interest. In one variation, the software further comprises logic for creating a displayable image illustrating the comparison of the subject's estimate. In another variation, the software further comprises logic for communicating information to the subject regarding how well the subject estimated the measured activation. In another variation, the logic stores the estimate and activation measurements to memory. In another variation, the logic calculates an activity metric based on the measured activation. In another variation, the subject's estimate is an estimated activity metric and the logic compares an activity metric based on the measured activation to the subject's estimated activity metric. It is noted that the subject's estimate of the measured activity can be a qualitative estimate (e.g., higher than a value, lower than a value) or quantitative (e.g., a numerical estimate).

Also according to any of the above embodiments, in one variation, measurements are used in diagnosing a condition of the subject.

Also according to any of the above embodiments, in one variation, measurements are used in diagnosing a condition of the subject at two or more time points that are separated by an intervening time. During this intervening time, the subject may remain inside the measurement apparatus. Alternatively, the intervening time may encompass a longer period.

Also according to any of the above embodiments, in one variation, measurements are used the staging of the condition of a subject, or the repeated staging of this condition over the progression of a condition.

Also according to any of the above embodiments, in one variation, measurements are used in the testing of the progression of an intervention at a succession of multiple time points.

Also according to any of the above embodiments, in one variation, measurements are used the testing of the progression of a condition at a succession of multiple time points.

Also according to any of the above embodiments, in one variation, measurements are used in diagnosing a condition of the subject at two or more time points that are separated by an intervening time including an intervention. The change in measurements made at different time points may be used to assess the effects of the intervention. This intervention may comprise a pharmacological treatment, other therapeutic treatment, or training of the subject.

Also according to any of the above embodiments, the measurements may be performed using parallel MRI imaging, acquired with two or more receive coils. In one implementation, this may use the SENSE algorithm for MRI image reconstruction.

Also according to any of the above embodiments, the measurements may be performed using steady state free precession (SSFP) MRI imaging, acquired with two or more receive coils.

Also according to any of the above embodiments, the behavior may optionally be selected from the group consisting of sensory perceptions, detection or discrimination, motor activities, cognitive processes, emotional tasks, and verbal tasks.

Also according to any of the above embodiments, the methods are optionally performed with the measurement apparatus remaining about the subject during the method.

According to any of the above embodiments, in one variation, measuring activation is performed by fMRI.

According to any of the above embodiments, in one variation, the activity measurements are made using an apparatus capable of taking measurements from one or more internal voxels without substantial contamination of the measurements by activity from regions intervening between the internal voxels being measured and where the measurement apparatus collects the data.

Also according to any of the above embodiments, pretraining is optionally performed as part of the method.

Also according to any of the above embodiments, in one variation, at least one of the regions of interest is an internal region of the brain.

Also according to any of the above embodiments, in one variation, the one or more localized regions are all internal relative to a surface of the brain.

Also according to any of the above embodiments, in one variation, the one or more regions of interest comprise a voxel.

Also according to any of the above embodiments, in one variation, the one or more regions of interest comprise a plurality of different voxels.

According to any of the above embodiments, in one variation, the one or voxels measured has a two dimensional area. The two dimensional area optionally has a diameter of 50, 30, 20, 15, 10, 5, 4, 3, 2, 1, 0.5, 0.1 mm or less.

According to any of the above embodiments, in one variation, the one or more voxels measured has a three dimensional volume. The three dimensional volume optionally has a volume of 22×22×12 cm, 11×11×6 cm, 6×6×6 cm, 3×3×3 cm, 1×1×1 cm, 0.5×0.5×0.5 cm, 1×1×1 mm, 100×100×100 microns or less.

Also according to any of the above embodiments, in one variation, measurements are made from at least 100 separate internal voxels, and these measurements are made at a rate of at least once every five seconds.

Also according to any of the above embodiments, in one variation, measurements are made from a set of separate internal voxels corresponding to a scan volume including the entire brain.

According to any of the above embodiments, the one or more regions of interest optionally include one or members of the group consisting of neuromodulatory centers or plasticity centers.

Also according to any of the above embodiments, the methods may be performed in combination with the administration of an agent for enhancing measurement sensitivity of the one or more regions of interest. For example, in one variation, the method is performed in combination with the administration of a fMRI contrast agent. In another variation, the method is performed in combination with the administration of an agent that enhances activity in the one or more regions of interest.

According to any of the above embodiments, measuring brain activity is optionally performed continuously as the subject performs a behavior, has a perception and/or is exposed to a stimulus. For example, measuring brain activity is optionally performed at least every 10, 5, 4, 3, 2, or 1, 0.1, 0.01 seconds or less as the subject performs a behavior, has a perception and/or is exposed to a stimulus.

According to any of the above embodiments, the subjects performs one or more behaviors during measurement that constitute training to activate one or more brain region of interest.

According to any of the above embodiments, the method is used to guide brain activity training by instructing a subject to modulate a brain region of interest.

According to any of the above embodiments, an action is performed in response to a brain activity measurement in substantially real time. For example, an action is optionally performed in response to a brain activity measurement at least every 10, 5, 4, 3, 2, or 1, 0.1, 0.01 seconds or less.

Also according to any of the above embodiments, the behavior is optionally a cognitive task the subject is to perform based on an image displayed to the subject.

Also according to any of the above embodiments, in one variation, communicating information to the subject (for example: instructions, stimuli, physiological measurement related information, and subject performance related information) is performed by one or more of the members selected from the group consisting of providing audio to the subject, providing a smell to the subject, displaying an image to the subject.

Also according to any of the above embodiments, a desired activity metric to be achieved optionally is determined and/or communicated.

Also according to any of the above embodiments, whether a desired activity metric is achieved optionally is determined and/or communicated.

Also according to any of the above embodiments, an activity metric is optionally determined and/or communicated from measured activity. In one variation, the activity metric is modified relative to a baseline level of activation. In another variation, the activity metric is normalized relative to a baseline level of activation. In another variation, a comparison between an activity metric and a reference activity metric is performed.

Also according to any of the above embodiments, a measured activity metric may optionally be determined and/or communicated. In one variation, the activity metric is modified relative to a baseline level of activation. In another variation, the activity metric is normalized relative to a baseline level of activation. In another variation, a comparison between an activity metric and a reference activity metric is performed.

Also according to any of the above embodiments, a measured activation image or volume may optionally be determined and/or communicated. In one variation, the activation image or volume is modified relative to a baseline level of activation. In another variation, the activation image or volume is normalized relative to a baseline level of activation. In another variation, a comparison between an activation image or volume and a reference activation image or volume is performed.

Also according to any of the above embodiments, in one variation, the subject performs a behavior, has a perception and/or is exposed to a stimulus repeatedly for a period of at least 1, 5, 10, 20, 30, 60 or more minutes.

Also according to any of the above embodiments, in one variation, the subject performs a behavior, has a perception and/or is exposed to a stimulus repeatedly at least 2, 3, 4, 5, 10, 20, 100 or more minutes.

Also according to any of the above embodiments, in one variation, activity measurements are recorded to memory during the method. Optionally, activity measurements and the behaviors and/or stimuli used are recorded to memory during the method. Optionally, any information communicated to the subject is also recorded to memory.

Also according to any of the above embodiments, in one variation, activity measurements may be communicated to a remote location. Optionally, activity measurements and the behaviors and/or stimuli used communicated to a remote location during the method. Optionally, any information communicated to the subject is also communicated to a remote location. In one example, this communication to a remote location takes place via internet communication. In another example, this communication to a remote location takes place via wireless communication.

According to any of the above embodiments where information is communicated, in one variation, the information is communicated by a manner selected from the group consisting of providing audio to the subject, providing tactile stimuli to the subject, providing a smell to the subject, displaying an image to the subject.

According to any of the above embodiments wherein information is determined, in one variation, the information is determined while the instrument used for measurement remains positioned about the subject.

Also according to any of the above embodiments wherein information is communicated, in one variation, the information communicated is an instruction to the subject.

Also according to any of the above embodiments wherein information is communicated, in one variation, the instruction is a text or iconic indication denoting an action that a subject is to perform.

Also according to any of the above embodiments wherein information is communicated, in one variation, the instruction identifies a task to be performed by the subject.

Also according to any of the above embodiments wherein information is communicated, in one variation, some of the information communicated to the subject is material to be learned.

Also according to any of the above embodiments wherein an instruction is determined, in one variation, the instruction is determined by computer executable logic.

Also according to any of the above embodiments wherein an instruction is communicated, in one variation, the instruction communicated is selected from a set of instructions stored in memory, the selection being based upon the brain activity measured.

Also according to any of the above embodiments, the subject may optionally input information to the system while brain activity measurements are being taken or while the subject is in a position where brain activity measurements may be taken.

Also according to any of the above embodiments, in one variation, the method further comprises selecting one or more of the internal voxels to correspond to a region of interest for a particular subject and using the selected internal voxels of the region of interest to make the one or more determinations.

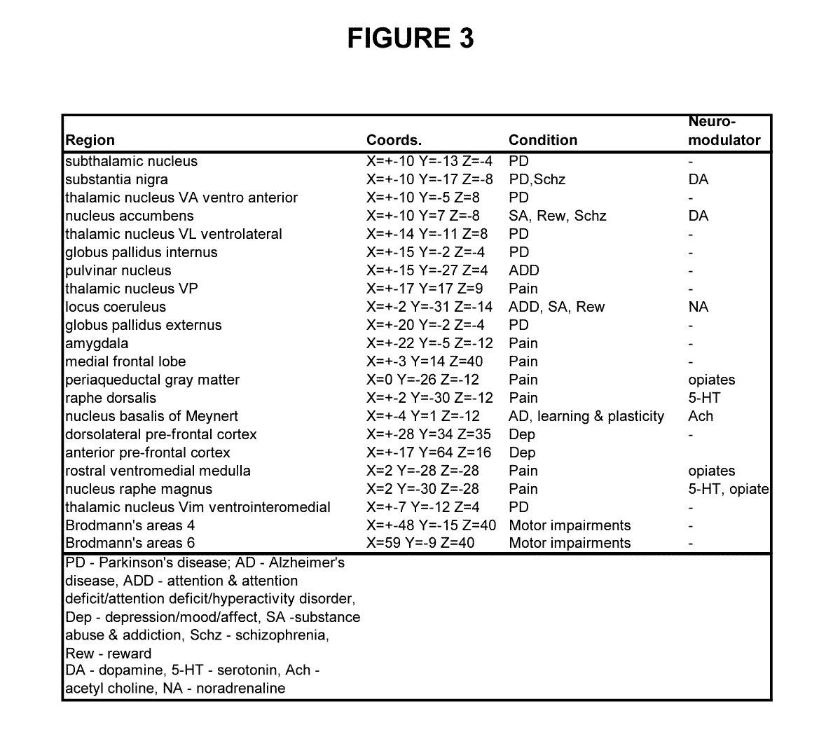

Also according to any of the above embodiments, in one variation, the region of interest is selected from the group consisting of one of the regions listed in FIG. 3, including the substantia nigra, subthalamic nucleus, nucleus accumbens, locus coeruleus, periaqueductal gray matter, nucleus raphe dorsalis, nucleus basalis of Meynert, dorsolateral pre-frontal cortex.

Also according to any of the above embodiments, in one variation, the region of interest has a primary function of releasing a neuromodulatory substance, where the neuromodulatory substance is selected from the group consisting of: dopamine, acetyl choline, noradrenaline, serotonin, an endogenous opiate.

Also according to any of the above embodiments, in one variation, the subject has one or more of the following conditions: Parkinson's disease, Alzheimer's disease, attention & attention deficit disorder, depression, substance abuse & addiction, schizophrenia.

These and other embodiments and variations of the methods, software and systems of the present invention are described herein.

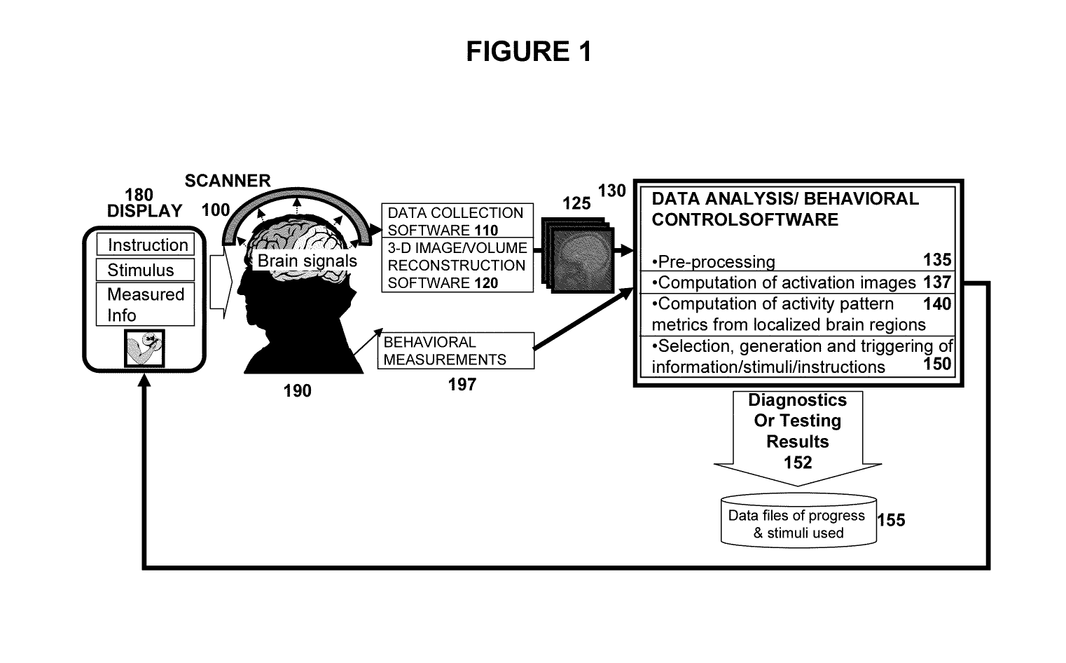

The brain is the seat of psychological, cognitive, emotional, sensory and motoric activities. Many psychological and neurological conditions arise because of inadequate levels of activity or inadequate control over discretely localized regions within the brain. The present invention provides methods, software, and systems that may be used to measure and diagnose the activation and control of one or more regions of interest. An overview diagram depicting the components and process of the invention is presented in FIG. 1. As illustrated, a scanner and associated control software 100 initiates scanning pulse sequences, makes resulting measurements, and communicates electronic signals associated with data collection software 110 that produces raw scan data from the electronic signals. The raw scan data is then converted to image data corresponding to images and volumes of the brain by the 3-D image/volume reconstruction software 120. The resultant images or volume 125 is passed to the data analysis/behavioral control software 130. The data analysis/behavioral control software performs computations on the image data to produce activity metrics that are measures of physiological activity in brain regions of interest. These computations include pre-processing 135, computation of activation image/volumes 137, computation of activity metrics from brain regions of interest 140, and selection, generation, and triggering of stimuli or instructions.

In addition, software may present this information to a user, who may use it in determining a diagnosis, or in testing 152. These results may also be computed using software logic. The results and other information and ongoing collected data may be stored to data files of progress and a record of the stimuli used 155. These results may be used in diagnosis and testing, such as to diagnose the condition of the subject, or test the impact of an agent, such as a pharmacological treatment. A selected instruction, or stimulus, then may be presented via a display means 180 to a subject 190. If the subject undertakes overt behaviors, such as responding to questions, the responses and other behavioral measurements 197 are fed to the data analysis/behavioral control software 130. The instruction may also be to ‘rest’, or not to perform an overt behavior.

As will be explained herein, any brain measurement methodology may be used in conjunction with the present invention so long as the physiological activity of one or more discretely localized regions of the brain can be effectively monitored. In one particularly important embodiment that will be described in greater detail, the brain scanning methodology used is functional magnetic resonance imaging (fMRI).

In one variation, the regions of interest targeted by this invention are internal relative to a surface of the brain. By using brain scanning technology, such as MRI/fMRI that is able to make measurements from internally localized regions of the brain, the present invention is able to treat those internal localized regions of the brain. A major advance in measuring the activity in discretely localized brain regions was the advent of brain scanning technologies, such as fMRI, PET, and SPECT. These technologies overcome the obstacle of measuring the activity in localized regions internal to the brain without substantial contamination from surrounding and intervening tissue. For example, an MRI/fMRI scanner uses a different magnetic field strength at each point in space, which corresponds to a different RF center frequency for measurement. MRI/fMRI is therefore able to make measurements from only a single point (based upon field strength) by recording RF at the relevant center frequency. This measurement is not significantly contaminated by activity from surrounding regions, or be regions between the point being measured and the surface of the brain.

By using brain scanning technology that can accurately measure internal localized regions of the brain, the present invention is able to monitor internal, localized brain regions. The brain is a structure with hundreds of individual regions, some extremely small, and each with its own function. In order to monitor the brain's actions in a meaningful way, it is important to spatially localize which regions are measured, which regions are activated, and which regions are de-activated. This invention allows the monitoring of small, discretely localized brain regions. This invention also allows the monitoring of the pattern of activity within a brain region to measure a 2-D or 3-D pattern of activation that can include sub-regions of increased activation and sub-regions of neutral or decreased activation.

This invention can employ measurements made using a scanning methodology that records data from each point in a predefined volume. In another variation, the localized brain region that is monitoried is as small as a single voxel. Taking measurements from a single point or small volume allows data collection to be concentrated on the single volume of measurement, rather than being divided across multiple measurement points across a larger volume. This also can obviate the need for elements of the technology that enable scanning of the measurement point.

The present invention may be applied to any disease or condition involving inappropriate activity in one or more discretely localized brain region. For example, the present invention can be used to address a decrease in activation of the substantia nigra that leads to a decrease in the release of the endogenous neuromodulator dopamine in Parkinson's disease with resulting changes in activation in target areas, the decreases in activation in areas including and surrounding the area of tissue injury such as in stroke or other brain trauma, the decrease in activation in the nucleus basalis of Meynert that leads to a decrease in the release of the endogenous neuromodulator acetylcholine to regulate the cerebral cortex in Alzheimer's disease, the changes in activation in the pain network (including some of: the anterior cingulated cortex, the insular cortex, the thalamus, the primary or secondary somatosensory cortex, or the periacqueductal gray) that accompany acute or chronic pain, or the decrease in frontal cortical activity in Major Depression that can be positively impacted by increased release of the endogenous neuromodulator serotonin from serotonergic nuclei and correlates with changes in activation in the frontal cortex.

The present invention can also be applied to subject-specific conditions involving a decrease in activity within a particular discretely localized region, such as the decrease in activity in the still-living tissue adjacent to tissue destroyed by ischemic brain injury (CVA/stroke).

The present invention may be applied to any condition involving the nervous system. The present invention is particularly well-suited for conditions that have a cause directly related to an inappropriate level or pattern of neural activation within one or more discretely localized brain regions. This is because the invention utilizes technology that allows these discretely localized brain regions to be directly spatially targeted and measured or diagnosed.

A feature of the methods, software and systems of the present invention is the communication to a subject through visual, auditory or other information, instructions, or stimuli to guide perception or behavior, or to inhibit behavior.

A further feature of the methods, software and systems of the present invention is the identification of certain exercises that can regulate the physiological activity levels of those discretely localized regions of the brain. By first identifying what exercises are most effective for a selected localized portion of a given subject's brain, the localized activation provided by the present invention is enhanced.

By performing the methods of the present invention, levels and patterns of physiological activation can be measured within regions of interest. A partial list of diseases or conditions which may be addressed by the present invention include, but are not limited to Parkinson's disease, Alzheimer's disease, depression, pain, psychosis, epilepsy, dementia, migraine, and others, and those described in: Adams & Victor's Principles Of Neurology by Maurice Victor, Allan H. Ropper, Raymond D. Adams.

Different aspects of the present invention, including more specific methods, software, and systems are provided herein. The following paragraphs provide an overview of an embodiment of testing and exercise according to the invention. Further embodiments and details are provided in the sections that follow.

The detailed discussion that follows through section 6 describes aspects of an embodiment of this invention that allows testing and exercise of a subject for the purpose of treatment or diagnosis of a condition through the regulation of certain brain regions.

1. Determining a Diagnostic Method for a Given Condition

This section describes a process by which diagnostic methods for different conditions may be developed. It is noted that the subjects referred to in this section are not necessarily subjects that are being diagnosed according to the present invention. Instead, the subjects referred to in this section are people who are used to evaluate how well given diagnostic works.

Developing diagnostic methods for different conditions may be performed by evaluating the likely success of the diagnostic for changing the expected likelihood that the subject has the condition, or is within a particular population (such as a population at risk for a condition, or with a particular characteristic). This may involve understanding whether there is an association between a given condition and one or more particular brain regions; determining the one or more regions of interest to be measured for the given condition; determining one or more classes of exercises likely to engage those brain regions or rest; determining one or more types of analysis to be performed on data collected from the regions of interest; optionally comparing the results of these analyses for each subject with the results from a population of previously measured subjects who either had the condition, or did not have the condition, and determining how the results of these analyses will be used to determine the likelihood that a particular subject has a given condition.

A. Evaluating a Likely Effectiveness of Diagnosing a Given Condition

Numerous different conditions may benefit from diagnosis according to the present invention. For example, Parkinson's disease is caused largely by insufficient activity of the brain's substantia nigra, and resultant patterns of activity in its neural target zones. The activity in the substantia nigra and its target zones can be estimated through measurements of the fluctuations of physiological activity in this area either at rest or during a task, or measurements of the relationship between these fluctuations and fluctuations in other brain regions, measurements of the relationship between these fluctuations and a task that is performed. In the case of stroke, regions adjacent to the zone destroyed by ischemia can be diagnosed using similar measurements. In the case of psychiatric conditions, associated brain regions can be diagnosed using similar measurements. Many other examples of conditions that may benefit from testing according to the present invention are described in the Examples section herein.

The likelihood of success for diagnosis of a given condition can be evaluated from knowledge of the etiology and variety of causal factors contributing to the condition as understood at the time of treatment. More specifically, when considering whether diagnosis will be effective for a given condition, attention should be given to whether the condition is related to brain activity. If there is a correlation between the presence of the condition and a level or pattern of brain activity in one or more regions of interest, then, the methods of the present invention are likely capable of diagnosing that condition. Further, the ability of this methods described in this invention to treat a given condition can be evaluated by making measurements on a sample of individuals with the condition, and a sample of individuals without the condition. For any of the activity metrics described below, the distribution of values of the metric for individuals with and without the condition can be assessed for a range of individuals. If there is a separation of the values of the activity metrics between these groups, then the metric can be used to diagnose the presence of the condition. Further, statistical methods can assign a probability of the existence of the condition for an individual with a particular value of an activity metric based upon the observed likelihood of finding members of the groups of individuals with and without the condition that have that value of the activity metric.

B. Determining One or More Regions of Interest to be Tested for the Given Condition

As noted above, the brain comprises thousands of individual regions, each with its own function. Thus, in order to diagnose a given condition, it is important to identify the one or more regions of interest associated with the condition, or a general pattern associated with the condition. It should be noted that the precise location of these regions can vary subject to subject. Hence, it is also important to identify the one or more regions of interest to be measured for a given subject. This ultimately makes the diagnostic methods of the present invention highly individualized.

Determining the one or more discretely localized brain regions to be measured for a given condition may be performed through a combination of general knowledge about what regions are associated with the given condition and thus need to be exercised, and information about the particular subject.

For a given condition, the scientific and clinical literature will typically have information regarding which localized brain regions are associated with the given condition. For example, the literature may have information associated with a given condition regarding human and animal neural lesion data, pathology, histochemistry, pharmacology, brain stimulation studies, neural recording studies, and functional and anatomical imaging studies. Using this information, one is able to take a subject with a given condition, and determine which brain areas are most relevant.

Once brain regions associated with a given condition are identified in the abstract, it is important to then identify these regions in a given subject's brain. It may be advantageous to store information regarding the location of the relevant brain regions for a given once they are identified so that less time and effort is needed to relocate them for subsequent treatments.

In the case of fMRI scans or other brain scanning methods, the regions of interest can either lie within a single plane of section, or they can form contiguous or non-contiguous volumes consisting of regions on multiple planes of a section. Software allows the definition of standard-sized regions of interest, centered on a location selected by the device operator or based upon anatomical boundaries or measured physiological activation patterns. Once particular regions of the brain are identified for a given subject, the regions may be saved numerically to some form of memory (e.g., a computer disk) so they can be recalled for separate scanning runs, or for scans conducted in different sessions at later dates.

C. Determining One or More Classes of Instructions or Stimuli Likely to Engage the Brain Regions of Interest

Different regions of the brain are associated with different functions, and may thereby be engaged and exercised by particular types of stimuli, or by particular behaviors associated with those functions. Hence, by understanding what function a given region of the brain performs, exercises can be designed which activate those brain regions. Through trial and error, exercises can be varied and thereby fine tuned both with regard to their effectiveness in activating a given region in general, and with regard to their effectiveness in activating a given region for a given subject.

Numerous physiological studies on many different brain regions have been performed and have yielded a wealth of information regarding the different kinds of stimuli or behaviors that can be used to engage different specific brain regions. Many areas of the brain have already been ‘mapped’ in their functionality, in that particular zones are activated by particular types of stimuli or behaviors, with adjacent zones activated by similar stimuli or behaviors. These types of studies have allowed for the determination of what classes of stimulus or behavior are likely to activate particular brain regions by selecting the stimulus or behavior that are appropriate to the type of map and the point on the map being considered.

For example, countless detailed studies have determined frontal cortical regions that subserve movements, the motor cortex. Thus, a lesion that partially inactivates the cortical hand representation will destroy tissue engaged in hand movements. Adjacent tissue will be involved with the other hand, wrist, and arm movements. Therefore, in order to diagnose tissue involve in or spared by the lesion, exercises to employ will include exercises that engage the brain region where the lesion is located as well as adjacent regions. In this instance, such exercises will likely encompass movements of the relevant extremity, whether physically or mental thoughts of their movement.

In some cases, the appropriate behavior for measurement of brain activity may be rest. This may be particularly appropriate in instances when a stimulus or behavior that activates a region of interest is not know, in cases where the subject cannot or will not perform a task or observe a stimulus that activates a region of interest, or in cases where communication with the subject is impaired, such as in small children or patients with dementia.

2. Pre-Training a Subject

Once a diagnostic method has been determined for a particular condition, as described in the preceding section, subjects may be diagnosed for their likelihood of having that condition. Prior to diagnosis, it is advantageous in some subjects to first evaluate whether a particular subject is suitable for diagnosis based upon defined selection criteria; explain the diagnosis process in detail to the subject; and then pre-train the subject using a simulated training environment.

A. Defining Subject Selection Criteria and Screening Subjects

It is desirable for the diagnosis of the present invention to have a high frequency of success. It is therefore desirable to select subjects based upon the likelihood of their diagnosis being successful.

Examples of selection criteria that may be used include but are not limited to:
1) Whether the subject has other indicators of having the condition for which diagnosis is intended, based upon standard diagnostic criteria.
2) Whether the subject has other, preferable diagnostic options available.
3) Whether the subject has sufficient cognitive ability to participate in the planned diagnosis.
4) Whether the subject has any contraindication for brain scanning, such as phobias relating to being inside a scanner, or in-dwelling metal objects such as a pace-maker, or movement disorders that would hinder the ability to make prolonged, stationary brain scans.
5) Any indicators predictive of diagnostic success, such as previous success of the method with subjects that are similar based upon diagnostic group or other signs and symptoms.

Each potential subject may be screened based upon some or all of these selection criteria to determine their suitability for diagnosis.

B. Subject Pretraining

It may be advantageous to explain the testing process to the subject before diagnosis takes place in combination with a brain scanner to measure brain activity. Optionally, the subject is pre-trained using a device that simulates the experiences that the subject will experience when actual diagnosis is performed. This may include providing the subject with the same or similar visual and auditory experiences that will later be provided. For example, when graphical interfaces are to be employed, it may be desirable to pretrain a subject using those graphical interfaces, or at least show the subject the graphical interfaces he or she will see and explain their components.

The details and purpose of the training are explained to the subject to allow him or her to be intimately familiar with what he or she will be doing. A number of issues may be explained including: that the goal of diagnosis is to measure activity in their brain while they perform certain behaviors; the importance of being still during the scanning session; the importance of behaving in a similar way each trial and avoiding excessive physiological activity such as deep sighs so that measurements are consistent; the types of exercises that are likely to succeed in activating the brain region of interest.

A subject may also be given detailed descriptions and explanations of the functioning of the brain regions of interest; of the measurement technology being used; of the timecourse of physiological activity changes; of how to communicate with the controller; and so on.

A subject is preferably pre-trained using exercises that closely mimic the exercises that will be performed when the brain activity is being measured. This allows the subject to become familiar with and practiced on the exercises that he or she will be completing. In addition to ensuring that the subject has a clear understanding of what he or she is to do, this allows any habituation of neural responses to the task activities or other early learning effects to approach steady-state. In cases where habituation is not desirable, such as cases where high initial activity gives more robust measurements, this may be omitted.

A subject may also be trained using a simulation device that mimics the user interface and training schedule and uses the same selected stimuli that a subject would encounter during testing in the scanning apparatus. This interface and its functioning will be described in detail below.

Overall, pre-training is typically preferably designed to generate an experience as close as possible to the real training that the subject will undergo. Therefore, the tasks that the subject is asked to perform, the percent correct achieved, the displays that are provided, stimuli that the subject experiences, and actions that the subject undertakes are all preferably similar to those the subject will observe when actual testing is performed.

3. Initial Brain Scanning Setup and Performing Scanning

Before beginning training using this invention, a number of aspects of the invention must be prepared for use. These include preparing the graphical user interface if one is being used, preparing the subject within the scanning apparatus, and setting up for anatomical and physiological scanning. Section 3 lays out many of the aspects of what the invention does in general, while describing the setup of the various components. In particular, it describes all of the computations that we can make, and the displays that we can generate. Later sections then tell us what we actually DO in testing and diagnosis, and give detailed examples of the computations and displays.

A. Preparation for Brain Scanning

Once a subject has been pre-trained, the subject may be introduced into a scanning apparatus where measurements of brain activity are taken and the location of targeted localized regions of the brain are identified. This section describes this process in regard to a magnetic resonance imaging scanner, such as a GE 3.0T Signa MRI scanner. How to perform analogous scanning using other instruments would be understood by one of ordinary skill in the art.

i. Preparation of Subject within the Scanning Equipment

In order to take measurements of localized region of the brain, the subject of course has to be properly positioned relative to the scanner. Placement is made to ensure standard positioning, to help ensure that the subject has a positive and comfortable experience, and to ensure that the subject has access to visual and other stimuli as well as output devices. The subject is ‘landmarked’ by measuring the position of the nasion (bridge of the nose) using the scanner and setting this to a standard zero position, from which measurements will be taken. The subject's head is placed within a coil, such as a dedicated head coil. The coil is selected to give the best signal from the region of interest. The subject is given earplugs or sound cancelling headphones to decrease noise within the scanner. Communication equipment may also be setup between the subject and the device operator or other healthcare professionals in attendance.

ii. Head Motion Stabilization and Physiological Gating

As would be expected, it is desirable that the subject's head remain perfectly stationary. In order to decrease head motion, the subject may be placed within an adjustable or custom-made head motion stabilizer that is secured to the scanner. If additional motion stabilization is required, motion stabilization software may be used to correct data volumes collected for movements of the subject within the scanner. An example of this software is described in CC Lee, et al. Real-time adaptive motion correction in functional MRI. Magn Reson Med 1996;36:536-444. In instances where a structure is being measured that is subject to significant physiological motion, the timing of initiation of successive measurements may also be triggered to correspond with a particular phase of the cardiac or respiratory cycle according to standard methods described in the literature. Also, navigator echo methods may be employed which use echo pulses to locate the physical structures of the brain, and then prescribe further measurement pulses based upon this localization so that the resultant measurements are taken from substantially the same place for each successive measurement. In this instance, before each scan sequence, or before each individual scan, a small amount of data is collected that allows localization of the head (such as anatomical information in 3 planes of section), this is then aligned with the desired position of the subject within the scanner using high speed alignment software as has been described in the literature, and then the difference in position between current values and the desired position is used to prescribe scanning pulses and gradients to allow collection of data from substantially standardized positions each time.

iii. Brain Volume Registration

In order for the position of the head and the related measurements to be comparable from session to session, images and volumes should be registered, allowing precise correspondence of voxels across scans. This volume registration can have a manual component and an automated component. In the manual component, the subject is positioned within the scanner in a stereotyped way to try to achieve similar placement on successive occasions using a bitebar and fixed points of reference within the scanning apparatus. Additionally, the zero point for scanning may set to the nasion of the subject (bridge of the nose) using a standard light beam approach built into the scanner. Finally, scanning sections are prescribed relative to fixed anatomical landmarks within the subject, including but not restricted to the anterior commissure, the posterior commissure, the mid-saggital line, the central sulcus, the temporal pole, the calcarine fissure and pole, and the topmost point on the cerebral cortex. If sections are prescribed in three dimensions based upon the accurate positions of at least three anatomical landmarks on the subject, then the positions of brain regions can be reliably reproduced on successive sessions. Scanning sections can also be prescribed relative to fiducial marks placed on the subject using material opaque to a scanning instrument. If these marks are placed on known locations on the subject, then they can serve as landmarks for scanning.

B. Anatomical Scanning

Anatomical scans of the subject may be made using an imaging apparatus to visualize internal brain structures.

In one embodiment, detailed anatomical images are collected using an MRI scanner. In one particular example, whole-brain imaging data are acquired on a 3 Tesla MRI Signa LX Horizon Echospeed scanner (General Electric Medical Systems, 8.2.5 system revisions) as described in the operating instructions for that instrument. For example, T1 and/or T2 weighted anatomical image data are collected from axial slices through the head which will be in substantial register with physiological data collected later. An embodiment collects 17 axial slices of 7 mm slice thickness, with each slice having a 256×256 voxel resolution over a 22 cm×22 cm area, producing 256×256×17 voxel brain volume data. Higher resolution data may be collected as well, such as 3D T1, T2, or T2* volume data collected at 1 mm×1 mm×1 mm resolution, to allow more detailed anatomical localization by changing the number of voxels in each of the three dimensions. MRI anatomical scanning methods are described in detail in neuroanatomical texts.

C. Physiological Scanning

An aspect of the present invention relates to the performance of brain scanning such that the physiological activity of regions of interest of the brain can be measured and monitored.

i. Measurements

Physiological activity measurement may take one or more of several forms, including fMRI BOLD signals, fMRI EPI signals, PET or SPECT signals, electrical impedance tomographic measurements, magnetic inductance tomographic measurements, electron paramagnetic resonance imaging measurements, capacitance tomography, magnetization transfer imaging, perfusion imaging, diffusion imaging, diffusion tensor imaging, event-related signals conditioned on sensory events/motor behaviors such as event-related potential imaging, or other physiological measurements. These measurements may be made using a variety of physiological recording apparatus. Examples of measurement apparati that may be used alone or in combination include, but are not limited to functional magnetic resonance imaging (fMRI), PET, SPECT, EEG (electroencephalogram) recordings or event-related electrical potentials, MEG recordings (magnetoencephalogram), electrode-based electrophysiological recording methods including single-unit, multi-unit, field potential or evoked potential recording, infrared or ultrasound based imaging methods, or other means of measuring physiological states and processes, and light-based methods such as near-infrared spectroscopy (NIRS) and precisely-timed light measurements such as EROS.

Functional magnetic resonance imaging (fMRI) is a particular example of a brain scanning technology that is capable of measuring and monitoring brain activity. fMRI is based upon changes in Blood Oxygen Level Dependent (BOLD) contrast and provides spatially and temporally resolved visualization of the hemodynamic response evoked by neuronal activation. fMRI scanning can be performed according to widely published procedures. This technique has been described in detail elsewhere including for example in Annu. Rev. Biomed. Eng. (2000) 2:633-660, the references included therein, and An Introduction to Functional Magnetic Resonance Imaging: Principles and Techniques by Richard B. Buxton (Hardcover—November 2001) and Functional MRI—An Introduction to Methods edited by Jezzard, P, Matthews P M, and Smith, S M, Oxford University Press 2001.

In one particular example, whole-brain imaging data may be acquired on a 3 Tesla MRI Signa LX Horizon Echospeed scanner (General Electric Medical Systems, 8.2.5 system revisions) as described in the operating instructions for that instrument. Functional images may be acquired in the same slices as previously collected anatomical images (see above) using T2*-sensitive gradient echo spiral pulse sequence (30 ms TE; 1000 ms TR; 70 degree flip angle; 22-cm FOV; 64×64 acquisition matrix or similar parameters). See for example: Neuroimaging at 1.5 T and 3.0 T: comparison of oxygenation-sensitive magnetic resonance imaging. G. Kr [symbol] ger A. Kastrup G. H. Glover, Magn Reson Med. April, 2001; 45(4):595-604; Three-dimensional spiral fMRI technique: a comparison with 2D spiral acquisition. S. Lai G. H. Glover, Magn Reson Med. January, 1998; 39(1):68-78. The physiological images collected are registered with previously acquired anatomical images by lining the images up voxel-for-voxel. A more thorough fMRI scanning protocol is provided in Section 7 in the Examples.

It is noted that although many of the more detailed descriptions provided herein are directed to fMRI, it should be understood that the present invention may be used with any brain activity measurement technology that is capable of detecting activity in discretely localized brain regions. Over time, it is anticipated that new techniques will be developed with the ability to detect activity in discretely localized brain regions. Furthermore existing measurement technologies may be adapted for detecting activity in discretely localized brain regions. All such measurement technologies, and their combinations, are intended to be employable in conjunction with the present invention.

Once the scanning equipment is setup, physiological activation of the brain is measured. Generally, the process may comprise collecting scan data repeatedly (e.g. continuous collection at one scan per second), reconstructing the raw physiological data into image data in substantially real time, and performing computations on the resultant images as depicted in FIG. 1.

Activity patterns may be measured within regions of interest or for the whole brain, either at a point in time or continuously. This is achieved by scanning the imaging technology sequentially over a number of voxels with some sampling rate, taking measurements from each one. This gives indications of the level of physiological activity at each location at each point in time.

The number of different points that may be monitored will typically decrease as the sampling rate is increased once the operational limits of the equipment is reached. Therefore, it is frequently necessary to specify the locations and sizes (in three dimensions) of the regions of interest to be monitored, as well as the rate at which these regions of interest are to be sampled. These regions of interest may form either a large and contiguous array (such as a cube containing a large number of contiguous voxels), or a number of discrete locations that are one or more voxel in size. The measured values used for the regions of interest can involve time or spatial averaging or other mathematical smoothing of data over a range of samples. In this way, a vector of data may be acquired at each time point, and a larger vector consisting of a time series of data may be collected.

In order to collect scan data, the functional scanning parameters are input. Preferably, the parameters are pre-set, for example using control software incorporated into the instrument. Aside from inputting the functional scanning parameter, other things to check prior to initiating scanning include: informing the subject that the scan is about to begin, insuring that there is adequate data storage space available, and checking that all computer linkages are active.

ii. Scan Voxels, Scan Volumes, and Regions of Interest

As described in the definitions, a voxel refers to a point or three dimensional volume from which one or more measurements are made. Using a suitable scanning methodology, measurements may be collected from a large number of voxels. For example, measurements may be made from each component of a square grid volume of voxels corresponding to a scan volume. This scan volume may be positioned to include some or all of the brain of a subject. In this way, measurements may be made that span the entire brain, or a portion of the brain. Measurements may be made for each voxel in the scan volume at every measurement time. Measurements may be repeated, such as once per second or at other sampling rates. This may produce a full volume image of the activity level of each point in the brain each second.

In many instances, analyses according to present invention are based on a particular subset of volumes from among the entire scan volume. The particular subset of volumes may be theregion of interest for that analysis.

A region of interest may include a selected one or more of the voxels or measurement points. A region of interest may have a spatial shape and extent defined by the voxels that it includes within the entire scan volume. A typical region of interest may be a 5×5 voxel square array, or a 5×5×5 voxel cubic volume, centered on a selected voxel. A process for selecting a region of interest is described in section 4. Since a region of interest may be comprised of multiple voxels from which independent activity measures are made, it may be possible to measure either an aggregate average level of activity from the entire region of interest, or a spatial pattern of activity comprising the activity at each voxel within the region of interest.

Measurement data may also be collected from a single voxel. In the case of collection of data from a single voxel, the one voxel may correspond to the region of interest.

D. Processing of Scan Data into Images and Activity Metrics

FIG. 1 illustrates the process flow diagram for taking raw scan data and producing information that may be communicated to the subject. As illustrated in FIG. 1, raw scan data is converted to image/volume data 125 corresponding to images and volumes of the brain by 3-D image/volume reconstruction software 120. These are referred to as image/volume data, or as images/volumes, to connote the fact that either a single planar image may be used, or a 3-D volume may be used. One of the simplest types of vector representation of physiological activation for the images is a planar section of fMRI activity, taken with some temporal resolution, and some spatial resolution. This provides a single slice image of the state of activation of the brain at a particular instant.

The resulting image/volume data 125 can then be used by the data analysis/behavioral control software 130, which is described in more detail herein. The data analysis/behavioral control software 130 generates information and selects stimuli or instructions to communicate to a subject 190 to influence how the subject performs exercises. This takes place via three steps, each serving to generate the input to the next: 1) pre-processing of data, 2) computation of activation image/volumes, 3) computation of activity metrics.

All of the computed values, such as those described in this section, may be stored to computer memory or a computer storage device for later retrieval. This storage may take place each time computations for a given measurement time point are completed, or it may take place at the end of a trial, or at the end of a block or session. In addition, all of the computed values may be transmitted via the internet or other communication means at the time of computation, or at a later time.

The process illustrated in FIG. 1 will now be described in relation to processing fMRI data. It is noted that analogous data processing may be performed for other data from other types of instrumentation. Detailed examples of processing that may be performed are provided in Examples section 1.

i. Scanner Software and Pulse Sequences

Commercial data collection software 110 is available and typically included with an MRI/fMRI scanner to control the process of initiating scanning pulse sequences, collecting measurements, communicating electronic signals associated with a scan, and producing raw scan data from the electronic signals. The raw data may be in the form of a k-space representation that can be accessed either from computer memory or from a disk file. This representation must be reconstructed to produce a spatial representation of the signal, such as a scan image or volume.

The scanner software includes pulse sequences for RF energy that will be emitted, with the resultant energy emitted from the subject subsequently being measured in the presence of magnetic fields including a static BO field, and gradient and shim fields. This process will be familiar to one skilled in the art. Further details of pulse sequences are presented in the examples section.

ii. Reconstruction Software

Once the output raw data is formed from the data collection software 110, this data serves as the input to the 3-D image/volume reconstruction software 120. The 3-D image/volume reconstruction software 120 performs computations upon this input that result in the output of 2-D scan images or 3-D scan volumes.

Converting the data to 2-D and 3-D scan images may be performed using reconstruction software that performs k-space to volume reconstruction. The reconstruction software 120 can take several forms, which are publicly described and available. MR image reconstruction may use echo planar imaging, spiral imaging, spiral in, spiral out, spiral in/out, or radial methods.

In one embodiment, the resulting output image files from the transformations are flat, header-less files containing 64×64×17 2 byte integers corresponding to values for the voxels for each scan volume. The output image/volume data from the reconstruction software is then passed as one input to the analysis and control software.

iii. Pre-Processing of Image/Volume Data

One function that the data analysis/behavioral control software 130 may perform is to pre-process 135 the input data. It is noted that the software may optionally process the input data without preprocessing.

Once optionally pre-processed, the data may be used to compute activity metrics from image or volume data. These activity metrics may then be used to generate information to present to the subject, and make selections of stimuli or instructions.

The output images generated by the 3-D image/volume reconstruction software 120 are typically transferred to a separate computer that contains the data analysis/behavioral control software 130. The format of these data are transformed if necessary to allow compatibility between computers, and they are read into memory by the data analysis/behavioral control software 130 on the substantially real time control computer in substantially real time. This process can also take place on a single computer if it has sufficient processing power.

Many types of pre-processing of image/volume data are available, and examples are described in detail in Examples section 1.A. As one example embodiment, the images may be motion-corrected, spatially smoothed by convolving each image with a 2-D gaussian filter with a 1 pixel half width. The output of the pre-processing step is an image or volume of pre-processed data at every data collection time. This is similar in form to the input to this step, but transformed by the pre-processing computations.

iv. Computation of Activation Images/Volumes

Taking the images/volumes as input, optionally after they have been pre-processed, the next step is to compute activation images/volumes. This is typically performed by the data analysis/behavioral control software 130. Many types of activation images/volumes can be computed, and examples are described in detail in Examples section 1.B. below. These activation images/volumes can be used first to determine the location of a region of interest for a particular subject, and later as the input for making measurements from this region of interest.

An example activation volume that may be computed for the purpose of determining the location of the region of interest in a subject is a variance image, computed taking preprocessed scan volumes as input by measuring the variance and each voxel over a measurement time period.

v. Computation of Activity Metrics

Once activation images/volumes have been computed, it is possible to use these as inputs to the computation of activity metrics. This process involves computations of values from a defined region on the activation images/volumes that have been measured. Many types of activity metrics can be computed, and examples are described in detail in Examples section 1.C. below. For example, an average value of the variance for all of the voxels within a region of interest may be computed. In this case, the variance data for each voxel in a defined region of interest at each time point are used as input, and an average value of the variance is calculated for each time point for the group of voxels. This average may then be displayed using a graphical user interface.

E. Setup of Graphical User Interface

An important aspect of the present invention relates to employing measured brain activity to provide measured information. Methods of display will be presented; others will be appreciated by one of ordinary skill.

One primary type of display that may be presented include measures of physiological activity such as variance maps, activation maps of the subject's brain activity, activity metrics from localized brain regions. The setup of the user interface and its potential components are described in the following sections.

i. Presenting an Overall User Interface to the Subject and Device Operator

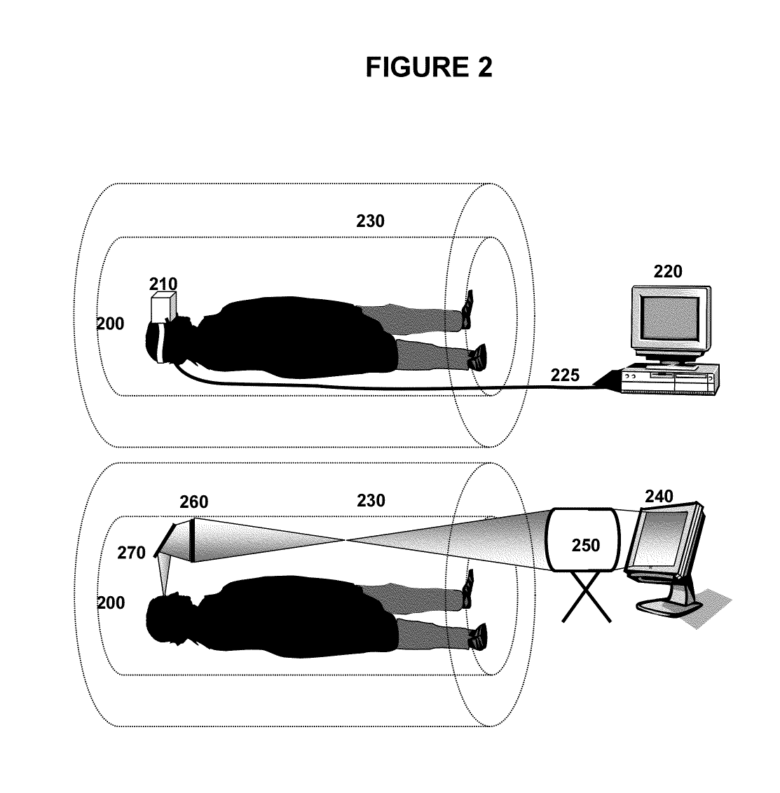

In one embodiment, as shown in FIG. 2, a subject 200 views information such as stimuli, or instructions using viewing goggles 210, such as virtual reality goggles, controlled by a computer 220 connected by a cable 225, while the subject is inside the bore of a scanning apparatus 230. Viewing goggles for the purpose are manufactured by Resonance Technology, Inc, California. The device operator may view a similar screen on a second display. In addition, a remote participant may view a similar display on a remote display screen. Information for remote displaying may be conveyed electronically, for example using a wire, wireless, or interne connection. The display presented for the device operator may be separately configurable to contain a different set of panels than that displayed to the subject.

In another embodiment, the subject 200, views and image displayed on a display 240 and projected through a lens 250 onto a reverse-projection screen 260. The subject views the screen through a mirror 270. Using some form of display, the subject views instructions of what the subject is to do, and/or other forms of information such as perceptual stimuli.

A variety of types of information and display screens can be presented. For example, visual stimuli may be presented to the subject via some form of display. FIG. 2 illustrates one such display system. The types of information that may be displayed are described below after the information that they will contain has been described.

Auditory stimuli may also be presented to the subject, such as digitized speech, tones, music, or other types of sounds. Auditory stimuli may be presented to the subject via some form of speaker system, optionally worn by the subject. Tactile stimuli may be presented using a tactile stimulation apparatus such as a Chubbock stimulator or other tactile stimulator as described in: A tactile air stimulator for humans. E. W. Wineman, Psychophysiology. November, 1971; 8(6):787-9. Temperature stimuli may be presented using skin heating or cooling probes. Olfactory stimuli may be communicated using a device designed to present gaseous odors to the subject in the scanner, as for example described in: Time course of odorant-induced activation in the human primary olfactory cortex. N. Sobel V. Prabhakaran Z. Zhao J. E. Desmond G. H. Glover E. V. Sullivan J. D. Gabrieli J. Neurophysiol. January, 2000; 83(1):537-51. When the subject receives any of these stimuli, associated changes in the brain of the subject may be observed. These changes may then be measured as has been described.

ii. User Interface Screens

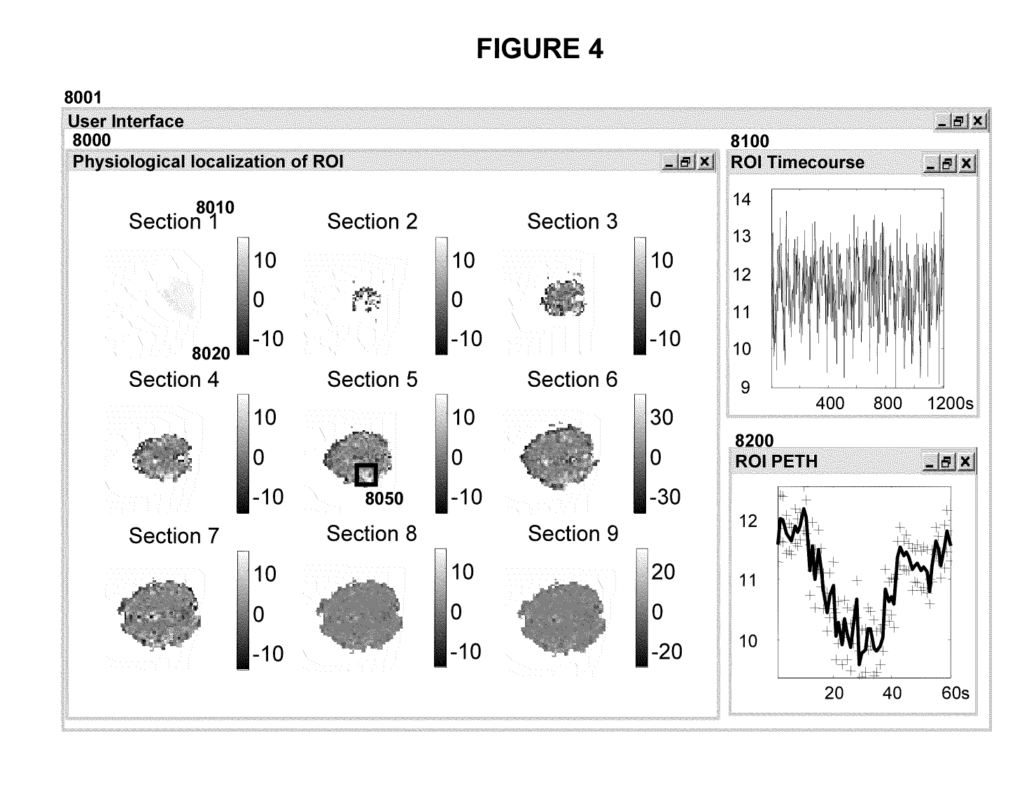

The subject or device operator may view a display a screen 8001 depicted in FIG. 4. This screen may contain a large variety of elements that can be selected for display, or hidden from view, and may each be appropriately sized to be visible in adequate detail. The screen may contain a selector panel that contains a list or set of graphical icons representing the other panels that may be displayed. The user is able to make selections from this selector panel using a pointing device such as a mouse. When a panel has been selected, it becomes visible on the screen, and the subject or device operator can use the pointing device to select the position and size of the panel on the screen. The user may select one or more of each type of panel to display. In some cases, the same type of panel may be displayed more than once for different purposes, such as the use of two anatomy panels, one to show a coronal section, and one an axial section.

iii. Presenting Images and Information

Data obtained and processed from an fMRI or another physiological activity measurement apparatus may be presented to the device operator, and/or another professional that is present, such as a doctor, nurse, technician.

The information displayed can include anatomical brain images, as well as physiological activation images/volumes, and activity metrics. The results of all of the computations described in section 3.D. above may be used as input to present image and metric data. One skilled in the art will recognize possible modes of display for each of the types of computed information described.

4. Localizing Brain Regions of Interest in a Subject

In order to select the area on which measurements may be focused, different methods may be used to localize a region of interest. These methods include anatomical methods for localizing structures, and physiological methods for determining volume activated by a given stimulus or behavior. A region of interest normally corresponds to a subset of the full scan volume that may be collected at each measurement time point. These voxels are selected because of their importance in measurement. The voxels within a region of interest may be defined in a number of ways. They may be defined to be within the anatomical boundaries of one or more brain regions as determined through anatomical scans. They may be defined by the fact that they are activated in correlation with a stimulus, behavior or task. They may be defined arbitrarily by the device operator using a selection screen that allows the device operator to select individual voxels or regions of interest. They may be defined stereotaxically or by adjusting the position of the patient within the measurement apparatus in such a way that the apparatus measures activation from a defined point or area within the subject. The primary region of interest is normally the area that is being tested, and that the subject is attempting to modulate activation within. Comparison regions of interest are other defined regions that may be compared with the primary region of interest, such as other parts of the brain that are not intended to be activated by the task. A region of interest or volume of interest need not be spatially contiguous. For instance, a region of interest might constitute the substantia nigra and sub-thalamic nucleus on both sides of the brain, four non-spatially-contiguous volumes.

A. Anatomical Localization of Brain Regions of Interest

Once anatomical data has been collected for a subject, anatomically defined brain regions may be localized for the subject with reference to the collected anatomical information using either reference to a standard anatomical atlas, or using a manual search. In either case, positions are measured relative to brain landmarks such as the anterior and posterior commissures, and/or fiducial marks placed on defined locations on the subject using scanner-opaque materials.

To use manual search for a structure, the operator can view sections through the 3-D voxel data and search for known brain anatomical structures using radiological knowledge to locate the desired brain regions. The operator can then select combinations of individual voxels using a pointing device, or areas using a bounding line or shape. These selected voxels can be saved in computer memory, as well as saved to disk memory and recalled on later occasions.

Preferably, the software used in combination with the brain imaging device converts the anatomical data to a form that may be displayed or otherwise communicated to the subject or device operator in substantially real time, preferably while the subject is within the scanner. This allows the subject or device operator to use this information to select regions of interest for testing, or to influence how the subject is performing his or her exercises.

In one variation, software is employed that makes a 3-D transformation from standard space to the space of the subject's brain, and back, in substantially real time. For example, the software may take as input a set of 3-D Talairach coordinates or an anatomical volume directly from a computer-generated brain atlas and spatially transform the coordinates according to a 3-D spatial mapping to yield the corresponding locations within the anatomical volume measured for the subject.

Another example of defining a region of interest anatomically is to use a defined anatomical region from a reference brain such as in Talairach or MNI (Montreal Neurological Institute) coordinates. In this case, the anatomical region is defined in the standard coordinates, and then spatially transformed to localize the voxels corresponding to the anatomical structure in the subject's brain. This process is described in further detail at Section 23D in the Examples.

B. Physiological Localization of Brain Regions of Interest

The one or more discretely localized regions of the brain that will define the region of interest that may be used for testing may be defined physiologically through finding the voxels that are modulated by one or more stimulus or behavior in comparison with a background condition. In order to do this, an important aspect of the present invention is its ability to monitor physiological activity in substantially real time after the stimulus or instruction for a behavior is provided so that the effect that the stimulus or behavior had on activity can be accurately determined. In addition, the brain region of interest may be determined within a short period of time after the collection of the physiological data. This short period of time may be less than 10, 5, 2, 1, 0.5, 0.25, 0.01 or less minutes.

Defining the region of interest may be performed by having the subject take part in a set of physiological ROI localization trials. During these trials, the subject engages in behaviors or experiences stimuli that are intended to activate one or more region(s) of interest. By monitoring resultant physiological activity, the location of these one or more region(s) are identified for that subject. The region of interest is normally defined after the completion of these trials based upon the voxels that are modulated. However, it is also possible to define the region of interest before all of the trials are complete, and then iteratively redefine the region of interest as additional substantially real time based measurements are taken.

Regions or volumes of interest may be defined that are modulated by the stimulus or behavior condition, and this determination can be made while the subject is inside the scanning apparatus. Regions of interest may either be defined on a voxel-by-voxel basis, or by defining a circumscribed area or volume such as a rectangle, circle, cube, or spheroid. The defining characteristic for whether each voxel will be within a region of interest may be based upon the value of an activation image/volume at the corresponding voxel. If the voxel is above a defined threshold in the activation image/volume, then the voxel is included in the region of interest. This process can take place either manually, or in a fully or partially automated fashion as described in the following two sections.

i. Example of Presentation of Physiological Localization Trials

The following example illustrates how a physiological localization trial may be performed. It should be noted that the particular physiological localization trial to be used will vary with the subject, the condition to be addressed, and hence the regions of the brain implicated.

In this example, in order to measure the modulation, a stimulus or behavior condition is presented to the subject following a rest or background period to constitute a physiological localization trial. These trials may be repeated one or more times. Measurements are made of the resultant physiological activation patterns in the brain scan volume at multiple time points throughout the localization trials. In order to localize the primary motor cortical representation of the hand, a subject may be asked to alternate between 30 second periods of rest with 30 second periods of moving, or imagining moving, the index finger of the right hand while scanning of the T2* weighted activation level is measured at every voxel within a brain scan volume every second.

ii. Manual Physiological Definition of Region of Interest

Once data has been collected, a region of interest may be determined from physiological localization trials, one or more regions within the brain that are selectively activated during one portion of the trials may be determined. For example, if the trials contain a rest period and a task period, a region may be determined which is activated selectively during the task period compared to the rest period. This process may take place using a principally manual method whereby the subject or device operator selects groups of voxels with strong modulation, any may view data corresponding to the time course of activation of these selected groups of voxels. Alternatively, this process may be partially or fully automated, with software selecting a set of voxels that meet certain criteria, such as a threshold level of modulation.

A wide variety of different physiological activation maps may be computed, as described in section 3.D. In one example, these physiological activation maps may then be used to compute regions of interest through a manual process of selecting the voxels that are activated by a portion of a trial using a provided display screen. For example, the average value during the stimulus or behavior condition minus the average value during the background or rest condition may be computed for each voxel in a scan volume. A montage for the physiological localization of an ROI 8000 using color coded activation maps may be presented to the subject as depicted in FIG. 4 on the user interface 8001. This figure represents actual data collected from a subject in substantially real time, collected using a task involving mental rehearsal of an imagined motion of the second digit of the right hand. This data could be used to select a region of interest while the subject is in the scanner. In addition, each panel of the display may contain a scale 8020, and a numerical index for the scale 8030 that may include measurement units. The subject or device operator may view each planar section within the scan volume in any plane of section, showing the level of the activation map. The corresponding anatomical section may be presented as well. The subject or device operator may use a pointing device such as a mouse to indicate the position of a region of interest 8050 based upon the area(s) that show activation on one or more of the sections shown. The subject or device operator may also zoom in or out on any section to more accurately localize are area of activation.

At this point, activity metrics are computed for this selected area or volume, and results may be displayed substantially immediately. This process may take place in a limited period of time. This period of time may be within 10, 5, 2, 1, 0.5, 0.25, 0.1, 0.01 or less seconds from the time of collection of the data. This process may take place while the subject is still in the measurement apparatus, such as the scanner. This process may take place prior to testing of the subject. The timecourse of the average activity for this bounded area is computed and displayed 8100, as well as the PETH for this area triggered on the beginning of each 30 second rest period 8200. Each of these may be displayed with their corresponding timescale and magnitude scale, and may additionally include standard error or standard deviation measures, with an example shown for the PETH. The operator can then accept the selected area of the given section as the region of interest, or repeat the process until he or she is satisfied with the region of interest that has been selected.

iii. Automated Physiological Definition of Region of Interest

Regions of interest can be defined automatically using numerical criteria based upon the voxels of a scan volume, or a sub-region of a scan volume. These automatically defined regions of interest can then be presented to the subject or device operator for acceptance or alteration. This process may take place in substantially real time, and may take place while the subject is still in the measurement apparatus

Numerical criteria based upon the computed activation images/volumes can be used to determine whether individual voxels are to be included within a region of interest. In one embodiment, the process involves performing a number of physiological localization trials, and processing the resulting scan volume data into activation maps. The scan volumes may be pre-processed, and activation images/volumes may be defined. These activation images/volumes may be thresholded to select relevant voxels to be included in the region of interest. Additionally, spatial grouping may be employed, such as to reject voxels that are not adjacent to other selected voxels.

In one example, the 30 second rest, 30 second index finger movement task is used. Pre-processing uses a 1 pixel gaussian spatial filter using methods as described in Examples section 1. % BOLD difference activation volumes may be computed that correspond to: 100%×(the average computed for each voxel for all scan volumes from periods starting within 5 seconds after the start of behavior until the end of behavior, minus the average computed for each voxel for all scan volumes from periods starting within 5 seconds after the start of rest until the end of rest) divided by the average computed for each voxel for all scan volumes from periods starting within 5 seconds after the start of rest until the end of rest. This leads to a % difference map. The voxels with large values may be the voxels that are positively activated by this task, and may include the motor cortical regions that subserve this task. A region of interest may then be defined using a difference criterion such as all voxels with a difference value above a certain criterion, such as 0.5%. Voxels may be further selected by disregarding all voxels further than a criterion distance, for example one voxel, from a criterion number of other voxels above the threshold, such as one voxel.

One criterion used for automated physiological definition of a region of interest is a difference criterion, such as the average difference in % BOLD activation level between the stimulus or behavior condition and background, as just described. Another criterion used for automated physiological definition of a region of interest is a t-statistic criterion, such as a t-test statistical contrast comparing voxel values during a stimulus and a rest condition. Another criterion used for automated physiological definition of a region of interest is a statistical criterion, such as a an F-test statistical contrast comparing voxel values during a stimulus and a rest condition. Another criterion used for automated physiological definition of a region of interest is a correlation, such as the correlation of the activation of a voxel with the stimulus or behavior condition across repeated trials. Another criterion used for automated physiological definition of a region of interest is an additional statistical measure, such as the general liner model, non-parametric statistics, and corrections for repeated measures and spatial features as described in the documentation of existing MRI/fMRI/PET data processing packages. Another criterion used for automated physiological definition of a region of interest may be any of those described for the computation of activation maps or activity metrics in Examples section 1.

Once an ROI has been automatically determine, it can be analyzed just as with a described for a manually determined ROI in section ii above. The timecourse of the average activity for this bounded area may be computed and displayed, as well as the PETH for this area triggered on the beginning of each 30 second rest period. The operator may then accept the selected area, modify it by adding or removing voxels or areas, or repeat the process until he or she is satisfied with the region of interest that has been selected. This allows the user to select regions until the region that is most strongly activated by the stimulus has been determined

5. Determining a Set of Effective Stimuli or Behaviors for a Particular Subject

Once the region of interest has been identified, optionally, stimuli or behaviors may be evaluated by monitoring the physiological activity response in the region of interest in order to determine stimuli or behaviors that are effective and relatively more effective in altering the physiological activity of the region of interest.

It is important to note that stimuli or behaviors that are effective for altering the physiological activity of a given region of interest for a first subject may not also be effective for a second, different subject. Hence, the present invention contemplates that the stimuli or behaviors used to alter the physiological activity of the region of interest should be individualized for a given subject. Described herein is an evaluation of the stimuli or instructions for behavior for an individual subject in order to select the most effective stimuli or instructions for behavior for that subject. It should be noted that the step described in section 5 of selecting the most effective stimuli or instructions for behavior for that subject is optional, and may also not be carried out, instead using the effective stimulus set described in section 1.E.

Determining effective and more effective stimuli or behaviors may be performed by presenting a series of different stimuli or instructions for behavior from a set of exemplars one or more times, determining an activity measure or index for each different stimulus or behavior from one or more brain regions of interest, comparing the effect each different stimulus or behavior had, and selecting the one or more stimuli or instructions for behavior that had the most desired affect on activity. By performing this selection process, the most effective stimuli or instructions for behavior may be identified for a given region of interest for a given subject.

Described below is an example of a process that may be used to determine a set of effective stimuli or instructions for behavior.

The subject may be in an fMRI scanner as described, and physiological measurements may be conducted repeatedly throughout to measure scan volumes. A series of trials may be conducted, each trial consisting of a 30 second rest or background period, followed by a 30 second period of activation by a behavior. For each trial, first the subject is initially allowed to rest for 30 seconds. A stimulus or instruction for behavior is then selected. This selection may be a random selection. Additional selection methods are described in Examples section 3 below. The selected stimulus or instruction for behavior condition is then employed. Optionally, this includes presenting the stimulus or instruction to the subject using a subject user interface, such as a display that can be viewed by the subject. The activation for the selected stimulus or behavior may then measured as the % BOLD difference in average activity within a region of interest during the stimulus or behavior compared with during the rest period. Any of the activity metrics described in the examples may optionally be used to define the stimuli or behaviors that lead to the greatest responses or changes in the activity metrics.

This process is repeated for different stimuli or instructions for behavior until all the stimuli or instructions for behavior to be evaluated have been presented, or until stimuli or instructions for behavior have been identified that provide a desired level of activation. The stopping point can optionally be defined by a selected number of repetitions of each condition, or a variance-based measure of certainty regarding the response to each stimulus or instruction for behavior, such as the certainty of a maximum likelihood measure of the most effective stimulus or instruction for behavior.

Based upon the activity metrics observed for each stimulus or instruction for behavior, certain stimuli or instructions are selected. This selection is typically made by selecting a small number of stimuli or instructions for behavior from the complete set that elicit the largest activation in the region of interest. The more effective stimuli or instructions for behaviors are then used as the exercises for the subject.

6. Testing of a Subject and Analysis of Data

The invention disclosed may be used for testing or diagnosing subjects, such as the testing of subjects modulation of selected brain regions. Once a brain region of interest has been localized and effective stimuli or instructions for behavior have been selected based upon their ability to modulate the brain regions of interest, these stimuli or instructions for behavior may be used to test the subject. Alternatively, the subject may be tested in the absence of any overtly presented stimuli or behaviors, using a subject resting state during the testing period.

An example of the type of data that may be produced in this fashion is a map of the variance of signal computed for each voxel of the brain of the subject over some time period. Another example is the correlation of the signal computed for a voxel of the brain with one or more additional voxels, all measured over some time period with some sampling rate. In addition, measures of this type may be compared with standard values derived from normal or disease populations.

Testing may comprise performing trials comprised of alternating periods of rest, followed by exercise, or periods of different types of exercise. These trials may be designed to engage the regions of interest of the brain using the selected set of effective stimuli or instructions for behavior. These alternating periods of rest and performing a task are typically formed together into testing blocks that last at least 0.25, 0.5, 1, 5, 10, 20, 30 or more minutes, with physiological scanning beginning at the start of a testing block, and taking place during each testing block. Testing blocks may be periodically repeated, with 1-50 testing blocks taking place in one testing session, and multiple testing sessions taking place on the same day or on different days. The progress and physiology of the subject may be measured frequently during the testing block.

Data from subject testing is preferably recorded and stored. This allows the progress of the subject to be monitored. For example, a common type of information that may be relayed is an average level of the activity metric for the region of interest that the subject was able to achieve during each testing trial, testing block, and testing session. This information may also be recorded to a more permanent recording medium, such as a computer disk storage device. Any and all raw data and computed measures may be stored for later recall.

A. Conducting Trials

During testing, subjects may participate in a series of testing trials, and physiological measurements may be made repeatedly at fixed intervals throughout. Testing may also take place in the absence of physiological measurement as described in section 6.J. During a trial, the subject may first be allowed to rest for a period of time, a stimulus or behavior may be selected to activate the particular region of interest. The measurements taken during rest provide a baseline so that the effect the stimulus or behavior has can be better measured. It is noted that the rest measurement can precede or follow the measurement associated with the stimulus or behavior. Alternatively, all measurements may be made during a rest period.

As an example, a behavioral trial within an fMRI scanner may consist of the subject first resting, and then making eye movements to a series of visual targets that are presented. The trial may begin with the presentation of an instruction for the subject to rest for a period of time. The stimulus or behavior that will be used in the trial may then be selected by the analysis and control software and then presented to the subject, such as the instruction to make an eye movement to a presented target. This instruction may lead the subject to begin an exercise using any stimuli necessary to conduct the exercise. The subject may then perform the exercise, typically for a 30 second or 1 minute period of time. In testing designed to activate a different brain region, the subject might be instructed to view or imagine a particular face to activate a face-selective brain region, or engage in a sensory discrimination test to activate a sensory region. After performing the exercise, the subject is again allowed to rest. After the rest, the subject may be asked to respond to a question in some cases, such as selecting whether a stimulus presented in the trial contained a particular feature. The testing trial may then be repeated multiple times during the block.

Some aspects of this process are explained in further detail in the following sections.

B. Measuring and Displaying of Physiological Activity

Substantially throughout the process of testing, the physiology of the subject may be measured in the scanner. This information may be presented, and may also be used for additional computations such as the computation of metrics from a region of interest. This process takes place at a regular or irregular repetition rate, such as one set of measurements per second in one example, or at an alternate sampling rate.

i. Physiological Measurement

While the subject engages in testing, data are acquired and processed about the resultant brain activation. This process has been described above in sections 3.D. and 3.E. and FIG. 1. In summary, this process may comprise:
collecting raw data as described in section 3.D.i
reconstructing the result into images/volumes as described in section 3.D.ii.
pre-processing the result as described in section 3.D.iii.
computing activation images/volumes from the result as described in section 3.D.iv.
computation of activity metrics from the result for defined region(s) of interest as described in section 3.D.v.

ii. Displaying Physiological Activation Maps

Many varieties of measurements may be made, and resultant computations performed and results displayed. Once activation images/volumes and activity metrics have been computed, they may be displayed to the device operator, or to remote parties. This display can include physiological images of the subject's brain, matched anatomical images at the same level of section, 3-D reconstructions of either anatomy or physiological activation patterns, and both difference activity level images and statistical maps, maps of signal variance, or maps of level of correlation in activity between voxels. This section describes one example of information displayed. Further detailed examples of displays are described in examples sections 1 and 2.

In one example, the T2* weighted activation is measured in a 64×64×17 voxel scan volume corresponding to a 22×22×12 cm volume of a subject's brain. The subject engages in testing involving a repeated task of 30s rest and then 30s imagined finger motion. Data are converted into scan volumes once per second in a process requiring less than one second. In this example, no pre-processing is used of the scan volumes generated. Scan volumes may be turned into variance maps by computing the variance of activity for each voxel over the time period of measurement.

Viewing this activation map may allow the device operator or other individual to assess the activity in the brain region of interest.

iii. Displaying Activity Metrics

From the variance map, other activity metrics may be computed corresponding to the physiological activity in a region of interest. A first activity metric may be the average variance in the selected region of interest, for example an area including the primary motor cortex. This display may take the form of a line chart.

Activity metrics may also be measured for comparing regions of interest. It may be useful to measure activity metrics for comparison regions of interest to serve as a negative control for the primary region of interest, indicating that testing has a selective effect on the primary region of interest rather than on broader areas of the brain. The activity seen in these metrics are frequently an indication of the overall arousal state of the subject. Information is also computed about the difference in activation between the primary region of interest and a secondary region of interest, which provides a selective measure of the change within the region of interest less any overall changes affecting the brain more broadly.

iv. Displaying Movement Metrics

Another type of metric typically computed during testing may be a set of movement metrics. The data collected may be used to derive information on the position of the subject within the scanner, and this in turn may be used to determine an ongoing measure of the subjects translational movement in 3-D, as well as roll, pitch, and yaw. Movement metrics allow assessment of the movement of the subject. Movement information may also be fed into computations that allow for movement correction of the scan volumes collected. Examples of the computation of movement metrics is described in Examples section 1.D.v.

C. Influencing Subject Behavior

As has been noted previously, a feature of the present invention is the performance of exercises where information, stimuli or instructions for behavior are communicated to the subject through visual, auditory or other signaling.

i. Selecting the Next Stimulus/Behavior

A stimulus or instruction may be given to a subject representing something to perceive, or a suggestion for what the subject should do, such as to engage in an action or cognitive activity. The software may select what stimulus or behavior the subject will be engaged with for a trial. When the subject begins to perceive this stimulus, or engage in this behavior, this will cause a set of related changes in the brain of the subject. These changes may also be measured. In some cases, the subject may provide an overt response to the selected stimuli or instructions as well, as would be the case if the subject were completing a sensory discrimination task.

The stimulus or behavior used in a trial may be selected from the effective stimuli or instructions for behavior set. This selection may be a random selection from the effective stimuli or instructions for behavior set, may be based upon performance, or may be guided by the subject themselves or by the device operator. For the purpose of testing a subject, the object of a trial may be to maximally activate one or more discretely localized brain regions.

ii. Displaying Stimulus to Subject

A stimulus may be presented to the subject for the subject to experience. Visual stimuli may be presented on one of the display panels viewed by the subject or the device operator, for example as described in FIG. 4, or other display elements as described in section 3 or in the examples. Stimuli may also be presented to subjects using additional stimulation devices providing for stimulation other than visual stimulation, such as using auditory, tactile, proprioceptive, odorant, temperature, gustatory or other stimuli.

D. Analysis of Subject'S Brain Activation

Once physiological data has been collected, the subject's modulation of the activity metric(s) can be assessed. A number of measures can be computed of the subject's brain activity.

i. Activation Performance for a Trial

The subject's activation performance may be monitored throughout each trial. The activity metric that is monitored may include one or more activity metric being measured from a region of interest. Typically the activity metric may compare an activity level metric between a rest period and an exercise period of a trial such as the period when the subject is engaging in a task, perceiving a stimulus, or attempting to modulate the level of an activity metric. Alternatively, the activity metric can be measured only a rest period, without comparison to a task period. One type of activation performance measure may be the difference between the average of the activity metric during the stimulus/behavior period and during the rest period. Another type of activation performance measure may be the average of the activity metric during the stimulus/behavior period alone. Another type of activation performance measure may be the average of the activity metric during the rest period alone.

ii. Activation Performance for Multiple Trials

Once activity metrics and trial success computations have been computed for individual trials, they then may be combined to analyze the subject's performance across trials. For instance, the percent of behaviorally successful trials may be computed as the percent of trials when a subject successfully performed an instructed behavior. The percent of correct trials may be computed and displayed for different trial types or periods of time.

The level of difference in activation between the stimulus/behavior condition and the background condition may also be averaged for multiple trials, or computed and displayed for different trial types or periods of time.

E. Analysis of Subject'S Behavioral Performance

If subjects are performing a behavioral task and therefore making overt behavioral responses during the trial period, then their performance at this task is analyzed to assess their behavioral performance. For instance, if a subject is performing a visual stimulus discrimination task designed to activate visual sensory areas during testing, then performance on this task may be computed for each trial. For each trial, the subject provides a response (e.g. a button-press indicating which of two alternative areas contained a visual stimulus). The analysis and control software records these responses and makes computations of the subjects performance level. These computations correspond to typically measured psychophyisical parameters (see Green, D. M. and Swets, J. A. Signal detection theory and psychophysics. New York: Wiley, 1966). For instance, if sensory discrimination is being made on a number of stimuli along a continuum from easy to hard, the percent correct for each stimulus type is computed in order to generate a performance curve and determine a 50% correct threshold. Percent correct measures may be made in the same fashion for motor or cognitive tasks. These allow the computation of psychophysical parameters such as d′ and beta according to standard methods familiar to one skilled in the art. The subject may be informed on each trial whether their response was correct or incorrect.

F. Repeating Trials and Testing Blocks

Testing trials as described thus far in section 6 may be repeated throughout a block, typically lasting 1-60 minutes with substantially continuous physiological measurement throughout. Testing blocks then may be repeated as well, with 1-50 blocks taking place in one session, and multiple sessions taking place on the same day or different days.

G. Recording Progress of Exercise and Treatment

The subject's activity metric over each testing session is monitored. A principle type of information may be the average level of the activity metric for the region of interest for the subject during each trial, block, and session. It should not be lost that testing may take place during concurrent therapy for the subject such as pharmacological or behavioral therapy, or changes in the subjects condition. Accordingly, it is important that the progress of the subject also be measured in terms of signs and symptoms of the condition being treated, as well as behavioral performance. This information may also be presented to the device operator or another person involved with the process, such as the subjects physician. This information may also be transmitted to a remote site, such as via the interne or other communication media.

H. Prescribing Ongoing or Follow-On Testing as Needed

The testing described in this invention can be combined with additional forms of intervention, such as pharmaceutical treatment, nervous system stimulation, or rehabilitative medicine treatment. Accordingly, a medical professional monitoring the progress of the subject in regard to the subject's condition may prescribe additional therapy or alter therapy as the need arises. Information for this decision process may be derived from the subject's activity metrics.

7. Comparison of Subject Data with Group Database

The activity maps or activity metrics that have been measured for a subject may be compared with values measured previously from groups of subjects. In this way, the values of the subject may be used to create indicators of the activity in the subject's brain as compared with standard values or values in populations with various conditions.

A. Maps of Percent of Normal Values

An example of comparing a subject's data with normal values is the computation of a “percent of normal map”. A percent of normal map expresses the value of an activity metric computed for the subject at each voxel as a percentage of the mean or median value for the activity metric previously measured from a population of individuals assumed to be normal, or standard with respect to this activity metric.

B. Comparison of Activity Metrics for a Subject with Groups

An example of comparing a subject's data for an activity metric with a group is to compare the measured variance of signal from within a region of interest in the subject with the distribution of measures of this activity metric previously measured from a population of subjects. In this way, the rank of the subject's level within the population can be assessed. An example of this rank would be the determination that a subject's variance was in the 2nd quintile of values measured for the group, or that the subject's variance was at the 23rd percentile of the group. This allows the level of an activity metric measured for a subject to be compared against a known reference. Through making this comparison, it is possible to determine whether the subject's measured values are higher, lower, or near the norm for the known reference group. This process may take place for a single region of interest, or for the entire brain of the subject voxel-by-voxel to produce a distribution of values. This may take place after the voxels of the subject have been spatially registered to the standard group.

8. Evaluation of Subject Data

A. Diagnosis of Subject Condition

For an activity metric that has been measured for a subject, the value of that metric can be compared with the distribution of two populations of subjects measured previously in order to determine the probability that the subject is in one vs. the other of those two populations. For example, if the variance of signal strength from a in a frontal cortical region associated with depression has been measured from a subject, and the value of this activity metric has been measured previously from a population of patients with major depression and a second normal control population, then these values can be compared. For each level of the activity metric (variance of the frontal region of interest), some fraction of the depressed patients and some fraction of the normal controls would have this value of the activity metric. If the subject being studied has this level, then the a probability estimate can be created that the subject is in the depressed group which is, for example, the ratio of the likelihood of a patient in the depressed group having this value of the activity metric, divided by the combined likelihood of subjects from either depressed or normal groups having the measured value of the activity metric. This is one means of calculating a probability estimate of the subject being within a population having a particular condition, in this case depression. Multiple estimates may be calculated for a subject in this fashion using multiple activity metrics. These can either be viewed independently, or combined to produce an overall estimate of likelihood that the subject is in a group with a particular condition. In this way, the subject may be diagnosed with regard to their likelihood of having a given condition. This method also produces a reliability measure of the diagnosis given based upon the statistics of false-positive using the measured distributions, based upon standard principles of diagnosis, statistical distributions and epidemiology. One skilled in this art will be familiar with alternative procedures for diagnosing the likelihood that a subject is in a group with a given condition. A second example of diagnosis of a subject's condition would be to use the observed correlation between selected brain voxels in the subject, and compare these measured values with the distribution of values from a population of interest, such as patients with a neurological injury in this region vs. normal controls. In this way, following the same logic as above but using the activity metric of correlation of a voxel's activity with that of other selected voxels, the likelihood of neurological injury to the selected area can be estimated. This estimate can alternatively be conceived of as an index of tissue health based upon the correlation of activity of the tissue with other tissue areas.

B. Selection of Metrics for Comparison

In order to select which activity metric for a subject should be used for comparison to diagnose the likelihood of presence of a given condition, a metric should be selected which strongly differentiates a group of previously-measured subjects with that condition from a second group of previously-measured subjects who do not have that condition. This means that the distribution of values of the metric for the group of subjects with the condition should have as little overlap as possible with the distribution of values for the group of subjects without the condition.

9. Addition of Subject Data to Group Database

Once the data for a subject have been analyzed, they may be stored electronically. Through the repeated application of this process, a database of values may be generated for many activity pattern metrics for each subject. An example of information that may be stored in a subject database is descriptive information for each subject, such as medical treatments, medical history, psychological or psychiatric measures, performance abilities such as test scores, and so on. Another example of information that may be stored in a subject database is anatomical data for the subject, such as anatomical brain scans. Another example of information that may be stored in a subject database is functional brain maps for the subjects. Another example of information that may be stored in a subject database is lists of values of activity metrics from brain regions of interest. Another example of information that may be stored in a subject database is variance measures from each voxel in the brain of the subjects. Another example of information that may be stored in a subject database is correlation measures between voxels in the brains of the subjects.

### EXAMPLES

The brain is highly segmented, with localized regions of the brain performing entirely different functions. Now that such selective testing of regions of interest of the brain can be achieved, a myriad of valuable applications are made possible. Described herein is a non-comprehensive list of different applications of the methods of the present invention. Also described are more detailed examples of the types of information that may be provided to subjects and of the types of computations used to generate these displays.

1. Performing Computations on Images Using Analysis and Control Software

The data analysis/behavioral control software 130 may be used to take in raw image data and perform a series of computations, including pre-processing 135, computation of activation image/volumes 137, computation of activity metrics 140. A single example of these steps were presented in sections 3-6 above. The following sections provide more detailed examples and explanations. The results of the computations described here are presented to the subject of the experiment or used to control its progress. It is noted that the examples provided herein relate to fMRI data processing. However, analogous methods may also be developed for other types of physiological data. The examples presented here can be performed using the functions developed in Matlab version 6.1 provided by the Mathworks, Inc., and its associated toolboxes such as the statistics, image processing, and digital signal processing toolboxes.

A. Data Pre-Processing

Physiological data received by the analysis and control software are in the form of raw T2* weighted 2-D or 3-D scan images/volumes 125. These data can be pre-processed using a variety of methods. One type of pre-processing that may be performed on the input image/volume data may be to pass the input image/volume data as output through to the next step of computing activation images/volumes without any further pre-processing. The resultant output is a set of 2-D or 3-D scan images/volumes that have undergone computations as described. Each of the methods described in this section can take the raw image/volume data 125 as its input, or can take the output of one of the other methods described in this section as its input. Further detail on each of these methods is provided in user manuals for Matlab ver 6.1, as well as in the user manuals and documentation for existing MRI/fMRI/PET data processing packages.

i. Spatial Smoothing

One type of pre-processing that may be performed on the input image/volume data may be spatial smoothing according to standard methods to produce smoothed image/volume output data. This is useful because it removes noise in the data, improves statistical properties by making the data variance more gaussian, and produces an image that is easier to interpret visually. This is accomplished by convolving the data with a 2-D or 3-D gaussian filter function with a defined half-width.

ii. Temporal Filtering

Another type of pre-processing that may be performed on the input image/volume data may be temporal filtering including lowpass, highpass, bandpass filtering and convolving with a function such as a hemodynamic response function. This is useful because it removes temporal noise in the data, matches the signal power in the data to that corresponding to the trials being conducted, and improves later data processing and statistical measures. This is accomplished by convolving the data with a temporal filter. This convolution will normally be with a causal filter as the data is being collected in substantially real time. The filter can be a highpass filter, such as a highpass filter with the cutoff of 10, 30, 60, 120, 240, 300 s, or the lowest relevant frequency component of the behavioral trials being conducted, or a drift rate that reflects the slowest relevant physiological change expected in the signal. The filter can be a lowpass filter, such as a lowpass filter or gaussian function with the cutoff of 0.25, 0.5, 1, 2, 4, 5, 10 s. The filter can be a lowpass filter designed to match the shape of a hemodynamic response function modeled as an alpha function. The filter can be a bandpass filter that accommodates a combination of highpass and lowpass characteristics. These filters can be designed using standard digital filter design techniques.

iii. Slice Time Correction

Another type of pre-processing that may be performed on the input image/volume data may be slice time correction to correct for the time of collection of each slice by interpolation. This is useful because it approximates the case where each slice in a scan volume was collected simultaneously. In order to perform this computation, the relative times of collection for each slice in a scan volume are known. The first image in each volume is taken as the reference image. The output values for each successive image in the volume are computed as the interpolated value between the measured value for each voxel in the image and the measured value for the same voxel in the previous image or succeeding. The interpolation yields the value corresponding to the estimated value for the voxel at the time point actually measured for the reference image. This standard method is described in the literature and in manuals for existing MRI/fMRI/PET data processing packages.

iv. Transformation into Standard Coordinates

Another type of pre-processing that may be performed on the input image/volume data may be a transformation into standard coordinates by applying a transformation vector that yields the corresponding value at each voxel in a standard coordinate space. This matrix is predetermined as described in Examples section 6. This has the advantage that all subsequent processing and display of data is in a standard coordinate space such as Talairach space or MNI space that can be directly compared with reference data.

v. Resampling of Data

Another type of pre-processing that may be performed on the input image/volume data may be resampling to increase or decrease the temporal and spatial resolution of the data, using band-limited filtering if needed. Resampling can produce a more detailed or less detailed view of the collected data. It can also be used to match the sampling of the data to that used in data set to which it will be compared, such as anatomical data collected for the subject, or data from a standard subject. Resampling can be performed using standard methods.

vi. Motion Correction of Data

Another type of pre-processing that may be performed on the input image/volume data may be motion correction to adjust for the motion that takes place between subsequent scans. This is useful because each section of each volume is in substantially the same position as in the first or reference scan of a scanning session. This can take place by applying using a transform created for each scan volume to that scan volume. The transform is designed to create the best fit in the least-squared error sense between the data of the current scan and the reference scan, including translation, rotation, and scaling if needed. An example of this software is described in: CC Lee, et al. Real-time adaptive motion correction in functional MRI. Magn Reson Med 1996;36:536-444 and in manuals and literature associated with existing MRI/fMRI/PET data processing packages. Each of these steps, which can take place individually or in combination and in any order, will be familiar to one skilled in the art. These pre-processing steps may be applied to one or more reference scan, typically an early scan from the scanning session that will be used as a basis of comparison for computing activation images/volumes. These pre-processing steps may also be applied to each successive scan collected. The pre-processing for the reference scan(s) need not be the same as for subsequent scans. These pre-processing steps lead to pre-processed scan volumes for each sampled time point, which are then used for further computation and processing. The use of motion correction software may be used to allow motion of the subject relative to the measurement apparatus while measurements are collected and/or testing is conducted, those measurements being corrected so that voxels correspond to the appropriate locations within the brain of the subject.

viii. Regression Filtering

Another type of pre-processing that may be performed on the input image/volume data may be regression filtering to remove noise components associated with exogenous events. For example, the activity level in each voxel may be correlated with an event not directly related to testing, such as the phase of the cardiac or respiratory cycle, or movement of the subject brain. The data from each voxel may be corrected by regressing out this noise source. This method is described in the literature, for example in J. T. Voyvodic, Neurolmage 10, 91-106 (1999).

viii Selection of Voxels Corresponding to Brain

Another type of pre-processing that may be performed on the input image/volume data may be the selection of voxels corresponding to the brain. This process may include the masking off of voxels determined to be outside of the region corresponding to the brain, such as voxels corresponding to the skull and regions outside of the head. This process may also include the masking on of voxels determined to be inside the region corresponding to the brain. This process may take place automatically under software control. Algorithms for this process are described in the literature and is known to one skilled in the art.

B. Computation of Activation Images/Volumes

Activation image/volumes may be computed taking as input a set of the pre-processed scan images/volumes, normally the entire set generated since a scanning session began. The activation image/volumes that are generated as output indicate the level of physiological activation at each voxel on the map. These maps may represent various measures of the second-by-second blood oxygenation level in the subject's brain regions that is an indicator of blood flow, and of brain metabolism and neural activation. These activation images/volumes, in turn, may be used as input to generate additional activation images/volumes, or to compute activity metrics from localized brain regions. These activation images/volumes may also be used as inputs to the displays that will be presented.

i. Raw T1, T2, or T2* Weighted MRI Signal

One type of activation image/volume that may be computed is the raw T1, T2, or T2* weighted MRI. This is the pre-processed output from the previous step. In this case, no further processing is performed at this step. This is useful primarily as a display of the raw signal, for example to appreciate any potential problems with data acquisition.

ii. Difference Images Including BOLD Difference Images/Volumes

Another type of activation image/volume that may be computed is the difference image, including BOLD difference images. One primary type of difference image is the measured difference in level between two time points. A single T2* weighted image by itself gives little information about the activity level at each voxel position, because the values measured primarily reflect the anatomical composition of the underlying tissue with a small contribution (e.g. 1%) from the physiological signal. By comparing images measured during different conditions, the anatomical portion of the signal will be essentially unchanged, but the portion of the signal corresponding to the physiological activation will be different. This is useful because it provides a measure of the change in physiological activation between two time points. Thus, the difference in T2* signal intensity between two time points is an indicator of the difference in physiological activation between those two time points. There are a variety of choices of what difference to compute, for example how many time points to average over before computing a difference.

Normally, a reference scan image or volume may be selected, which may then be subtracted from subsequent images or volumes. This reference volume can be the first scan of a session, or one of the early scans of a session because the first scan may be unrepresentative due to tissue magnetization not having reached steady-state.

One difference image/volume can be computed by subtracting the value at each voxel in the reference scan from the value in the currently measured scan. Another difference image/volume can be computed by subtracting the average value over a defined time period before the current scan from the value in the currently measured scan, useful if the steady-state level measured is drifting over time. Another difference image/volume can be computed by subtracting the time-filtered and/or spatially smoothed value from a time period before the current scan from the value of the currently measured scan, also useful to reduce noise and correct for baseline drift. Another difference image/volume can be computed by subtracting the average value from a series of reference scans collected during one or more background or rest conditions, useful when an average background level is the most appropriate for taking a difference. Another difference image/volume can be computed by subtracting the average value from a series of reference scans collected during one or more behavior or stimulus conditions, useful when an average activated level is the most appropriate for taking a difference.

iii. % Difference Images/Volumes

Another type of activation image/volume that may be computed is the percent difference image/volume, computed by normalizing the measured difference image/volume in order to produce an image/volume in units of fractional difference, or percent difference. For example, a % BOLD difference image/volume is computed by taking a single difference image/volume and dividing it by a reference image/volume. At each voxel, the resultant % BOLD signal equals, for example 100%×(signal at time point−signal at reference time point)/(signal at reference time point). % difference signal images/volumes can be computed by taking any of the above difference signal images/volumes, and dividing them by their corresponding reference or average reference images/volumes.

iv. Variance Images/Volumes

Another type of activation image/volume that may be computed is a variance image/volume. The variance of any pixel or group of pixels over a period of time can be computed, and these values can be formed into a variance image/volume. These images can be useful in located blood vessels, which might be excluded from further analysis in certain instances where brain matter physiology is the target, or focused upon if vascular perfusion is the target. Variance can also be expressed as standard deviation, or coefficient of variation of activation measured from each voxel, using methods familiar to one skilled in the art.

v. Statistical Contrast Images/Volumes

Another type of activation image/volume that may be computed is a statistical contrast image/volume. Images and volumes can also be computed based upon statistical measures of activation for each voxel. This may be useful because these maps indicate measures of the reliability with which a given voxel's activity correlates with some condition(s), such as a stimulus, or behavior. One type of statistical contrast map that can be computed may be a t-test map, that may compute the p-value from a t-test comparing the set of measurements for a voxel during one condition, such as a background or rest condition, with the measurements during a different condition, such as a stimulus or behavior condition. Another type of statistical contrast map may be an F-test map, that may make a comparison of these same sets of measurements using an F-test and a predictor model such as a boxcar or sin-wave function representing different behavioral periods, or a boxcar function convolved with a haemodynamic response function such as an alpha function. Another type of statistical contrast map is a map that may be corrected for the large number of degrees of freedom inherent in fMRI data reflecting serial measurements, or corrected for spatial correlation among proximate voxels. The computations involved have been described extensively in the literature, and in the manuals and supporting literature for existing MRI/fMRI/PET data processing packages.

vi. Contour Maps of Activation Images/Volumes

Another type of activation image/volume that may be computed is a contour map, which may be computed to designate the contour lines on an activation image or volume for a set of one or more contrast levels. This may be useful for displaying and viewing activation images/volumes, or for localizing regions of activation.

vii. Thresholded Maps of Activation Images/Volumes

Another type of activation image/volume that may be computed is a thresholded map. Thresholds may be computed and used to cut out certain most relevant portions of the data from activation images/maps. Thresholds can be defined as a mean value of a region, or some fraction of the mean value. The fraction can be defined by a measure of the variance. An example threshold would be two standard deviations below the mean value of an entire activity pattern image. In some cases it may be helpful to set all values below or above a set threshold to a background level.

C. Displaying Activation Images/Volumes

Anatomical and physiological data representations may be presented using a display or printed out. In addition, these data may be presented to a device operator on one or more additional displays. In one embodiment, activation image/volume data from an fMRI is transformed into a variety of intensity-coded or color-coded 2-D image maps. These maps may be presented a 2-D sections, such as coronal, sagittal, axial, or oblique sections. They may also be presented as 3-D images such as transpart or cutaway volume images, rendered 3-D volume images, or wire-mesh images. Physiological measurements can also be overlayed onto anatomical measurements either using 2-D anatomical images or 3-D rendered brain images. These methods are familiar to one skilled in the art and are described in available documentation for existing MRI/fMRI/PET data processing packages (see definitions). The resultant images are presented using the displays described in Examples section 2.

D. Computation of Activity Metrics

Data from activation images/volumes can be used to compute activity metrics. These activity metrics are computed measures from regions of interest within activation images/volumes. The input to these computations are the time series data from a single measurement point or voxel, or from a group of voxels that constitute a region of interest or an entire image or volume. A simple example of an activity metric is an average value at a single time point for all of the voxels within a region of interest. Some example activity metrics are described here. All of these metrics may be computed in substantially real time in certain preferred embodiments.

i. Average Value Metrics at a Single Time Point

One type of activity metric that may be computed is the average value from a region of interest at a single time point. This value gives an indication of the average level of activation for the region of interest.

ii. Spatial Pattern Comparison Metrics

Another type of activity metric that may be computed is a spatial pattern comparison metric. Spatial pattern comparison metrics can be used to compare the pattern of activity in a region of interest with a target or reference pattern. This is useful, for instance, if a subject is being tested to approximate a target pattern of activation. One type of spatial pattern comparison metric can be computed as the sum of the voxel-by-voxel differences between the current pattern and the target pattern in an ROI, indicating overall closeness to the target. Another type of spatial pattern comparison metric can be computed as the sum of the voxel-by-voxel sums of the current pattern and the target pattern in an ROI. The two preceding spatial pattern comparison metrics can be divided by the target pattern sum to give a percentage value. Another type of spatial pattern comparison metric can be computed as the dot product between the vector comprising the current pattern and the vector comprising the target pattern in an ROI, indicating overall closeness to the target.

iii. Correlation Metrics

Another type of activity metric that may be computed is a correlation metric. Correlation metrics can be computed that correspond to the correlation between the activity of two voxels, or two regions of interest over time. This may be useful in testing the activity and connectivity of brain regions. One type of correlation metric can be computed as a correlation coefficient between two activity metrics, r. Another type of correlation metric can be computed as an activity-triggered average between two activity metrics, such as the average level of activity at one point for one or more ranges of activity level at another point. Another type of correlation metric can be computed using ‘network analysis’ to determine functional connectivity between different points within the brain as described in “Functional neuroimaging: network analysis”, L Nyberg and A. R. McIntosh, in HandBook of Functional Neuroimaging of Cognition eds Roberto Cabeza and Alan Kingston.

iv. Threshold Crossing Metrics

Another type of activity metric that may be computed is a threshold crossing metric. Threshold crossing information can be used to measure when an already-computed activity metric crosses a given threshold level. Another type of threshold crossing metric can be computed as an indicator of whether the signal is above or below that threshold value. Another type of threshold crossing metric can be computed as an indicator of whether there has been a change in whether the signal is above or below that threshold since the last time point, and the direction of the threshold crossing. Another type of threshold crossing metric can be computed as a positive value at time points when the threshold is crossed, and a zero value at other time points.

v. Movement Metrics

Another type of activity metric that may be computed is a movement metric. Movement information can be used to measure determine whether a subject's movement in the scanner is confounding other measurements. Movement measurements give an indication of the position or change in position of the subject's head, brain or some other anatomically defined structure within the scanner. One type of movement metric take the form of x, y, z cartesian coordinate information, as well as pitch, roll and yaw rotational information. Another type of movement metric take the form of the chance in x, y, z cartesian coordinate information, as well as pitch, roll and yaw rotational information between two time points. A position metric can be computed by thresholding the brain scan volume data to zero for values below ⅛th of the mean value, and 1 for values above this threshold, and then computing the x, y, and z values for the centroid of the resultant volume. This centroid vector can be compared with a centroid vector at a reference time such as the first scan to give measures of change in position. Subjects can be instructed to remain more still if movement exceeds certain limits. More detailed methods for computing movement metrics will be familiar to one of ordinary skill and are described in available documentation for existing MRI/fMRI/PET data processing packages.

vi. Movement Correlation Metrics

Another type of activity metric that may be computed is a movement correlation metric. Once movement metrics and activity metrics have each been computed, then metrics of the correlation between the two can be derived. These metrics are helpful in determining whether a subject's movement is contributing significantly to the activity metrics that have been observed. An F-test can be used to compute the relationship between an activity metric and a movement metric. Once a relationship has been determined, the contribution of the movement can be regressed out of the activity pattern data. This can yield measures of activity pattern data in the absence of the contribution of movement.

vii. Signal Processing Metrics

Another type of activity metric that may be computed is a signal processing metric. A number of other mathematical measures can be made on activity metrics that provide additional useful information to characterize these signals, and in turn to control them. Certain of these metrics may correspond with particular behavioral or cognitive states, and thereby be used as a measure of the presence of those states, or to test subjects in reproducing those states. For example, active states may have more power at high frequencies of an activation metric, whereas passive or relaxed states may have less power at those high frequencies. Example signal processing measures include: the power spectrum of the activity metric, the power of an activity metric within a limited band-pass filter band, and the spectrogram of the activity metric.

viii. Activity Position

Another type of activity metric that may be computed is an activity position metric, that may compute the position of highest activity within a region of interest. In this example, the voxel or group of voxels showing the highest level of an activity metric are determined. This activity position can in turn be used as a method for decoding what is being represented by mapped neural activity. It has long been known that activity in many brain areas is ‘mapped’. Activation in different regions corresponds with particular stimulus or movement features. For this reason, a center of activation at any one point on a map can be used to determine the corresponding feature on a known map as the feature that is being encoded. This may be useful in forming an estimate of what is being represented in the brain of the subject at any point or period in time. This, in turn, can be used to guide testing, such as by selecting a next stimulus of a character that is related to that which is being coded at a particular moment.

ix. Vector Average Metrics

Another type of activity metric that may be computed is a vector average metric. Vector average metrics may involve computing an estimate of the decoded object or feature being represented by a given activity pattern. One example of this decoding is the measurement of a vector average of activity. In this example, the measure of an activity metric at each voxel within a region of interest is computed, and is multiplied by a feature vector assigned to that voxel that corresponds to the voxel's underlying feature selectivity or representational function. The vectors are then averaged to produce a vector average activity metric. This vector average can be used to compute an estimated feature being represented by the underlying physiology in the region of interest. The feature vectors that area used for each voxel may correspond to what the voxel has been determined to be involved in the processing of, or to the voxel's relative position on a defined representational map such as a cortical map of visual or motor space.

For example, for visual brain areas, the feature vector for each voxel may correspond to a position in visual space, or to a combination of other visual features, that are represented by activity in the brain of the corresponding voxel. The feature vector may also be determined by a voxel's position on a visuotopic map. For auditory brain areas, the feature vector for each voxel may by the preferred sound frequency for that voxel, or to its relative position on a tonotopic map. For somatosensory areas, the vectors may be positions on the body that the voxels are involved in receiving input from, or the voxels relative position on a somatotopic map. For motor areas, the feature vectors for each voxel may be points in space reached by a motion preferentially activating the voxel involved, or may be muscle groups that are preferentially activated in conjunction with the activation of the measured voxel. They may also be the information or function designation on a motor map of the area. Taking the motor example, it has been shown that by taking the vector average of the level of activity times the preferred movement target for each of a number of points in the motor cortex, an estimate can be made of the movement target for a particular activation pattern (see Motor area activity during mental rotation studied by time-resolved single-trial fMRI. W. Richter R. Somorjai R. Summers M. Jarmasz R. S. Menon J. S. Gati A. P. Georgopoulos C. Tegeler K. Ugurbil S. G. Kim; J Cogn Neurosci. March, 2000; 12(2):310-20, Primate motor cortex and free arm movements to visual targets in three-dimensional space. II. Coding of the direction of movement by a neuronal population. A. P. Georgopoulos R. E. Kenner A. B. Schwartz J. Neurosci. August, 1988; 8(8):2928-37). In this way, the vector average method may provide one indication of what is being represented by a given pattern of activation within a region of interest.

x. Feature Decoding Metrics

Another type of activity metric that may be computed is a feature decoding metric. Additional methods are available for decoding what is being represented by brain areas through computations involving the vector of activity at a large number of points in the brain. These additional decoding metrics may also be useful in forming an estimate of what is being represented in the brain of the subject at any point or period in time. This decoding indicates that a relation is formed between different states or patterns of activity in a region of interest and objects or movements that may be encoded. Many types of methods have been developed for creating this relation (see for instance Real-time control of a robot arm using simultaneously recorded neurons in the motor cortex, J. K. Chapin K. A. Moxon R. S. Markowitz M. A. Nicolelis, Nat Neurosci. July, 1999; 2(7):664-70), and these methods may be used by this invention. Once an estimate is available of what is being represented in the region of interest, this, in turn, may be used to guide testing, such as by selecting a next stimulus of a character that is related to that which is being represented at a particular moment, or a behavior based upon what is being represented.

x. Time Average Metrics

Another type of activity metric that may be computed is a time average metric. Once the activity metrics described have been computed, they can each be averaged over periods of time. Average values can be usefully employed to compare different conditions. In one example of a time average metric, the average of an activation metric can be computed for all time points within a recent period of time to determine a subject's recent level of activation in an ROI. In another example of a time average metric, the rolling average of an activation metric can also be computed. In another example of a time average metric, averages can be computed for different types of conditions, such as the average of a metric for all time points falling within a particular behavioral or stimulation condition. In another example of a time average metric, averages can be computed for all time points falling within a background or rest condition.

xi. PETH Metrics

Another type of activity metric that may be computed is a pen-event time histogram metrics (PETH) metric. PETH metrics are particularly useful for determining the average time course of a metric following a behavioral event, stimulus, or other event. PETH metrics are computed as the average over several trials of an activity metric, computed separately for a number of time points before or after a reference time point, such as the beginning of a trial.

xii. Likelihood of Behavioral Success Metrics

Another type of activity metric that may be computed is a likelihood of behavioral success metric. There are some time periods when a subject is more likely to succeed at a given task than others. It is generally desirable to identify when a subject is most likely to succeed or have a positive outcome in performing a behavioral task such as a perceptual or behavioral task or testing. For example, when the occipital or temporal cortical brain regions subserving the visual perception of a particular stimulus are activated, and frontal regions involved in extraneous tasks such as unrelated thoughts are not activation, the subject is more likely to succeed at a visual discrimination task. Related findings have also shown that people remember better when areas of the brain involved in memory are more active. Previous studies have documented this retrospectively. Prospective measures of a subject's activity in a region of interest involved in subserving a given task can be used to predict when the subject will have a positive successful behavior, or perform a task quickly, or learn or remember more effectively. Therefore, these measures are helpful in testing and exercising the subject.

A measure of the likelihood of success in any task can be made based upon an activity metric measured before or during a task if there is some correlation between the activity metric and success in the task. A relationship may be measured between the distribution of activity metrics over many trials, and the distribution of success at performing a task over many trials. This relationship may include an average likelihood of behavioral success for each of a number of ranges of the distribution of the activity metrics. Using this relationship, it may be possible to form an estimate of the likelihood of behavioral success for a trial conducted when the activity metric is at any particular value.

Take for example, an activity metric that varies primarily over the range of 0-1%, and 100 observed trials of a behavioral task that the subject gets right on 50% of occasions on average. The average percent correct trials can be computed for all of the measured trials that followed a 5 second period when the measured activity metric was between 0.2 and 0.3%. Similarly, the average percent correct can be computed for all other 9 increments from 0-1% for the activity metric. If there is a correlation between the activity metric value and behavioral performance, this may lead to a curve showing that at the low values of the activity metric, the subject got less trials correct on average, whereas at the high values, the subject got more trials correct on average.

Likelihood of success metrics can be computed separately for different stimuli or behaviors. For example, one observed pattern of activity may correlate with a high likelihood of success for one stimulus or task, while a different pattern correlates with a high likelihood of success for a different stimulus or task. Computing the likelihood of success for both stimuli/tasks allows the selection of whichever stimulus or task is more likely to be successful at a given moment.

Using the relation between the activity metrics and percent of positive behavioral outcomes determined by the curve, which can often be fit with a line, exponential, or logistical function, it may be possible to predict the likelihood of success on a given trial using a given stimulus from the value of an activity metric.

xiii. Multiple Regression Metrics

Another type of activity metric that may be computed is a vector of linear regression coefficients between the values of many voxels measured for a subject, and a single target voxel, with data taken over a period of collection time. The values and significance levels of the entire regression, as well as the individual regression coefficients, may provide information regarding the connectivity between brain regions. Alternatively, the values and significance levels of the entire regression, as well as the individual regression coefficients, may provide information regarding the activity of brain regions. This is because healthy, active brain tissue tends to be activated in temporal correlation with other areas of brain tissue. Therefore, a multiple regression procedure is capable of determining both the functional or effective connectivity between different brain areas, and also may provide an index of the activity or health of the tissue being measured. Another type of activity metric that may be computed is a vector of non-linear regression coefficients between the values of many voxels measured for a subject, and a single target voxel, with data taken over a period of collection time, computed using non-linear data fitting models such as neural network models, pattern recognition algorithms, or fuzzy logic models. Another type of activity metric that may be computed is a vector of linear regression coefficients between the values of many voxels measured for a subject, and many target voxels, with data taken over a period of collection time, computed using multiple linear regression. Another type of activity metric that may be computed is a vector of non-linear regression coefficients between the values of many voxels measured for a subject, and a many target voxels, with data taken over a period of collection time, computed using non-linear data fitting models such as neural network models, pattern recognition algorithms, or fuzzy logic models.

xiv. Functional Connectivity Metrics

Another type of activity metric that may be computed is a functional connectivity metric, such as the correlation coefficient between the successive values measured from one or more voxels or regions measured for a subject, and a single target voxel or region, with data taken over a period of collection time. The values and significance levels may be used as functional connectivity metrics.

xv. Combinations and Comparisons of Activity Metrics from the Same or Different ROIs

Another type of activity metric that may be computed are combinations and comparisons of activity metrics from the same or different ROIs. It is often useful to make comparisons between different activity metrics, or to compare the same activity metric for different time points, or time periods. All of the activity metrics described above can serve as inputs to combination and comparison functions such as sums, averages, differences, and correlations. A useful comparison metric may be the difference between an activation metric for a recent period of time and the same activation metric computed for a reference period of time, such as an earlier period of time. This value indicates the changing level of activation in an ROI. The difference can also be computed between the average value of an activity metric computed from one time period, such as the difference between the average of a metric for all time points falling within a particular behavioral or stimulation condition, or for all time points falling within a background or rest condition. Combinations can also be made between separate activity metrics, including such as sums, averages, differences, and correlations. An example is the difference in activation level between one ROI and another ROI at the same time point. Differences can also be computed for different time points, which can be useful in determining whether one area is leading or lagging another area.

E. Normalization of Activity Metrics

Once activity metrics have been computed for voxels within the brain, those metrics may be normalized based upon the type of tissue that they arise from. An example of normalization of activity metrics is to divide the metrics computed for each voxel by a constant value that applies to the tissue type of that voxel. For instance, voxels corresponding to gray matter may have a normalization value that corresponds to greater levels of variance, while voxels corresponding to white matter may have a normalization value that corresponds to lesser levels of variance. Similarly, other types of normalization may be used that depend upon predicted levels of each activity metric for different types of tissues. Average values of activity metrics have been developed for different regions of the brain (such as the cortex, thalamus, Brodmann areas, or other structures), and/or for different subject groups (such as older or younger subjects or subjects with a particular medical condition) may be used to normalize the measured activity metrics from individual measured voxels within the brain.

F. Displaying Activity Metrics

Activity metric data may be presented using a display or stored for later use, transmitted electronically, or printed out. The resultant images may be presented in a variety of ways, as described in the examples presented in the following section. These will be familiar to one skilled in the art.

2. Diagnostics of Functional Status or Abnormality

An important aspect of the present invention is the measurement of the functioning of neural tissue. In one example, this functioning is measured while a subject is at rest (not performing a task). This measurement is made using measures of the variance of activation at a voxel, standard deviation of activation at a voxel, coefficient of variation of activation at a voxel, or correlation of activation between two voxels. These measures may be computed for multiple voxels in order to form images. These images may be used to note regions with either larger or smaller values than other regions. These measures may then be interpreted as an indicator of physiological functioning at those voxels.

Brain tissue in healthy individuals shows continuous variations in its fMRI signal, and these variations are correlated with tasks, and correlated among brain regions. These various methods provide differing information regarding tissue functional status. Anatomical structures may have consistent task-activation, fluctuation and correlation that may be used to assess functional status. Measures may include task activation, coefficient of variation maps, functional connectivity maps, and distributed connectivity maps.

A. Coefficient of Variation (CV) Maps

The voxel-wise coefficient of variation may be mapped for each subject in the differing conditions. This index gives the total fluctuations undergone by each voxel in an image or volume as a percentage, normalized by the voxel's mean value. CV maps may be generated for different frequency bands by band-pass filtering data before processing. Large amplitude components present in CV maps in high frequency bands are often associated with vascular elements. Following segregation of tissue into cortical gray matter, white matter, and by anatomical structure, average CV measures will be derived for each structure.

B. Functional Connectivity Maps

Maps of the correlation coefficient computed between single seed voxels or regions of interest and remaining brain voxels may be computed using seed voxels (or data from larger ROI's) in anatomically-defined areas such as primary motor cortex, primary visual cortex, language areas (Brodmann 22, 44, 45). These maps may be computed using both the standard correlation coefficient method, and using a measure of correlated fluctuation. The correlated fluctuation measure is the CV at each voxel multiplied by the fraction of variance at that voxel accounted for by the linear correlation with the seed voxel (or ROI) using a pair-wise linear r2 measure. This measure maps the extent to which a voxel has large magnitude fluctuations in correlation with a seed voxel.

C. Distributed Connectivity Imaging (DCI)

Correlation (or functional connectivity) maps typically provide a measure of the correlation between a single voxel (or ROI) and all other brain voxels tested. This determines the functional relationship between particular anatomical areas in a pair-wise fashion. However, this method provides a map of correlation from a single location at a time. It is also useful to have a global map of the correlated variance at all voxels, not taking a single region as the seed to generate a correlation map, but taking each voxel as the seed voxel in succession. This may require a data reduction given the all-to-all correlation matrix of data. A connectivity index may be computed that collapses the measure of correlated variance between each voxel and all other voxels. This measure may be the product of the multivariate linear regression R2 between a voxel and all other voxels, and the variance at the given voxel. It may correspond to the magnitude of fluctuation correlated with activity elsewhere (normally excluding an immediately surrounding region). Once this connectivity index has been computed for each voxel, a simple map may be generated, a distributed connectivity image.

D. Assessment of Normal vs. Abnormal Function

The normal or abnormal function of tissue may be assessed by comparing the value of an activity metric, such as one described in A-C, with a reference value. The reference value may be the value in a contralateral brain region from the same subject. The reference value may be the value in a different brain region in the same subject. The reference value may be an average value from multiple brain regions from the same subject. The reference value may be the value in a contralateral brain region from a different subject, or the average form a group of subjects. The reference value may be the value in a different brain region from a different subject, or the average form a group of subjects. The reference value may be an average value from multiple brain regions from a different subject, or the average form a group of subjects. In this way, it may be determined whether the function of an area is normal or abnormal. Abnormal function is defined as having a measure outside of the normal range measured in a group of subjects, or significantly different compared with a reference value.

In order to perform this assessment, data from many subjects with different conditions may be collected and stored. Additional information regarding various conditions that these stored subjects had or did not have may also be stored. In this way, by comparing the values of a measured activity metric from a brain region from a particular subject with values taken from subjects with or without a condition, it is possible to determine whether the value for the subject is more similar to those prior subjects with the condition or those without the condition, and thereby to assign a likelihood that the subject has the condition.

3. Using fMRI to Create a Diagnostic for Normal or Abnormal Function

Many clinicians, including several participating in this study, have stressed the substantial potential importance of being able to derive images to localize functional abnormalities in brain tissue during a routine MRI exam[1]. Brain MRI scans to detect anatomical abnormalities such as masses or other lesions are conducted in more than one million patients per year, and in the majority of cases there would be great additional benefit from deriving simultaneous images showing any functional abnormalities. Innovative computational and bioinformatics methods to make this possible using fMRI data are described in this invention. Some of these methods require the performance of a task. Others use fMRI data collected while a subject rests passively in the scanner, and could therefore be applied in cases where the performance of cognitive tasks may be impossible or impractical, such as in assessing cognitively impaired patients or infants. In this example, a subject may receive a T2*-weighted functional MRI scan, possibly in addition to T1-weighted, T2-weighted or other imaging sequences. The T2* data may be used to measure variance, correlation, or functional connectivity. These measures may be presented as images, or compared between structures within the subject, or compared with other subjects. These values may be interpreted as indicators of the functional status of corresponding brain regions. These values may be interpreted as indicators of the presence of abnormal function or disease. For example, images of this sort may be used to outline the region of brain that is no longer functioning normally that may include or surround the area of a stroke or traumatic brain injury. These measures may also be used to determine the likelihood of presence of neurological diseases such as epilepsy, alzheimer's disease, Parkinson's disease, or other disorders as listed elsewhere in this application.

4. Motion Correction and Cardiac/Respiratory Correction Methods for fMRI

A. Respiratory and Cardiac Noise Correction

In order to measure correlations in fMRI signals, it is important to reduce noise to the extent possible. (Glover, G. H., T. Q. Li, and D. Ress, Image-based method for retrospective correction of physiological motion effects in fMRI: RETROICOR. Magn Reson Med, 2000. 44(1): p. 162-7.). Briefly, a simple image-based correction method is described that does not have the limitations of k-space methods that preclude high spatial frequency correction. Low-order Fourier series are fit to the image data based on time of each image acquisition relative to the phase of the cardiac and respiratory cycles, monitored using a photoplethysmograph and pneumatic belt, respectively. Also, collection of inter

B. Motion Correction

fMRI data can be corrected for subject motion in a number of ways. This is useful before processing of data, particularly for single-subject and correlation measures. Motion may be corrected using prospective methods (Thesen, S., et al., Prospective acquisition correction for head motion with image-based tracking for real-time fMRI. Magn Reson Med, 2000. 44(3): p. 457-65. Ward, H. A., et al., Prospective multiaxial motion correction for fMRI. Magn Reson Med, 2000. 43(3): p. 459-69.) or retrospective methods such as those employed in existing software packages such as AIR, AFNI, SPM99, Brain Voyager. In addition, anatomical data may be collected in an interleaved fashion with functional data, using scanning and pulse sequences to collect T1 and T2* data, so that precise motion correction may be acheived.

5. Examples of Information Displays

As has been noted, an important aspect of the present invention relates to the provision of information to the subject as the subject's brain activity is measured in order to influence and test how the subject performs during testing exercises. In one variation, information is communicated to the subject through computer generated displays which the subject is able to observe during testing.

The information can relate to instructions, brain measurements, sensory stimuli, and testing performance. Each of these different types of information may be displayed by itself or in combination with other types of information.

The layout of the content of the information displayed can be widely varied. For example, the information can be in graphical and/or in text form. The displayed information can include static images as well as moving images, and optionally can also be accompanied by sound, or by other forms of sensory stimulation. The subject or device operator can select multiple types of information that will be displayed together from among those described and depicted here.

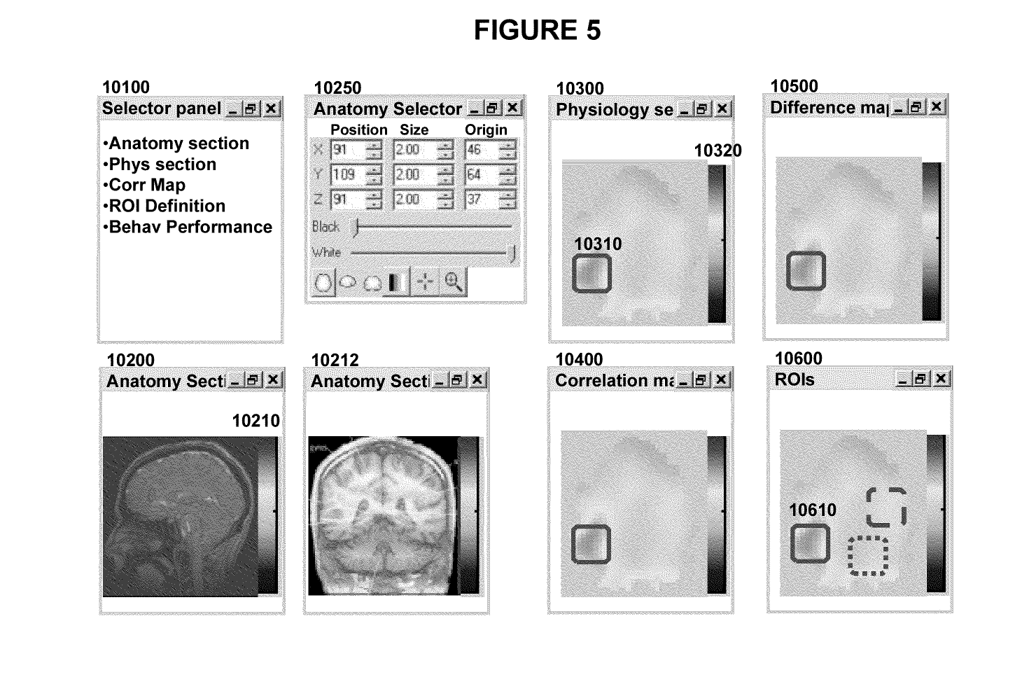

Described herein are examples of what types of information may be displayed to assist the subject. Example display panels are shown in FIGS. 4-5.

A. Instructions

An important type of information that may be displayed to a subject is instructions. These instructions alert a subject regarding different things that the subject is asked to do including perform a testing exercise, rest and other forms of response that may be asked of the subject. The instructions may be displayed concurrently with other forms of information.

Moving visual images or a sequence of sounds or verbal instructions or other means of communication can instruct the subject to perform ongoing sequenced behaviors, with each successive element in the sequence controllable based upon measured physiological activity. Provided herein in are examples of different instructions and ways of communicating brain measurements that may be displayed.

B. Stimuli

Another important type of information that may be displayed to a subject is stimuli. Provided herein are examples of different ways of communicating stimuli. Types of stimuli that may be presented include static or moving visual displays, tactile, proprioceptive or heat stimuli, odors, sounds, and other forms of sensory information.

6. Remote Processing of Data

It should be noted that the various computational and data processing steps inherent in this invention need not be performed on devices located in the same place, or even in close proximity. An example of remote storage and processing of data, is that subject brain scan data may be transmitted via the internet either during testing or following testing to a remote site where the process of data analysis takes place. The processing of this data at a remote location may be provided as a service, with results of the processing being transmitted back to the subject's site, or two additional sites such as the site of a subject's physician, or others involved with the procedure. Another example of remote storage and processing of data, is that a database of information of brain activation from a number of subjects may be stored at a remote location, and the activity metrics from a subject may be compared with the information in this database, as described in section 7 on the comparison of a subject's data with group data. This process may be provided as a service from a centralized point including computers that can perform this data processing, and means of receiving subject input information and transmitting the resultant output, such as via the internet, as well as means for storing information. Another example of remote storage and processing of data, is that the software that performs some or all of the processing steps involved in this invention, including reconstruction, motion correction, pre-processing, computation of activation images, or computation of activity metrics, may take place at a site remote from the subject. Once again, this process may be provided as a service from a centralized point including computers that can perform this data processing, and means of receiving subject input information and transmitting the resultant output, such as via the internet, as well as means for storing information. Another example of remote storage and processing of data, is that an application service provider model may be employed, whereby processing means or data is provided via the internet or other means to the site where the processing needs to be accomplished from one or more centralized distribution points. This processing means and data may then be used locally to process subject data into activity metrics, and to compare this data to one or more existing databases of information. One skilled in the art will recognize that some or all of the data and processing means involved in this invention may either be resident on a machine in the location where the subject is being scanned, may be present at a remote location where processing takes place, or may be provided transiently to the location of the subject at the time of processing using an application service provider model. This process may all take place via web browsers. Additionally, patient information such as clinical information, other demographic information, treatment information, or other medical records may be transmitted to the remote location to be processed and/or stored along with the subject's scanning information. Similarly, the behavioral control software that the subject interacts with when within the scanner may be resident a the location of scanning, may be resident at a remote location with viewing of output via the internet or some other communication means (such as viewing of stimuli or instructions for the subject in a web browser), or may be provided as an applet or some other form of served application.

7. Modes of Communication with a Subject

A variety of different modes of communication can be used to relay information between the subject and another party, for example a medical professional. For example, information may be communicated between people, transmitted through a direct electrical connection to a nearby point, or through a connection mediated by land-line or wireless telecommunications equipment or the internet. Various examples of how information may be communicated in the system of the present invention are provided below.

A. Two Way Audio and/or Video Communication

According to this variation, the voice of the subject is picked up using a microphone within the apparatus, transmitted, amplified, and played to the device operator or other healthcare professional, either nearby or distant. This recording can be turned off automatically or manually during the process of scanning. The voice of the device operator or other healthcare professional is picked up using a microphone, transmitted, amplified, and played to the subject. In some instances, one-way or two-way video communication is also used by imaging the patient in substantially real time and presenting the image to the device operator or other healthcare professional, or imaging the device operator or other healthcare professional and presenting the image to the subject in substantially real time on the monitor viewed by the subject.

B. Subject Control of Computer Interface

According to this variation, a computer interface is provided that allows the subject to input information. A wide variety of input devices are known, including, but not limited to computer joystick, mouse, trackball, keyboard, keypad or touch-screen, a botton-box with response buttons that the subject can press, game controller devices, and other computer interface means. These devices can also allowed shared control of a pointer or cursor on a computer with a pointing device controlled by the device operator, such that either device can be used to control the pointer or cursor.

8. Decoding of Brain Representation

An important objective is to be able to ‘decode’ activation in the brain, in order to gain insight into the behavior, mood, emotion, motivation, motor intention, sensory perception, or other aspects of a subjects cognitive processing. This invention may be used to decode neural representations. In order to accomplish this, activity patterns are associated with cognitive processes. Then, when an activity pattern is measured, this serves as an index into the likelihood of the presence or the magnitude of a cognitive process that is taking place. In one version, the

A. Types of Decoding Strategy

In order to decode neural activation, it is necessary to form a relationship (or model) between observed patterns of brain activity and cognitive processes, states or events. This can take place in two modes:

i. Categorical Relationships

In categorical relationships, the presence of a particular pattern is presumed to correlate with the presence of a state of cognitive processing. For example, the presence of above-average activation in a brain area associated with language processing can serve as an indication that a subject is processing language information.

ii. Magnitude Relationships

In magnitude relationships, the magnitude of a particular pattern is taken to correlate with the magnitude of a particular cognitive process. For example, the magnitude of activation in a brain area representing one area of the visual field may serve as an indication of the magnitude of the subject's attention to that area of the visual field.

B. Determining a Decoding Strategy

The relationship between a pattern of activation and a cognitive process is arrived at through repeated observation of their co-occurrence. This can take place through several modes:

i. Forward Measurement

In forward measurement, a given cognitive process is generated in a subject, for example by the presentation of a stimulus for perception, or through inducing a subject to undertake a behavior. The pattern of activation associated with that cognitive process is then observed. This process is repeated until a relationship has been determined for one or more cognitive processes and one or more patterns of activation. Then, when a pattern of activation is observed, it can be inferred that the corresponding cognitive process is taking place. For example, if a visual spot is successively placed in many positions in the visual field in order to measure the resulting location or pattern of activation for each location, then when a given location or pattern of activation is observed, it can be inferred that the corresponding area of the visual field is being perceived, or that processing involving this part of the visual field is taking place, as in the case of attention to this part of the visual field, or mental imagery including this part of the visual field. Examples of this approach using measurements from single neurons in animals have been described in (Britten, K. H., et al., The analysis of visual motion: a comparison of neuronal and psychophysical performance. J Neurosci, 1992. 12(12): p. 4745-65. Salzman, C. D. and W. T. Newsome, Neural mechanisms for forming a perceptual decision. Science, 1994. 264(5156): p. 231-7., 1 Hubel, D. H. and T. N. Wiesel, Receptive fields, binocular interaction and functional archtecture in the cat's visual cortex. J. Physiol., 1962. 160: p. 106-154). Conceptually similar approaches may be employed in this invention.

ii. Reverse Correlation

A second method for determining the relationship between brain activation and cognitive process is reverse-correlation. In this method, a pattern consisting of a variety of stimuli is presented, typically with different semi-random patterns of multiple stimuli being presented at the same time. Once many patterns have been presented, a measure of neural activation, such as the activation level at a single voxel, may be reverse-correlated with the time of presentation of a particular stimulus. For example, a checkerboard in polar coordinates or Cartesian coordinates may be presented to a subject, with each checkerboard location independently changing color at semi-random time increments. Then, the average activation at a given brain voxel may be computed for time periods following when each location in the checkerboard was in a given state (for example color). In this way, a correlation may be generated between a given activity level in a given brain voxel, and a probability that a given location in the checkerboard as in a given state (for example white). Normally, a given brain voxel will be activated in correlation with a limited range of areas of the visual field, called the receptive field. This same process may be performed using a range of different sound frequencies, a range of different tactile sensation locations, or a range of different movements. This process has been described for forming correlations between single neurons in animals and stimuli (deCharms, R. C. and A. Zador, Neural representation and the cortical code. Annu Rev Neurosci, 2000. 23: p. 613-47. deCharms, R. C., D. T. Blake, and M. M. Merzenich, Optimizing sound features for cortical neurons. Science, 1998. 280(5368): p. 1439-43.), and the process may be directly extended for use in the present invention by replacing a single neuron measure with an activation level or activity metric, as will be clear to one skilled in the art.

Once this process has taken place, it is possible to form maps of the correspondences between spatially distributed voxels, and spatially distributed referents (such as the visual spatial field). Adjacent brain sites generally correspond with nearly adjacent regions in perceptual space (e.g. adjacent points in visual space, adjacent points on the body for tactile sensation, adjacent sound frequencies, similar movements, similar conceptual processes, similar emotional states, similar words or ideas). Therefore, it is possible to create representational maps.

C. Decoding the Content of Neural Representation

Following the forward or reverse correlation process, detail predictions may be made of the representational content inherent in the activation of a single voxel, or complex patterns of activation encompassing multiple voxels. This takes place by taking the joint predicted (or decoded) cognitive process associated with the activation level at each voxel, and combining them to produce an overall estimate of cognitive processing taking place at a given moment. This process may be used to infer the cognitive processing of a subject. This may be useful in allowing the subject to communicate directly through their cognitive processes, when those processes are decoded as described in this invention. This may be useful in other contexts when it is relevant to be able to estimate the cognitive processes of an individual, such as in the context of a lie-detector test.

9. Assignment of Structures Through Anatomical Registration

Each voxel can be probabilistically assigned to an anatomical structure by registering the brain of a subject with a standard reference brain according to standard methods. In this way, the values of any activity metric may be derived for an anatomical structure by collecting the measures from all voxels corresponding to that structure.

10. Sound Cancelling Headphones

In order to increase patient comfort within the scanner, which can be loud when operational, subjects may be provided with sound cancelling headphones. These headphones can be used to produce an opposite waveform to the sound produced by the scanner. This can be accomplished by using a microphone close to the subject to measure recorded sound, and providing an appropriately amplified complementary signal to defeat the sound heard by the subject. Equipment designed for the purpose is, for example, the Instructioner produced by Resonance Technology, CA.

Sound cancellation can also be accomplished by providing an amplified, digitized, pre-recorded waveform to the subject that is substantially the opposite of the repeated sound waveform produced by the scanner. The subject or device operator is then allowed to adjust the delay of this repeated signal with respect to the scanner noise and the amplification of this signal so as to produce the maximal sound cancellation.

This signal may be presented using either headphones worn by the subject, or using headphones or earplugs with sound-conductive tubing that lead sounds to the subject's ears from a speaker outside of the measurement apparatus.

11. Localization of Structures Using Standard Coordinates, and Coordinate Transforms

This section describes several ways in which one may localize regions of interest from on physiological scan data. If a given anatomically-defined region is to be used as the region of interest for a subject, software may be used to select the voxels of a given subject's physiological and anatomical brain scanning volume corresponding to that anatomically-defined region. This selection may take place in substantially real time. For example, the user may select an anatomical region of interest from a pre-defined database of anatomical regions. Software may then be used to determine the voxels within the physiological or anatomical scans of the subject that correspond to the selected structure. The software can also highlight the structure, draw an outline around it in 2-D or 3-D representations of the subject's brain, and label the structure. The software can also be used to label all structures on a given section of the subject's brain, or all structures that match a selected criterion, such as all cortical areas. The software can also use custom anatomical boundaries defined by the user, which can also be added to this database. Examples of this functionality are shown in FIG. 4-5.

The first step in this process is for the device operator to select the anatomical area of interest from a standard coordinate system brain, such as the Talairach Atlas or the MNI Atlas with corresponding coordinate system. The device operator can do this by using a text designation of the area of interest (such as a particular Brodmann's Area). This text designation can be either selected from a pull-down menu of pre-defined choices corresponding to the anatomical areas taken from an atlas plus user-defined areas, or entered as free text. This text designation is searched from a database of which voxels correspond to which anatomical areas to produce a list of corresponding voxels. Additional areas defined in the same way can be added to create a combined area, or subtracted to create a difference area. Alternatively, the user can select the region of interest from one or more planes of an anatomical map in standard coordinates. These selected voxels from the standard brain can be saved to disk as a brain volume mask, or as a list of voxel points, and used at the time of scanning.

The transform from standard coordinates to the coordinates of a particular subject being measured must then be defined. This takes place by the user designating a variety of points on the subject's brain that will be used to correspond these points to the pre-defined standard coordinate brain. The first point selected will normally be the anterior commissure, shown on a mid-sagittal section. The program will assume that the subject's brain is identical to the standard coordinate brain, and present on the display the point corresponding to the anterior commissure in a standard brain as a target on top of the section of the subject's brain as a background, while also presenting text designating the name of the structure: “anterior commissure”. The device operator can select a different section as the background section. The device operator then mouse-clicks the point of the anterior commissure on the actual section of the brain of the subject as seen in the background section. The program will take in the point of the anterior commissure in 3-D coordinates, so that it can be compared with the reference brain point. The difference in position between the point in the standard coordinate brain and the point measured for the subject's brain is added to subsequent points before they are displayed to the subject, to shift the display point to be closer to that observed for the subject. The program will then go through a variety of additional points in succession and present targets for the point on the subject's brain; the user will select the point of the anatomical location on the subject's brain; and the program will take in this data. The targets are used so that the user may more quickly select each corresponding point on the subject's measured brain volume, without reading a text description of the relevant area to select. The points used will include: anterior commissure, posterior commissure, occipital pole, frontal pole, rostral pole (normally all selected on a mid-saggital section), left and right extremes of brain (normally selected on a coronal or axial or horizontal section). Additional points can be used for an even better fit. Once the locations of all of these points in the standard coordinate brain, and in the measurements for the subject's scan volume, the 3-D to 3-D affine transformation is computed using standard methods that produces the least-squared error in transforming the points in the standard coordinate brain to the points in the subject's observed brain volume. This transformation takes into account translation, rotation, and scaling to locate corresponding points within the subject's physiological or anatomical scanning volumes with those from the standard coordinate brain. This transformation will be used to make the correspondence between all other points. This process can take place while the subject is in the scanner, in a matter of seconds or minutes from the time the data is actually collected, and using the same computers and software used in the scanning and substantially real time data transformation procedures.

If necessary, more complex transforms can be computed, including internal morphing to allow more precise correspondence between defined anatomical points within the two structures with interpolation of the correspondences of points intervening between the defined anatomical points. Also, the transformation can take place by automatic registration of brain volumes (see for example methods described in SPM99 and other existing MRI/fMRI/PET data processing packages).

Once the transformation has been determined, any point in the standard brain can be translated to find the corresponding point(s) in the subject's brain scan volume, and vis. versa. Therefore, a volume mask is generated corresponding to every point in the subject's brain volume that corresponds to a point from the anatomical structure(s) selected by the device user. This volume mask can be overlayed upon the subject's brain images to allow the user to more easily and accurately select the location of a region of interest, or the volume mask can be used as a region of interest itself.

Each voxel in the subject's brain can be assigned a fractional probability of being within a defined brain structure. To do this, all of the points from the standard brain that correspond to a given point in the subject's measured brain volume are determined, along with the fraction of overlap, which is used as a weighting factor. The fractional probability of being within a given structure is then determined as the sum of (the product of each corresponding pixel's being within that structure as determined from existing atlas data, times that pixels weighting factor.)

The software can function in the reverse direction, providing a spatial readout of the location in standard coordinate space of a given location in the brain of a subject selected by the device operator on a screen display, based upon reverse the vector transform. In addition, the resultant location in the standard coordinate space can be used to perform a lookup function within the 3-D database in order to produce the name of the anatomical structure at the corresponding location. Finally, the anatomical boundaries of the structure selected within the subject's brain can be drawn and labeled as a contour map surrounding all voxels included within the structure, or having a threshold probability of being within the structure.

12. Summary of Scanning Protocol

In this section, an exemplary scanning protocol is provided. It is pointed out that this protocol is for illustration purposes and may be modified as has been described in the other sections. It is also pointed out that aspects of this protocol are directed to performing a fMRI scan. Modifications to the protocol are within the level of skill in the art for other brain scanning methodologies.

After pre-scanning testing has been performed, subjects are first placed in the scanner, and a series of scans take place over a period of minutes or hours.

T1-weighted saggittal localization scans are conducted to localize the brain precisely and achieve registration.

T1-weighted anatomical scans are also conducted to precisely image the brain and central nervous system

Functional scan(s) may then be performed to localize the regions of interest. During these scans, the subject may be asked to perform a task alternating with rest periods (with each typically lasting about 30s). After this has been repeated 3-20 times, the average activity may be computed for each voxel within the brain or other body zone in order to determine the region(s) of interest as described above. During this process, the subject observes images of the activity pattern within their brain so that they learn what the activation achieved by a behavior in a particular region looks like, and are encouraged by their success.

Initial testing scanning is then performed to test the subject in how to control a brain region. The subject can be asked to control a region of the brain that is ‘easier’ to control than the ultimate testing target so that they learn how to accomplish this and build confidence. In one embodiment, subjects are asked to alternatively activate and inactivate their functionally defined primary motor cortex digit representation of one hand by imagined hand movement. The subjects learn how to control this brain region and are rewarded for their correct performance.

The subject may be given a ‘control task’ which is identical to the task described below, except that the information presented to the subject does not give accurate information about the state of activation of their brain. The information presented comes from another (pre-recorded) subject, from a different brain region than the one being considered, from an earlier time, or a combination. In one embodiment, the subjects may be given ‘sham feedback’ which they are told comes from the region of interest the second before, but actually comes from another brain region 30-60 s before. This allows the clear determination that subjects are using the information being presented to them to control their brain activation (in comparison with this control case where they are not).

The subjects may be given multiple testing periods of many trials or continuous testing. The subjects are shown the screens described above, and asked to perform many trials at the times cued. In each trial, the subject alternated between performing the desired task and resting or performing a different task. The subject is instructed to achieve the desired pattern of brain activation. In one embodiment, this desired pattern is an increase in activation in a defined brain region during the task period compared with the control period. As the subjects progress through the trials, in one embodiment an adaptive tracking procedure is used to aid in their testing. This procedure sets a target level of activation for each trial based upon the level achieved in recent trials (using a psychophysical 3 up, one down procedure). As the subject does better, the trials become more challenging. If the subject begins to make errors, the trails become easier. The subject is given both continuous immediate information about the level of activation in the relevant brain region, as well as information about their behavioral performance. This testing takes place either using the alternating methodology described, or with the subject's objective being a continuous increase in activation of the target region, or replication of the intended pattern.

The subjects are then given test periods to simulate being outside of the scanner. On certain trials, or periods of trials, subjects are not provided with information about the level of brain activity, and they are tested to determine whether they are nonetheless able to produce the desired modulations. This simulates the situation that the subject will encounter in controlling their brain activation state when no longer in the scanner, and allows the evaluation of their success.

13. Scanning Parameters

For fMRI, an example of scanning parameters that may be used is as follows. It is noted that one of ordinary skill will know how to perform fMRI and thus will know how to deviate as necessary from these parameters.

Scanner fields can range from 0.1-10 Tesla or more. Scan volumes can range from 1 mm to 40 cm, and can be divided into voxels with edge sizes from 1 micron to 20 cm. Scan repeat rates can be 0.01 to 1000 Hz. TE can range from 1-1000 ms, and TR can range from 1-4000 ms.

14. Contrast Agents

It is noted that contrast agents may be optionally used in combination with fMRI for physiological signal measurement when performing the various methods of the present invention. By using contrast agents to assist brain scanning, it may be possible to achieve larger and more reliable activation measurements than using tradition BOLD signals which rely on endogenous contrast particularly as provided by hemoglobin. Examples of exogenous contrast agents that may be used in conjunction with the methods of the present invention include, but are not limited to the contrast agents disclosed in U.S. Pat. No. 6,321,105.

15. Background Conditions

Background conditions for testing and measurement are used to set the ‘baseline’ level of a localized brain region's activation, or another activity metric. Further measurements can be made in comparison to this baseline. For example, a subject might be tested while increasing the level of activation of a localized brain region above a baseline level, and that baseline level might be determined by the activation of that region when the subject is resting and not performing a task. If a different baseline level is chosen, such as the level when the subject performed an alternative task, then the increase above this alternative baseline level would be different. Frequently, the activity pattern measure of interest is the difference in activity between a task state and a baseline level measured for a background condition. Therefore, it is important to select an appropriate background condition.

As was described previously, the simplest background condition is typically a rest condition during which the subject is not explicitly instructed to perceived particular stimuli or perform particular behaviors. However, there are circumstances and brain regions for which ‘rest’ can still produce significant levels of activation. For example, if at ‘rest’ the subject tends to engage in cognitive activities such as internal dialog or other types of thoughts, there can be activation of certain brain regions associated with these cognitive activities, such as in the frontal lobes. The activation during a background condition may also be compared with the activation observed during an intervention.

More complex background conditions are designed to selectively deactivate a region of interest, or to activate other regions than the region of interest. For example, a background condition for a verbal mental rehearsal task is the task of imagining mental images in the absence of internal verbalization. This background condition may lead to a lower or different pattern of activation in the region of interest, such as in the region responsible for verbal mental rehearsal. This background condition may also lead to an increase in activation in other regions, such as occipital and frontal regions responsible for internal visualization. Other background conditions include tasks that will inhibit subjects from engaging excessively in unrelated thoughts, such as a simple reaction time task or a task require select which stimulus was presented of several possibilities. In some instances a background condition to measure a truly low level of activity could be one of the various states of sleep such as slow wave or REM sleep, anesthesia, or other reduced level of awareness.

16. Head Motion Stabilization

For many of the brain scanning technologies, it is important for the subject's head to be kept stationary. This becomes an issue when the subject is tested for an extended period of time. Accordingly, the present invention also relates to devices reduce head movement. Movement cancellation software and technologies may allow less restrained head movement or free head movement during measurement using this invention.

In one embodiment, the subject is placed within a head restrained system similar to the type used following cervical spinal injury. The restrained system may be anchored or placed in such a way as to ensure stability, minimize motion, and allow reproducible placement of the head in space within the scanner on successive occasions. The restrained system preferably is able to conform to a shape of the head and neck of the subject and may include adjustable straps to hold the head securely within the device. The materials used may be semi-rigid or a combination of hard materials coated with softer material to make them comfortable, with all materials being scanning transparent.

In another embodiment, a custom-fitted head mold is provided to hold the head of the subject stationary. This mold is preferably removeably attachable to the scanner so that the mold may be immobilized relative to the scanner. The mold may be created through injection molding using a lightweight, largely rigid yet somewhat soft, and scanning-transparent material such as styrofoam to form a mold shaped to fit all or part of the subject's head, neck, and upper torso. Optionally, the subject's head motion may be additionally stabilized using a bite bar that is placed to allow the subject to embed his/her teach within the material and thereby maintain a fixed position.

For some applications, such as fMRI, it is desirable to precisely position the subject's head, for example relative to the scanner scanner or head coil. This positioning of the head may be accomplished by placing the subject in the scanner so as to precisely locate points on the head by matching localization points with physically constant or precisely adjustable locations attached to the scanner or head coil. In one variation, large plastic or other screws are threaded through holes in the apparatus holding the subject and adjacent to the head may be used. These screws may be screwed in until they just touch the head of the subject, with the number of turns providing a precise a reproducible measure of the location of the point on the head. The screws can also be formed with soft pads attached to their ends that serve to restrain motion of the head. Conventional neurological ‘halos’ can be adapted to this purpose.

17. Cardiac and Respiratory Gating

Some portions of the brain undergo significant movement as a result of the cardiac cycle as well as respiration, and these movements introduce noise into physiological signals measured from the corresponding scan volume voxels. The present invention can be used in combination with techniques that decrease the impact on measured physiological data of physiologically-based motion such as cardiac motion and respiratory motion. One technology that may be used to decrease the observed motion of certain brain regions is cardiac gating. Brain measurement times are triggered by measurements of the timing or phase of the cardiac rhythm cycle so that, on average, successive brain measurements are taken at substantially the same point in the cycle with brain regions in substantially the same position. For instance, the start of each cardiac cycle is detected using an EKG or pulsoxymetry device, and this time is used to trigger the presentation of an MRI RF pulse sequence and ensuing measurements.

Another technology that may be used to decrease the observed motion of certain brain regions is respiratory gating. Brain measurement times are triggered by measurements of the timing or phase of the respiratory rhythm cycle so that, on average, successive brain measurements are taken at substantially the same point in the cycle with brain regions in substantially the same position. For instance, the start of each respiratory cycle is detected using a pulsoxymetry device, and this time is used to trigger the presentation of an MRI RF pulse sequence and ensuing measurements.

18. Measurement of Activity

This invention may be used in conjunction with a variety of means for measuring physiological activity from a subject. Examples of measurement technologies include, but are not limited to, functional magnetic resonance imaging (fMRI), PET, SPECT, magnetic resonance angiography (MRA), diffusion tensor imaging (DTI), SSFP, parallel imaging (e.g. SENSE), trans-cranial ultrasound and trans-cranial doppler shift ultrasound. It is anticipated that future technologies may be developed that also allow for the measurement of activity from localized brain regions, preferably in substantially real time. Once developed, these technologies may also be used with the current invention. These measurement techniques may also be used in combination, and in combination with other measurement techniques such as EEG, EKG, neuronal recording, local field potential recording, ultrasound, oximetry, peripheral pulsoximetry, near infrared spectroscopy, blood pressure recording, impedence measurements, measurements of central or peripheral reflexes, measurements of blood gases or chemical composition, measurements of temperature, measurements of emitted radiation, measurements of absorbed radiation, spectrophotometric measurements, measurements of central and peripheral reflexes, and anatomical methods including X-Ray/CT, ultrasound and others.

Any localized region within the brain, nervous system, or other parts of the body that is measured using physiological monitoring equipment as described (or other physiological monitoring equipment that may be devised) may be used as the region of interest of this method. For example, if measurement equipment is used for the monitoring of activity in a portion of the peripheral nervous system, such as a peripheral ganglion, then subjects may be tested in the regulation of activity of that peripheral ganglion. In addition, this invention may be used to monitor the blood, blood volume, blood oxygenation level, and blood flow in the vasculature of the brain and other bodily areas, which may serve as regions of interest.

19. Behavioral Testing

Using this invention, subjects may be tested in a variety of tasks. Testing corresponds to performing a task with the intent to improve or test a desired outcome, and is typically repeated. Tasks may include covert behavioral tasks in which a subject performs a cognitive or mental activity such as imagining a movement in order to activate a brain region, or overt behavioral tasks in which a subject performs a physically observable action such as making a prescribed movement or responding to a question. Tasks may also include the administration to the subject of an intervention. In either case, the task may lead to changes in the activity of the brain of the subject, and these changes may be measured as provided for in this invention. Overt and covert tasks may be performed separately, or substantially concurrently.

One example of behavioral testing is covert testing of a subject to activate a brain region of interest. In this example, the subject may be provided with information about the level of activity in a brain region of interest, such as an activity map including the region, or an activity metric that measures the activity in the region of interest. This testing may be with the intent of increasing the activity in the region of interest, decreasing it, changing its pattern, or altering it in other ways as measured by the activity pattern metrics described in Examples section 1. The subject may also be presented with stimuli, which may additionally serve to activate a brain region of interest. The subject may also be presented with performance information indicating his or her level of performance at the task being performed. The subject may monitor these types of measured information, stimuli, and performance information, and may respond to them. One response of the subject may be to select or modify a cognitive strategy that the subject uses to activate the brain region. For example, if the subject is performing the covert task of imagining a given hand movement in an attempt to activate the motor cortex, the subject may observe that one particular imagined hand movement is more effective at activating the motor cortex than another particular imagined hand movement. The subject may then select the more effective movement for use in future trials. This monitoring of information and response may take place in combination with performing testing. While the results of a covert task may be observed using physiological measurement equipment, they are not observable in the sense of producing an overt, physically observable, visibly viewable action of the subject.

Another example of behavioral testing is overt testing of a subject to perform a physically observable, overt task. The subject may engage in overt tasks such as psychological, learning, motor, or psychophysical tasks. These may include such as things as making a computer selection of which of two stimuli presented has a particular feature, or making a prescribed motion, or answering a stated question. The subject may additionally be given performance information regarding their performance at these covert tasks, such as whether they performed tasks correctly or incorrectly. The performance of covert tasks may take place substantially concurrently with overt tasks. For example, the subject may be instructed to make selections between different stimuli or to perform particular movements while the subject also attempts to increase the level of activation in a brain region of interest.

20. Target Brain State Testing

The present invention may be used to perform target brain state testing where a subject is tested on achieving a selected target brain state of activation. A target brain state of activation may be a spatial activity pattern within a region of the brain, a series of regions of the brain, or the entire brain.

As an example, a method is provided for testing a target state of activation comprising: selecting a target state of activation in one or more brain regions, measuring a current state of activation in those regions, comparing the current state of activation to the target state, providing information about the measured comparison, and providing for testing with knowledge of the comparison as a guide to reducing the difference between the current state of activation and the target state.

By knowing how the current state of activation compares to the target state, testing may be selected and/or modified so that the target state is achieved. Because information regarding the current state of activation and the comparison may be determined and communicated to the subject or device operator in substantially real time, testing may likewise be selected and/or modified in substantially real time.

Comparing the current state of activation to the target state may be performed by software that determines a difference between the current and target state. For example, software may be used to compute a vector difference, vector distance, or a dot product between two spatial patterns of physiological activity, namely the spatial patterns of the current spatial activity pattern and the target spatial activity pattern. For example, an activity metric may be computed that measures the difference between the current activity pattern in a region of interest and a target activity pattern.

The target and current states of activation may each be expressed as representations of an absolute level of activation in a number of brain regions. Accordingly, comparing the states involves comparing these representations.

The target and current states of activation may also each be expressed as representations of which regions have a desired increase in activation, and which ones have a desired decrease, with magnitudes of increase and decrease being optional. Again, comparing the states may involve comparing these representations.

A. Selecting a Target Spatial Activity Pattern

The target spatial activity pattern may be based on activity of the subject or activity of other subjects or may be hypothetical.

When the target spatial activity pattern is based on other people, it may be from subjects who have achieved a desired mental, cognitive, emotional, or behavioral state or process. Similarly, when the target spatial activity pattern is hypothetical, it may be based on a target spatial activity pattern that is hypothesized to be desirable for a given mental, cognitive, emotional, or behavioral state or process.

The target spatial activity pattern may also be based on a measurement taken after the administration of a pharmaceutical agent that produces a desired outcome. Accordingly, the testing can be designed to test a subject in achieving the results that a pharmaceutical agent provides, or in testing dosage.

The target spatial activity pattern can also be measured for a subject when the subject reports a positive mental state or experience.

The target spatial activity pattern can also be measured for a subject when the subject performs positively in some task.

The target spatial activity pattern can also be measured for a subject by measuring the average spatial activity pattern during some class of events, such as during trial periods when the subject performed appropriately on a behavioral trial, or by comparing the spatial pattern of activity during trial periods when the subject performed appropriately on a behavioral trial with trial periods when the subject did not perform appropriately, or based upon the average event-related activity at a particular point during an activity.

The target spatial activity pattern can also be defined by measuring the average pattern of activity in a group of subjects. For example, if a set of subjects that have a particular condition, such as depression, show an average spatial activity pattern that is different from normal subjects, then this spatial activity pattern, or its opposite in this case, can be used as a testing target. In the case of depression, it has been shown that normal subjects on average have a higher pattern of activation in particular geometrically defined regions of the prefrontal cortex than do depressed subjects. This pattern can be measured as a spatial activity pattern that is the voxel-by-voxel difference between the activity in normal control subjects minus the activity in depressed subjects. The negative of this pattern may be used as a target state for testing.

B. Testing the Subject

Once a target state has been defined, a subject may be tested according to the present invention where the subject's brain activity in one or more regions of interest is monitored as the subject performs testing exercises. In this instance, the subject is communicated information regarding how the subject is performing relative to the target state. This may take place through the computation and display of an activity metric measuring the difference between the current activity state and a target state. The subject may be provided with the same or different stimuli/behaviors over time in effects to improve upon how the subject's current state compares with the target state.

C. Comparing the Target State to the Subject'S Current State

Provided herein is an example of how the target state may be compared to the subject's current state. It should be noted that other methods of comparison may also be devised and employed in conjunction with the invention.

In this example, an activity metric is defined that is the vector-difference of the currently observed spatial activity pattern within a region of interest and the target spatial activity pattern within the region of interest.

The subject may be tested to decrease this activity metric so that the activity metric increasingly approaches the desired target state. In this way, the intervention employed is intended to bring current state/process closer and closer to the target state/process.

If the target state involves regions to increase and regions to decrease, then the activity metric used in testing may be defined as:

(activity in each voxel to increase−background level)×voxel weight

(background level−activity in each voxel to decrease)×voxel weight

D. Communicating the Comparison to the Subject or Device Operator

The activity pattern information provided to a subject or device operator to allow the subject or device operator to have information for diagnosis or testing, or to match a desired target state can take a variety of forms.

For example, the information can be communicated quantitatively, as in the case of providing a visual or auditory readout of a number or graph corresponding to the defined activity metric, such as the vector difference between the target state and the current state.

The information can also be communicated qualitatively, as in the form of a tone that is of high frequency as the subject moves toward the target state/process, and low frequency as the subject moves away, or a digitized verbal indication. Visual objects can also be used to indicate this distance, such as graphical representations that indicate distance between two points, or the size or color of a visual indicator.

21. Selecting Tasks and Testing to Appropriate Level of Challenge and Dosing

The present invention may also be used to set appropriate levels of challenge for interventions that are to be undertaken by subjects either inside or outside of the measurement of physiological information, based upon the patterns of physiological activation that are evoked by those interventions during measurement. This may include dosing of pharmacological agents, or adjustment of stimulation parameters for nervous system stimulation devices. When a subjects activation does not reach a desired criterion, spatial activity patterns are measurably different than in the condition when the subject does reach the criterion. Therefore, this method includes measuring the average pattern of activity for more than one level of intervention, optionally determining a threshold level of intervention that leads to a defined level of activity, and then selecting interventions for the subject corresponding to a particular measured level of activity, such as a level above, at, or below the determined threshold. For each level of intervention, the average pattern of activity may be determined. A threshold may then be selected as a level of intervention that leads to a particular level of activity, or a particular percent of trials where an activity metric reaches a criterion level. With this information, it is possible to adjust intervention level or rate to be at or near the threshold of the subject's ability to achieve a given physiological response and to correctly perform the task. This process may be used in selecting pharmacological dosing or regimen, or in selecting parameters for other therapeutic interventions.

A subject or device operator may also use the trial-by-trial information about the spatial activity pattern measured to develop strategies for improving the efficacy of the intervention. As some spatial activity patterns are associated with positive outcomes, such as high activation or symptom amelioration, and others are associated with negative outcomes, such as negative side effects, subjects or device operators may adjust their intervention on each trial and their strategy overall to produce more beneficial outcomes.

22. Behavior, Movement, Rehabilitative, Performance and Sports Testing

Sports and performance testing may be facilitated using the methods of the present invention. It is known that practice, as well as mental rehearsal in the absence of actual activity, can improve performance in a variety of tasks and activities. Testing according to the present invention may be used to guide the practice or mental rehearsal of an activity in order to produce faster and more effective learning than practice or mental rehearsal would achieve without such assistance.

For example, the behavior employed in testing may be a mental rehearsal, such as a musician rehearsing a piece of music. In such case, the musician might be shown music and mentally envision himself conducting. Meanwhile, the musician's brain activity in regions of the brain associated with either reading music or imaging conducting could be measured. Using this information, the musician can learn to achieve a higher level of performance when practicing. Achieving a higher level of brain activity will enhance the effectiveness of such practice.

As can be seen, testing a subject in this manner teaches the subject how to more closely reproduce the target pattern of activity, either during the performance of the activity, or during mental rehearsal of the activity.

This type of mental testing may have has a variety of different uses. Take for example subjects who have lost or impaired control of movement due to congenital abnormalities, injuries, or cognitive or psychological impairments. With these subjects, it may be possible to determine which types of states or processes lead to the best performance of certain behaviors, and coach the subjects to increasingly produce those types of states or processes based upon the observed activity patterns.

23. Testing Methodologies

This invention has provided for means of testing subjects in the modulation of particular brain regions. This testing may take place using a variety of testing methodologies. In one example, the testing of subjects to control physiological activity takes place using classical conditioning. In another example, the testing of subjects to control physiological activity takes place using operant conditioning methods. In another example, the testing of subjects to control physiological activity takes place using psychophysical methods measuring a physiological measure such as an activity metric from a region of interest rather than a behavioral performance measure.

24. Defining Optimal Stimuli or Instructions for Behavior Using Reverse Correlation

This example illustrates one method for defining the optimal stimulus/behavior for a region of interest by using reverse correlation. This method may be used to define a linear estimate of the optimal stimulus to activate a given region of interest.

According to this example, a large number of stimuli may be presented. An average stimulus may be computed before periods when a measured activity level metric reaches a defined threshold. The stimuli typically contain many parts, such as a checkerboard visual stimulus with each square independently turning on and off, or an auditory stimulus with many tonal components. In this example, the average stimulus may then be computed by taking the average of each checkerboard square or auditory stimulus whenever the activity in a particular voxel reaches a threshold of two standard deviations above its own mean. Reverse correlation may also be performed using movements, rather than stimuli, as the input in order to compute the average movement before a measured activity metric. Reverse correlation methods have been described using many other types of physiological recording, such as single neuron recording, and one skilled in the art will be aware of how to apply this method in the context of the present invention to estimate stimuli to generate brain activation.

25. Single Point Measurement Device

In addition to using a scanning a fMRI instrument, a function magnetic resonance signal can be measured using a device that measures physiological activation levels from a single discretely localized fixed point or small volume. This measurement device may be a device that makes functional magnetic resonance measurements from a single location. Measurements from a single measurement point may be used in the testing of a subject as provided for in the remainder of this invention. Measurements from a single measurement point may be used in selection and triggering of measured information, stimuli, and instructions for other uses as provided for in this invention. In this case, the single measurement point may be used as the region of interest. This has special advantages with regard to the present invention as the present invention may be successfully used with an apparatus that makes measurements from a discretely localized region deep within the brain, even in the absence of the ability to scan the entire brain. A single point measurement device focuses data collection on a single point, the region of interest, rather than spreading measurement capacity over a larger brain volume. This focusing of acquisition leads to a proportionately larger number of sample measurement points that can be collected from the region of interest, as well as proportionately faster processing of the data. A single point measurement device may be used for this invention by the use of a scanning apparatus adjusted to collect data from only a single voxel, or a small group of voxels. A typical contemporary MRI scanner such as a GE 3.0 T Signa MRI scanner may be used as an embodiment of a single-point measurement device for magnetic resonance measurements. In order to make measurements in this mode, the scanning software must be configured to make repeated scans from a single voxel at high scan rate, or from a small number of small voxels that are then in turn averaged to effectively yield a single volume MR measurement. Thereby, a correspondingly increased sampling rate is possible.

In addition, a single point measurement apparatus may be used that does not include the ability to scan its measurement point in three dimensions, or that does not include the ability to scan its measurement point at all. A device of this type may be considerably simpler, and requires less expense than typical MRI scanning devices. For example, the device may have a single or small number of radiofrequency (RF) transmitters and receivers that are used to load RF energy into biological tissue and then measure the radiation which emerges. Rather than constructing a full tomographic image, a single point measurement device uses one or a number of selected locations for continuous measurement. This obviates the need for large and expensive tomographic instrumentation and computer reconstruction. The feature of this example is the ability to measure an fMRI signal from a particular point within the body without full tomographic reconstruction. In addition, the requirements for the magnetic field are lessened, particularly the requirements for magnetic field homogeneity. In total, this makes it possible to make fMRI-based measurements from discrete locations within the body at a much lower cost than using conventional instruments.

A single point measurement device may be used in the context of the present invention as the means for measuring the activity level in a discretely localized region of the brain. It may also be used in the context of the present invention as the means for measuring the activity level in a discretely localized region of the brain used in testing. The device provides sequential measurements from the discretely localized region at rapid intervals in turn can be used for physiological measurement and testing.

In order to use a single point measurement device for measurement, testing, and exercise, the measurement point of the device must be accurately positioned with respect to the target region of interest for measurement. This can be achieved by using a stereotaxic methodology whereby the head of a subject is held in place using a holding means that will position the head precisely with respect to the MR measurement instrument. Stereotaxic placement of the head into an apparatus is well appreciated by one skilled in the art. The head can then be positioned into the desired location relative to the MR measurement instrument using manipulators for the stereotaxic equipment.

Prior to use of a single point measurement device, it may be desirable to localize a region of interest within a subject, and then to make measurements from this region of interest using the single point measurement device. The location of the region of interest for use can be pre-determined using an embodiment of this invention that allows full, scanned imaging, and thereby allows the localization of the region of interest using anatomical or physiological means as provided for by this application and described in sections 3 and 4. For example, the region of interest that will be used for single point measurement may be located by using the position of a known point or anatomical structure within the head of the subject. This can be accomplished using stereotaxic coordinates, and/or using coordinates defined in a standard coordinate space such as that described by the Talairach brain or MNI brain and described in neuroanatomical texts. Once this region of interest has been located, the single point measurement device can be localized with respect to the subject such that the point of measurement of the device corresponds with the point of the defined region of interest, such as by stereotaxic placement as described.

A single point measurement device may also be used in order to achieve an anatomical scanning of the internal tissue of the subject. This may be useful in localizing the region of interest for physiological measurement as provided for in this invention. Anatomical localization can be achieved by moving the relative positions of the subject's head with respect to the measurement device using a mechanical positioning means while taking successive measurements at each relative position. The positions and measurement values may be put together to form a two or three dimensional anatomical image of the internal structures of the subject, where each 2-D or 3-D position has a value corresponding to the measurement made from that position. In this way, it is possible to reconstruct the internal anatomical landmarks from within the subject by taking sequential measurements and generating an image based upon the positions and values of those measurements. These internal anatomical landmarks can be used to position the measurement device. In particular, the device can be positioned so that it is at the physical location corresponding to the portion of the anatomical scan just described that is desired as the region of interest for physiological measurement. It is also possible to scan the internal tissue of the subject be altering the magnetic field of the single point measurement device, which will change the position of the fixed point relative to the magnet, or by changing the center frequency, pulse sequence or other properties of the RF energy that is used for measurement, which may select a different point in the magnetic field for measurement. In the same way as with physical motion of the scan point, measurements may be taken from successive locations, and used to reconstruct a 2-D or 3-D image of the internal structures of the subject. This, in turn, may be used to select the appropriate magnetic field and RF energy for use in physiological measurements from the region of interest.

26. Multiple Subject Measurement Apparatus

An embodiment of the invention described herein uses a single scanning apparatus to scan two or more subjects at substantially the same time. One embodiment uses RF coils large enough to include the head of more than one subject. Another embodiment uses one set of RF coils for each subject being scanned. Another embodiment uses one RF transmitter, and one RF receiver for each subject being scanned.

27. Use in Combination with Other Interventions

The methods described in this invention may be used in combination with a number of different additional methods, as described here. This may be used to test, monitor or improve the effects of such methods.

A. Use in Combination with Pharmacology

It is recognized that the various methods according to the present invention may be performed in combination with pharmacological intervention. This may be used to test, monitor or improve the effects of pharmacological agents.

i. Monitoring Brain Activation Produced by Pharmacological Agents

Pharmacological treatments may also serve to produce activation patterns that are then measured using this invention. For example, a given pharmacological agent may be administered to a subject. The subject's physiological states or processes may then be measured in the presence of the pharmacological agent that creates a state of activation or activity metric within the patient. These measurements can then be used to define an activation pattern for the patient for use in determining a region of interest, as provided for in section 4, and a pattern of activation for measurement, as provided for in Examples section 1.

Testing may be used to monitor the activity provided by a pharmacological agent. According to this variation, brain activity in selected regions is measured with and without the pharmacological agent, or during different time points in treatment, and regions of interest are defined as regions with a selective difference in activation between these two conditions. Then, those identified regions of interest are targeted to be tested according to the present invention.

In the example case of Parkinson's disease, any pharmacological agent that ameliorates Parkinson's disease symptoms may be used. Particular examples include, but are not limited to: L-dopa, pergolide, bromocryptine, promipexole and ropinirole. When a patient has been administered one of these agents and shows improved symptoms, brain activity may be measured in all or part of the brain. This activity may be compared with activity in the absence of the agents, or when symptoms are worsened. The activity pattern measured during successful treatment with one of these agents, or the difference between the pattern measured during successful treatment and without successful treatment, may be used as an activity pattern for testing.

As another example, prozac (fluoxetine) leads to an increase in activation of certain frontal areas of the patient. It may be possible to test subjects for increase the activation of those areas through neural activity exercises, either in the presence or absence of prozac (fluoxetine). These methods may be used in the development of pharmacological agents, such as by screening for agents which produce particular activity patterns. It should be noted that a pattern defined in one patient or group of patient, after administration of a pharmacological agent, can be used as a basis of comparison with a later patient or group of patients. In one instance, a patient may be administered more than one pharmacological agent at successive, separated times, with the brain activity patterns measured separately for each agent. Then, the patient may be administered the agent producing a measured pattern most similar to a desired target activity pattern, such as one shown to correspond with positive outcomes in a previous patient cohort.

This methodology may be employed in selecting an appropriate pharmacological dosage to achieve a desired measured activation level. This methodology may also be employed in selecting an appropriate pharmacological regimen to achieve a desired measured activation level. This methodology may also be employed in patient staging, and in monitoring the effects of a pharmacological or other treatment regimen over time.

ii. Reducing the Side-Effects of Pharmacological Agents

In another example, this invention may be used to reduce or alleviate side-effects produced by pharmacological intervention. Subjects taking a given drug may experience side effects, and these side effects may be correlated with an observable brain activity pattern in a particular region of interest, or in the whole brain. In order to reduce the presence of side effects of the drug, the subject may be tested to determine agents producing less of the undesired activity pattern associated with the unwanted side effect. As an example, certain dopaminergic antagonist drugs used to treat schizophrenia can produce undesirable side-effects reminiscent of Parkinson's disease, including paucity of motion, tremors, and other motor disturbances. These side effects are thought to arise through the inactivation of dopaminergic projections that are somewhat analogous to the inactivation pattern observed in Parkinson's patients. The drugs themselves produce altered patterns of activity within the brains of subjects taking the drugs. Therefore, these unwanted side effects can be avoided by selecting medication and dosing that avoid activity patterns associated with unwanted side-effects.

B. Use in Combination with Pharmacological Testing

It is envisioned that the present invention may also be used to determine the likely long-term success outcome of a pharmacological treatment, or to set appropriate dosage for that treatment.

It is noted in regard to this section that the subject used here may not be human but rather may be another mammal, such as a mouse, rat, rabbit, cat, dog, monkey, sheep, pig, or cow that is to be used in testing. Because such animals do not have the cognitive ability of humans to receive and process instructions, it is recognized that the stimuli or instructions for behavior used will necessarily be limited to those stimuli or instructions for behavior that the animal can be effectively asked to perform or which the animal can be made to perform. For example, the stimulus may be an external stimulus such as a sound, a smell, a bright light, or a nociceptive stimulus, that is applied to the animal. Another alternative is that measurements be made either at rest, or during sleep or sedation.

According to one embodiment, a subject's brain activation pattern is measured in a rest state, and may be repeatedly measured during the performance of testing. The subject is then administered a drug that is to be tested. After which, the subject repeats the rest state and the performance of the task in the presence of the drug. By comparing the resulting activity patterns (e.g., rest with the drug to rest without the drug; activity from testing with and without the drug; with and without the drug; the difference between rest and activity from testing with the drug as compared to the difference between rest and activity from testing without the drug), valuable information may be garnered regarding the activity pattern caused by the drug, the effect the drug has on task-activation or resting-state correlation measures, as well as brain drug metabolism.

These types of measures of brain activity may be used to indicate whether a pharmacological treatment is likely to lead to successful treatment outcomes in a given subject, or in a population. For example, the measured pattern of activity found with one or more drugs that were successful may be noted, as well as the measured pattern associated with one or more drugs that were unsuccessful. These measures may be made by taking the average pattern of activity for a successful drug or an unsuccessful drug across a population of subjects. In order to perform this averaging, standard methods may be used so that the activity pattern for each subject is appropriately normalized and geometrically transformed into a standard coordinate space to allow averaging.

A likelihood of positive outcome measure may then be determined for a given drug based upon the similarity of the activity pattern that it evokes with the pattern previously established to be associated with successful treatment. This pattern may correspond to a spatial pattern over many voxels, to an average activity level within a particular area or another selected region or combination of regions of the brain.

For pharmaceutical development, the measure of likelihood of positive outcome may be used as a ‘surrogate endpoint’ for successful treatment, and can be used to screen potential pharmaceutical candidates. This can take place either in humans, or in non-human animals used in pharmaceutical testing. In the case of selecting the most effective drug for a particular subject, a series of drugs may be sequentially tested in the same subject in this way, with the drug selected being the one that leads to the activity pattern most similar to the pattern observed for successful treatment in previous subjects in the past.

A similar process can also be used to detect drugs that are likely to lead to negative consequences or unwanted side-effects. In this case, rather than comparing the activity pattern measured during testing, behavior or rest in association with a positive outcome, the comparison may be made with the activity pattern measured during testing, behavior or rest in association with a negative outcome or undesired side-effect. Drugs that lead to similar activity patterns to those with negative outcomes may, of course, be avoided.

This method may also be used in order to determine appropriate pharmaceutical dosing, either for a new drug for which an appropriate dosage has not been set, or for an existing drug for which a dosage needs to be set for a particular individual. In either case, the dosage of the drug can be set as the minimum dose required to evoke a given level of the activity pattern associated with a positive outcome, such as successful treatment.

In the case of pharmaceutical development, the measure of likelihood of positive outcome is used as a surrogate endpoint for successful treatment, and can be used to screen potential pharmaceutical candidates. This can take place either in humans, or in non-human animals used in pharmaceutical testing. In the case of selecting a drug for a particular subject, a series of drugs can be tested in the same subject in this way, with the drug selected being the one that leads to the pattern most similar to the pattern observed for successful treatment in the past.

C. Combination with Stimulation Methods

It is envisioned that the present invention may also be used to determine the likely long-term success outcome of a nervous system stimulation treatment, or to set appropriate stimulation parameters for that treatment. Nervous system stimulation may include deep brain stimulation (DBS), trans-cutaneous magnetic stimulation (TMS), or other stimulation modalities.

It is noted in regard to this section that the subject used here may not be human but rather may be another mammal, such as a mouse, rat, rabbit, cat, dog, monkey, sheep, pig, or cow that is to be used in testing. Because such animals do not have the cognitive ability of humans to receive and process instructions, it is recognized that the stimuli or instructions for behavior used will necessarily be limited to those stimuli or instructions for behavior that the animal can be effectively asked to perform or which the animal can be made to perform. For example, the stimulus may be an external stimulus such as a sound, a smell, a bright light, or a nociceptive stimulus, that is applied to the animal. Another alternative is that measurements be made either at rest, or during sleep or sedation.

According to one embodiment, a subject's brain activation pattern is measured in a rest state, and may be repeatedly measured during the performance of testing. The stimulation paradigm being tested as an intervention may be applied during the course of testing, or at times intervening between subsequent tests.

Measures of brain activity may be used to indicate whether a stimulation intervention is likely to lead to successful treatment outcomes in a given subject, or in a population. For example, the measured pattern of activity found with one or more interventions that were successful may be noted, as well as the measured pattern associated with one or more interventions that were unsuccessful. These measures may be made by taking the average pattern of activity for a successful intervention or an unsuccessful intervention across a population of subjects. In order to perform this averaging, standard methods may be used so that the activity pattern for each subject is appropriately normalized and geometrically transformed into a standard coordinate space to allow averaging.

This method may also be used in order to determine appropriate intervention parameters, either for a new intervention for which an appropriate parameters have not been set, or for an existing intervention for which parameters need to be set for a particular individual. In either case, the parameters can be set as the minimum required to evoke a given level of the activity pattern associated with a positive outcome, such as successful treatment. Parameters may include stimulus intensity, duration, frequency, or repetition rate, as well as placement within or adjacent to the nervous system.

D. Combination with Additional Therapies and Methods

The present invention can be used in combination with a variety of additional and non-traditional therapies and methods including: rehabilitative massage, sports or other massage, guided visualization, meditation, biofeedback, hypnosis, relaxation techniques, acupressure, acupuncture. In each case, the subject can undergo the non-traditional therapy technique while undergoing testing. The non-traditional therapy technique can be used to enhance the subjects ability to succeed at testing to control and exercise a given brain region. In addition, the testing methodology can allow for improved outcomes based upon the use of these non-traditional therapeutic techniques.

i. Combination with Physical Therapy

The present invention can be performed in combination with physical therapy. In such case, the exercises that the subject undergoes during testing may exercises prescribed for physical therapy. The invention may be used to speed the improvement produced by the exercises of physical therapy. The invention may also be used to measure the improvement or change in brain functioning produced by physical therapy over the course of treatment. In addition, the subject can undergo physical therapy exercises as an adjunct to the use of this method.

ii. Combination with Psychological Counseling or Psychotherapy

This invention can be combined with psychological counseling or psychotherapy. The subject can undergo interchange with a psychological counselor or psychotherapist while undergoing measurement and testing as described in this invention to evaluate the person's response. For example, the therapy may relate to stress or anger management where how effectively stress or anger is being managed is measured during therapy. The subject can also undergo psychological counseling or psychotherapy as an adjunct to the use of this method.

28. Localization of Neuronal Function, Especially for Neurosurgery

The present invention may also be used to localize within the brain the correlates of certain psychological or neurological functions. For example, through testing it may be possible to determine the areas that are most activated by particular psychological or neurologic functions. If the physiological criteria selected are activation in correlation with a particular task, then the brain regions engaged during testing and performance of this task are determined. This can be used as a method for determining where areas are located. This may be useful in neurosurgery, such as for the sparing of regions or hemisphere involved in language (e.g. as a replacement for the traditional wada test), and regions involved in motor control.

29. Localization of Seizure Foci

The present invention may also be used to localize epileptic seizure foci by determining a pattern of activation during a seizure or preceding a seizure in comparison with the pattern of activation when a seizure is not taking place. This may be useful in preparing for neurosurgical ablation of a seizure focus, or in using testing to control seizures.

This technique may also be used to measure a degree of activation of different regions during a seizure, and the impact of particular medications on the activations of these areas during a seizure. This may be used to determine which medications are most likely to prevent or ameliorate seizure activity. This is made possible because the area of a seizure focus will typically show increased neurophysiological activation during a seizure, and hence is localized using these techniques and apparatus. The time course of a seizure may also be accurately mapped in three dimensions and in time.

30. Diagnosis and Treatment of Neurologic Injury or Disease

Methods are also provided for diagnosing and treating an area of the brain that has been compromised by a stroke or other cerebrovascular or other neurologic injury or neurological disease. According to these methods, the diagnosis and treatments may be conducted in combination with performing testing exercises and monitoring brain activity in regions of interest according to the present invention.

A. Mapping and Diagnosis of Areas of Injury or Disease

When a subject has had a neurologic injury, such as a stroke or other cerebrovascular or other neurologic injury, mapping is performed to determine what regions of the brain have been compromised by the injury. The extent or progression of the damage may also be evaluated. For example, anatomical mapping can provide one indication of the areas compromised by a cerebrovascular accident. A second indication of the areas of damage or partial disfunction may also be provided by performing physiological measurements of brain activity. In order to achieve this, the physiological activation patterns in subjects are measured, such as by measurements according to the present invention.

Mapping may be used as a diagnostic tool to detect areas that have been injuring. The diagnostic method may simply include measuring an activation pattern of a subject while the subject is presented with one or more stimuli and/or engaged in one or more behaviors that are designed to activate regions of interest of the brain thought to be potentially compromised by the neurologic injury. The activation may then be compared with activation when the subject is in a rest state in order to determine a background level of activity. The activation may also be compared with the activation observed in an unimpaired subject performing a comparable task.

Regions where no activation is observed can be surmised to be compromised zones. Regions where only low levels of activation or other abnormal activity metrics are observed in comparison with normal subjects undergoing the same tasks may be surmised to be partially compromised.

The variance measured in the activity level or other activity metric during a rest or task condition for any brain voxel can be used as an indicator of the state of the corresponding neural tissue. Voxels with very little of the normally observed fluctuation in the background level of activity can be surmised to be affected or compromised by neurologic injury. This may allow an automatic mapping process to take place for the regions affected by a given disease or condition.

31. Characterization of Brain Regions

An additional example of this invention relates to the characterization of brain regions of unknown or only partially known function. Through the use of this invention, it is possible to characterize the functioning of a localized brain region of interest. In this example, a brain region to be characterized is selected as a region of interest. A procedure is laid out for the testing of brain regions of interest in sections 1-6. Sections 4 and 5 describe the process of determining appropriate stimuli or behaviors to activate a brain region of interest. Thereby, this invention provides for a method for determining appropriate stimuli or behaviors to activate a brain region of interest in instances where the function of this region is incompletely understood. Once these stimuli or behaviors have been determined, this serves as a characterization of the function of this brain region of interest. It is possible to perform this characterization to generate new knowledge of the functions of a brain region. This knowledge of the characterization of a brain region may be used for a variety of purposes. For example, this new knowledge may be used to design treatments involving the characterized brain region of interest. These treatments may include pharmacological treatments, surgical treatments, electrical stimulation treatments, or other treatments. The knowledge of the characterization of a brain region may be used for diagnostic purposes as well. For instance, if it has been determined that a brain region of interest is implicated in a condition, such as a disease, then using the stimuli or behaviors determined to engage that brain region may be used as a diagnostic for whether a subject has that condition, and the extent or severity of the condition. These stimuli or behaviors determined to engage the brain region may also be used in conjunction with a pharmacological treatment as a means for determining the effect of the pharmacological treatment on the activation observed in the brain region of interest in the presence and absence of the pharmacological treatment. This may be used as a means for assessing the pharmacological treatment.

It will be apparent to those skilled in the art that various modifications and variations can be made to the methods, software and systems of the present invention. The foregoing examples and figures are presented for purposes of illustration and description. It is not intended to be exhaustive or to limit the invention to the precise forms disclosed. Many modifications and variations will be apparent to practitioners skilled in this art and are intended to fall within the scope of the invention.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

What is claimed is:

1. A computer assisted method is provided for diagnosing a condition of a subject wherein said condition is associated with an activation in one or more regions of interest, said method comprising:
having said subject perform a behavior or have a perception adapted to selectively activate said one or more regions of interest associated with said condition;
measuring activity of said one or more regions of interest as said behavior is being performed or said subject has the perception;
diagnosing said condition associated with said one or more regions of interest based on said activity in response to the behavior or perception;
performing an intervention; and
repeating this process one or more times at a later time and observing changes between measurements before and after said intervention.

2. A method according to claim 1 wherein said measuring activity is preformed by fMRI.

3. A method according to claim 1 wherein the measuring activity is made in less than 10 seconds relative to when the activity is measured.

4. A method according to claim 1 wherein said intervention comprises an application of a pharmacological agent.

5. A method according to claim 1 wherein said intervention comprises an application of a therapeutic method.

6. A method according to claim 1 wherein said diagnosing is made while an instrument used for measurement remains positioned about said subject.

7. A method according to claim 1 wherein said method further comprises selecting one or more of internal voxels corresponding to a region of interest for said subject and using said selected internal voxels to make one or more diagnoses.

8. A method according to claim 7 wherein said measuring is made using an apparatus capable of taking measurements from one or more of said internal voxels without substantial contamination of said measurements by activity from regions intervening between said internal voxels and location where said measurement apparatus collects the data.

9. A method according to claim 7 wherein said measuring is made from at least 100 separate internal voxels at a rate of at least once every five seconds.

10. A method according to claim 7 wherein said measuring is made from a set of separate internal voxels corresponding to a scan volume including the entire brain.

11. A method according to claim 7 wherein said measuring is made from at least 100 separate internal voxels and wherein said internal voxels have a total three-dimensional volume of 5×5×5 cm or less.

12. A method according to claim 7 wherein said measuring is made from at least 100 separate internal voxels and wherein said internal voxels have a total three-dimensional volume of 1×1×1 cm or less.

13. A method according to claim 7 wherein the region of interest is selected from the group consisting of subthalamic nucleus, substantia nigra, thalamic nucleus VA ventro anterior, nucleus accumbens, thalamic nucleus VL ventrolateral, globus pallidus internus, pulvinar nucleus, thalamic nucleus VP, locus coeruleus, globus pallidus externus, amygdala, medial frontal lobe, periaqueductal gray matter, nucleus raphe dorsalis, nucleus basalis of Meynert, dorsolateral pre-frontal cortex, anterior pre-frontal cortex, rostral ventromedial medulla, nucleus raphe magnus, thalamic nucleus Vim ventrointernomedial, Brodmann's area 4, Brodmann's area 6.

14. A method according to claim 1 wherein said one of said regions of interest has a primary function of releasing a neuromodulatory substance.

15. The method according to claim 14 wherein said neuromodulatory substance is selected from the group consisting of: dopamine, acetyl choline, noradrenaline, serotonin, and endogenous opiate.

16. A method according to claim 1 wherein said subject has one or more of the following conditions: Parkinson's disease, Alzheimer's disease, attention deficit disorder, depression, substance abuse, addiction, and schizophrenia.

17. A method according to claim 1 wherein information is communicated to said subject to perform a behavior or have a perception by a manner selected from the group consisting of: providing audio to the subject, providing tactile stimuli to the subject, providing a smell to the subject, displaying an image to the subject, communicating a set of instruction, and communicating material to be learned.

18. Computer executable software is provided for guiding brain activity testing, the software comprising: logic for communicating instructions to a subject to perform a first behavior and/or a first stimulus to the subject; logic for taking activity measurements of one or more regions of interest of the subject in response to the first behavior or first stimulus and selecting a second behavior or a second stimulus for activating the one or more regions of interest based, at least in part, on the measured brain activity; and logic for communicating instructions to the subject to perform the second behavior and/or the second stimulus to the subject; logic for testing the activity measurements and performing a diagnosis of the subject or of the efficacy of an applied intervention.

19. Software according to claim 18 wherein the software performs the determinations in less than 10 seconds relative to when the brain activity measurement is taken.

20. A method of diagnosing a subject comprising:
(a) measuring activity of one or more internal voxels of said subject's brain;
(b) communicating instructions to said subject derived from said measured activity in real time; and
(c) having said subject perform a behavior in response to said instructions in real time.

21. A method according to claim 20 wherein said measuring is performed by fMRI.

22. A method according to claim 20 wherein said measuring is made from at least 100 separate voxels.

23. A method according to claim 20 wherein said instructions are derived through a computer executable logic process of selecting from a set of possible instructions based upon the brain activity measured.

24. A method according to claim 20 wherein a computer executable logic is employed to cause the instructions to be communicated to the subject.

25. Computer executable software, the software comprising:
logic for taking activity measurements of one or more localized brain regions as an intervention is performed and for communicating information to a subject or a device operator based on measured brain activity in substantially real time relative to when the intervention is performed,
wherein the logic takes new activity measurements as they are received and communicates new information based on new activity measurements.
